(19) United States
(12) Patent Application Publication
(10) Pub. No.: US 2012/0277158 A1
(43) Pub. Date: Nov. 1, 2012

(54) COMPOSITIONS AND METHODS FOR THE TRANSPORT OF THERAPEUTIC AGENTS

(75) Inventors: Jean-Paul Castaigne, Mont-Royal (CA); Michel Demeule, Beaconsfield (CA); Christian Che, Longueuil (CA); Anthony Regina, Montreal (CA)

(73) Assignee: Angiochem Inc., Montreal, QC (CA)

(21) Appl. No.: 13/500,852

(22) PCT Filed: Oct. 5, 2010
(86) PCT No.: PCT/CA2010/001596

§ 371 Date: Jul. 9, 2012

Related U.S. Application Data
(60) Provisional application No. 61/249,152, filed on Oct. 6, 2009.

(51) Int. Cl. A61K 38/10; C07K 9/00; C07K 14/00; C07K 14/475; C07K 14/575; A61K 38/16; A61P 25/28; A61P 25/18; A61P 25/00; A61P 25/08; A61P 25/16; A61P 43/00; A61P 3/00; A61P 35/00; A61P 35/04; C07K 1/113; C07K 7/08; B82Y 5/00

(52) U.S. Cl. 514/17.5; 530/326; 530/322; 530/325; 530/324; 530/409; 530/399; 514/21.4; 514/21.3; 514/21.5; 514/17.7; 514/17.9; 514/17.8; 514/19.3; 514/19.8; 977/788; 977/906

(57) ABSTRACT

The present invention is directed to conjugates that include a polypeptide capable of crossing the blood-brain barrier or entering one or more cell types attached to a transport vector, i.e., a composition capable of transporting an agent (e.g., a therapeutic agent). In certain cases, the polypeptides are directly conjugated to a lipid or polymeric vector to allow targeted application of a therapeutic agent to treat, for example, a cancer, a neurodegenerative disease, or a lysosomal storage disorder.

### FIELD OF THE INVENTION

The present invention relates to the polypeptide-transport vector conjugates and use of the conjugates for transporting agents (e.g., therapeutic agents) across the blood-brain barrier or into other cells, tissues, or organs of a subject (e.g., for the treatment of diseases such as cancer, neurodegenerative diseases, and lysosomal storage diseases).

### BACKGROUND OF THE INVENTION

In the development of a new therapy for brain pathologies, the blood-brain barrier (BBB) is considered a major obstacle for the potential use of drugs for treating disorders of the central nervous system (CNS). The global market for CNS drugs was $33 billion in 1998, which was roughly half that of global market for cardiovascular drugs, even though in the United States, nearly twice as many people suffer from CNS disorders as from cardiovascular diseases. The reason for this imbalance is, in part, that more than 98% of all potential CNS drugs do not cross the BBB. In addition, more than 99% of worldwide CNS drug development is devoted solely to CNS drug discovery, and less than 1% is directed to CNS drug delivery. This may explain the lack of therapeutic options available for major neurological diseases.

The brain is shielded against potentially toxic substances by the presence of two barrier systems: the BBB and the blood-cerebrospinal fluid barrier (BCSFB). The BBB is considered to be the major route for the uptake of serum ligands since its surface area is approximately 5000-fold greater than that of BCSFB. The brain endothelium, which constitutes the BBB, represents the major obstacle for the use of potential drugs against many disorders of the CNS. As a general rule, only small lipophilic molecules may pass across the BBB, i.e., from circulating systemic blood to brain. Many drugs that have a larger size or higher hydrophobicity show high efficacy in CNS targets but are not efficacious in animals as these drugs cannot effectively cross the BBB. Thus, peptide and protein therapeutics are generally excluded from transport from blood to brain, owing to the negligible permeability of the brain capillary endothelial wall to these drugs. Brain capillary endothelial cells (BCECs) are closely sealed by tight junctions, possess few fenestrae and few endocytic vesicles as compared to capillaries of other organs. BCECs are surrounded by extracellular matrix, astrocytes, pericytes, and microglial cells. The close association of endothelial cells with the astrocyte foot processes and the basement membrane of capillaries are important for the development and maintenance of the BBB properties that permit tight control of blood-brain exchange.

Thus, improved means for transporting therapeutic agents across the BBB is highly desirable.

### SUMMARY OF THE INVENTION

The present invention features polypeptide-transport vector conjugates that are capable of transporting a therapeutic agent across the blood-brain barrier (BBB) or into a cell. The transport vector may contain any therapeutic agent, including RNAi agents, polynucleotides (e.g., encoding RNAi agents), anticancer therapeutics, small molecule drugs, polypeptide therapeutics, and hydrophobic agents. The conjugates of the invention are especially useful in treatment of diseases where increased intracellular delivery or delivery across the BBB is desirable. The conjugates may be used to treat a cancer, a neurodegenerative disease, a lysosomal storage disease, or any disease or condition described herein. The invention also features methods of making polypeptide-transport vectors.

Accordingly, in one aspect, the invention features a polypeptide-transport vector conjugate. The conjugate may be a compound of the formula:

A-X-B

where A is a targeting polypeptide; X is a linker; and B is a transport vector.

In a second aspect, the invention features the invention features a method of treating a subject having disease such as a cancer (e.g., metastatic cancer), a neurodegenerative disease, or a lysosomal storage disorder or any disease or disorder described herein, by administering a polypeptide-transport vector conjugate to the subject in a therapeutically effective amount. In certain embodiments, the disorder or disease is amenable to treatment with a GLP-1 agonist, leptin or a leptin analog, neurotensin or a neurotensin analog, glial-derived neurotrophic factor (GDNF) or an analog thereof, or brain-derived neurotrophic factor (BDNF) or an analog thereof. Many such diseases and disorders are described herein. The disease may be listed in Table 2 and the conjugate may be bound to or may contain a therapeutic agent capable of treating a disease listed in Table 2 (e.g., an RNAi agent directed against the targets listed in Table 2, a nucleic acid encoding the RNAi agent, or a nucleic acid expressing the indicated protein). In embodiments where the disease is cancer, the therapeutic agent is an anticancer agent. The cancer may be a brain or central nervous system (CNS) cancer, such as a brain tumor (e.g., a glioma or glioblastoma), brain tumor metastasis, or a tumor that has metastasized, or may be a hepatocellular carcinoma, lung cancer, or any of the cancers (e.g., metastatic cancer) described herein. In other embodiments, the conjugate contains a therapeutic capable of treating schizophrenia, epilepsy, stroke, or any neurodegenerative disease described herein. In other embodiments, the lysosomal storage disease is Wolman's disease or any lysosomal storage disorder described herein (e.g., as described in Table 2 herein).

In another aspect, the invention features a method of making a polypeptide-transport vector conjugate. The method includes conjugating a polypeptide to a transport vector, where the polypeptide is exposed on the outer surface of the vector. The method may further include a step of encapsulating a therapeutic agent in the vector or attaching a therapeutic onto the vector, either prior to or following the conjugation. In certain embodiments, the lipid vector includes a tether molecule on its outer surface, and the conjugating step includes conjugating the polypeptide to the tether molecule.

In a related aspect, the invention features a method of making a polypeptide-transport vector conjugate. The method includes conjugating a polypeptide to either a molecule capable of forming the transport vector (e.g., a lipid, a carbohydrate, or a biocompatible polymer) or a tether molecule conjugated to the molecule capable of forming the transport vector, thereby forming a conjugate, and forming a transport vector including the conjugate. The polypeptide can be exposed on the surface of the vector. The method may further include encapsulating a therapeutic agent in the vector.

In any of the above aspects, the targeting polypeptide may be substantially identical (e.g., having at least 50%, 60%, 70%, 75%, 80%, 85%, 90%, 95%, 96%, 97%, 98%, 99%, or 100% identity) to any of the sequences set forth in Table 1, or a functional fragment thereof (e.g., having truncations of one or more (e.g., 2, 3, 4, 5, 6, 7, 8, 9, 10, 11, 12, 13, 14, 15, 16, 17, 18, or 19) amino acids wherein the truncation may originate from the amino terminus (N-terminus), carboxy terminus (C-terminus), or from the interior of the protein). In certain embodiments, the polypeptide has a sequence of Angiopep-1 (SEQ ID NO:67), Angiopep-2 (SEQ ID NO:97), Angiopep-3 (SEQ ID NO:107), Angiopep-4-a (SEQ ID NO:108), Angiopep-4-b (SEQ ID NO:109), Angiopep-5 (SEQ ID NO:110), Angiopep-6 (SEQ ID NO:111), or Angiopep-7 (SEQ ID NO:112). The targeting polypeptide or polypeptide-transport vector conjugate may be efficiently transported into a particular cell type (e.g., any one, two, three, four, or five of liver, lung, kidney, spleen, and muscle) or may cross the mammalian BBB efficiently (e.g., Angiopep-1, -2, -3, -4-a, -4-b, -5, and -6). In another embodiment, the targeting polypeptide or polypeptide-transport vector conjugate is able to enter a particular cell type (e.g., any one, two, three, four, or five of liver, lung, kidney, spleen, and muscle) but does not cross the BBB efficiently (e.g., Angiopep-7). The targeting polypeptide may be of any length, for example, at least (or at most) 6, 7, 8, 9, 10, 11, 12, 13, 14, 15, 16, 17, 18, 19, 20, 21, 25, 35, 50, 75, 100, 200, or 500 amino acids. In certain embodiments, the targeting polypeptide is 10 to 50 amino acids in length. The conjugate may be substantially pure. The targeting polypeptide may be produced by recombinant genetic technology or chemical synthesis. The conjugate can be formulated with a pharmaceutically acceptable carrier.

TABLE 1
Exemplary Targeting Polypeptides

| SEQ | |
|---|---|
| ID | |
| NO: | |
| 1 | T F V Y G G C R A K R N N F K S A E D |
| 2 | T F Q Y G G C M G N G N N F V T E K E |
| 3 | P F F Y G G C G G N R N N F D T E E Y |
| 4 | S F Y Y G G C L G N K N N Y L R E E E |
| 5 | T F F Y G G C R A K R N N F K R A K Y |
| 6 | T F F Y G G C R G K R N N F K R A K Y |
| 7 | T F F Y G G C R A K K N N Y K R A K Y |
| 8 | T F F Y G G C R G K K N N F K R A K Y |
| 9 | T F Q Y G G C R A K R N N F K R A K Y |
| 10 | T F Q Y G G C R G K K N N F K R A K Y |
| 11 | T F F Y G G C L G K R N N F K R A K Y |
| 12 | T F F Y G G S L G K R N N F K R A K Y |
| 13 | P F F Y G G C G G K K N N F K R A K Y |
| 14 | T F F Y G G C R G K G N N Y K R A K Y |
| 15 | P F F Y G G C R G K R N N F L R A K Y |
| 16 | T F F Y G G C R G K R N N F K R E K Y |
| 17 | P F F Y G G C R A K K N N F K R A K E |
| 18 | T F F Y G G C R G K R N N F K R A K D |
| 19 | T F F Y G G C R A K R N N F D R A K Y |
| 20 | T F F Y G G C R G K K N N F K R A E Y |
| 21 | P F F Y G G C G A N R N N F K R A K Y |
| 22 | T F F Y G G C G G K K N N F K T A K Y |
| 23 | T F F Y G G C R G N R N N F L R A K Y |
| 24 | T F F Y G G C R G N R N N F K T A K Y |
| 25 | T F F Y G G S R G N R N N F K T A K Y |
| 26 | T F F Y G G C L G N G N N F K R A K Y |
| 27 | T F F Y G G C L G N R N N F L R A K Y |
| 28 | T F F Y G G C L G N R N N F K T A K Y |
| 29 | T F F Y G G C R G N G N N F K S A K Y |
| 30 | T F F Y G G C R G K K N N F D R E K Y |
| 31 | T F F Y G G C R G K R N N F L R E K E |
| 32 | T F F Y G G C R G K G N N F D R A K Y |
| 33 | T F F Y G G S R G K G N N F D R A K Y |
| 34 | T F F Y G G C R G N G N N F V T A K Y |
| 35 | P F F Y G G C G G K G N N Y V T A K Y |
| 36 | T F F Y G G C L G K G N N F L T A K Y |
| 37 | S F F Y G G C L G N K N N F L T A K Y |
| 38 | T F F Y G G C G G N K N N F V R E K Y |
| 39 | T F F Y G G C M G N K N N F V R E K Y |
| 40 | T F F Y G G S M G N K N N F V R E K Y |
| 41 | P F F Y G G C L G N R N N Y V R E K Y |
| 42 | T F F Y G G C L G N R N N F V R E K Y |
| 43 | T F F Y G G C L G N K N N Y V R E K Y |
| 44 | T F F Y G G C G G N G N N F L T A K Y |
| 45 | T F F Y G G C R G N R N N F L T A E Y |
| 46 | T F F Y G G C R G N G N N F K S A E Y |
| 47 | P F F Y G G C L G N K N N F K T A E Y |
| 48 | T F F Y G G C R G N R N N F K T E E Y |
| 49 | T F F Y G G C R G K R N N F K T E E D |
| 50 | P F F Y G G C G G N G N N F V R E K Y |
| 51 | S F F Y G G C M G N G N N F V R E K Y |
| 52 | P F F Y G G C G G N G N N F L R E K Y |
| 53 | T F F Y G G C L G N G N N F V R E K Y |
| 54 | S F F Y G G C L G N G N N Y L R E K Y |
| 55 | T F F Y G G S L G N G N N F V R E K Y |
| 56 | T F F Y G G C R G N G N N F V T A E Y |
| 57 | T F F Y G G C L G K G N N F V S A E Y |
| 58 | T F F Y G G C L G N R N N F D R A E Y |
| 59 | T F F Y G G C L G N R N N F L R E E Y |
| 60 | T F F Y G G C L G N K N N Y L R E E Y |
| 61 | P F F Y G G C G G N R N N Y L R E E Y |
| 62 | P F F Y G G S G G N R N N Y L R E E Y |
| 63 | M R P D F C L E P P Y T G P C V A R I |
| 64 | A R I I R Y F Y N A K A G L C Q T F V Y G |
| 65 | Y G G C R A K R N N Y K S A E D C M R T C G |
| 66 | P D F C L E P P Y T G P C V A R I I R Y F Y |
| 67 | T F F Y G G C R G K R N N F K T E E Y |
| 68 | K F F Y G G C R G K R N N F K T E E Y |
| 69 | T F Y Y G G C R G K R N N Y K T E E Y |
| 70 | T F F Y G G S R G K R N N F K T E E Y |
| 71 | C T F F Y G C C R G K R N N F K T E E Y |
| 72 | T F F Y G G C R G K R N N F K T E E Y C |
| 73 | C T F F Y G S C R G K R N N F K T E E Y |
| 74 | T F F Y G G S R G K R N N F K T E E Y C |
| 75 | P F F Y G G C R G K R N N F K T E E Y |
| 76 | T F F Y G G C R G K R N N F K T K E Y |
| 77 | T F F Y G G K R G K R N N F K T E E Y |
| 78 | T F F Y G G C R G K R N N F K T K R Y |
| 79 | T F F Y G G K R G K R N N F K T A E Y |
| 80 | T F F Y G G K R G K R N N F K T A G Y |
| 81 | T F F Y G G K R G K R N N F K R E K Y |
| 82 | T F F Y G G K R G K R N N F K R A K Y |
| 83 | T F F Y G G C L G N R N N F K T E E Y |
| 84 | T F F Y G C G R G K R N N F K T E E Y |
| 85 | T F F Y G G R C G K R N N F K T E E Y |
| 86 | T F F Y G G C L G N G N N F D T E E E |
| 87 | T F Q Y G G C R G K R N N F K T E E Y |
| 88 | Y N K E F G T F N T K G C E R G Y R F |
| 89 | R F K Y G G C L G N M N N F E T L E E |
| 90 | R F K Y G G C L G N K N N F L R L K Y |
| 91 | R F K Y G G C L G N K N N Y L R L K Y |
| 92 | K T K R K R K K Q R V K I A Y E E I F K N Y |
| 93 | K T K R K R K K Q R V K I A Y |
| 97 | T F F Y G G S R G K R N N F K T E E Y |
| 98 | M R P D F C L E P P Y T G P C V A R I |
| | I R Y F Y N A K A G L C Q T F V Y G G |
| | C R A K R N N F K S A E D C M R T C G G A |
| 99 | T F F Y G G C R G K R N N F K T K E Y |
| 100 | R F K Y G G C L G N K N N Y L R L K Y |
| 101 | T F F Y G G C R A K R N N F K R A K Y |
| 102 | N A K A G L C Q T F V Y G G C L A K R N N F |
| | E S A E D C M R T C G G A |
| 103 | Y G G C R A K R N N F K S A E D C M R T C G |
| | G A |
| 104 | G L C Q T F V Y G G C R A K R N N F K S A E |
| 105 | L C Q T F V Y G G C E A K R N N F K S A |
| 107 | T F F Y G G S R G K R N N F K T E E Y |
| 108 | R F F Y G G S R G K R N N F K T E E Y |
| 109 | R F F Y G G S R G K R N N F K T E E Y |
| 110 | R F F Y G G S R G K R N N F R T E E Y |
| 111 | T F F Y G G S R G K R N N F R T E E Y |
| 112 | T F F Y G G S R G R R N N F R T E E Y |
| 113 | C T F F Y G G S R G K R N N F K T E E Y |
| 114 | T F F Y G G S R G K R N N F K T E E Y C |
| 115 | C T F F Y G G S R G R R N N F R T E E Y |
| 116 | T F F Y G G S R G R R N N F R T E E Y C |

Polypeptides Nos. 5, 67, 76, and 91, include the sequences of SEQ ID NOS: 5, 67, 76, and 91, respectively, and are amidated at the C-terminus.
Polypeptides Nos. 107, 109, and 110 include the sequences of SEQ ID NOS: 97, 109, and 110, respectively, and are acetylated at the N-terminus.

In any of the above aspects, the targeting polypeptide may include an amino acid sequence having the formula:

X1-X2-X3-X4-X5-X6-X7-X8-X9-X10-X11-X12-X13-X14-X15-X16-X17-X18-X19

where each of X1-X19 (e.g., X1-X6, X8, X9, X11-X14, and X16-X19) is, independently, any amino acid (e.g., a naturally occurring amino acid such as Ala, Arg, Asn, Asp, Cys, Gln, Glu, Gly, His, Ile, Leu, Lys, Met, Phe, Pro, Ser, Thr, Trp, Tyr, and Val) or absent and at least one (e.g., 2 or 3) of X1, X10, and X15 is arginine. In some embodiments, X7 is Ser or Cys; or X10 and X15 each are independently Arg or Lys. In some embodiments, the residues from X1 through X19, inclusive, are substantially identical to any of the amino acid sequences of any one of SEQ ID NOS:1-93, 97-105 and 107-116 (e.g., Angiopep-1, Angiopep-2, Angiopep-3, Angiopep-4-a, Angiopep-4-b, Angiopep-5, Angiopep-6, and Angiopep-7). In some embodiments, at least one (e.g., 2, 3, 4, or 5) of the amino acids X1-X19 is Arg. In some embodiments, the polypeptide has one or more additional cysteine residues at the N-terminal of the polypeptide, the C-terminal of the polypeptide, or both.

In certain embodiments of any of the above aspects, the polypeptide is modified (e.g., as described herein). The polypeptide may be amidated, acetylated, or both. Such modifications to polypeptides may be at the amino or carboxy terminus of the polypeptide. The conjugates of the invention may also include peptidomimetics (e.g., those described herein) of any of the polypeptides described herein. The polypeptide may be in a multimeric form, for example, dimeric form (e.g., formed by disulfide bonding through cysteine residues).

In certain embodiments, the polypeptide has an amino acid sequence described herein with at least one amino acid substitution (e.g., 2, 3, 4, 5, 6, 7, 8, 9, 10, 11, or 12 substitutions). The polypeptide may contain, for example, 1 to 12, 1 to 10, 1 to 5, or 1 to 3 amino acid substitutions, for example, 1 to 10 (e.g., to 9, 8, 7, 6, 5, 4, 3, 2) amino acid substitutions. The amino acid substitution(s) may be conservative or non-conservative. For example, the polypeptide may gave an arginine at one, two, or three of the positions corresponding to positions 1, 10, and 15 of the amino acid sequence of any of SEQ ID NO:1, Angiopep-1, Angiopep-2, Angiopep-3, Angiopep-4-a, Angiopep-4-b, Angiopep-5, Angiopep-6, and Angiopep-7.

In any of the above aspects, the conjugate may specifically exclude a targeting polypeptide including or consisting of any of SEQ ID NOS:1-93, 97-105 and 107-116 (e.g., Angiopep-1, Angiopep-2, Angiopep-3, Angiopep-4-a, Angiopep-4-b, Angiopep-5, Angiopep-6, and Angiopep-7). In some embodiments, the polypeptides and conjugates of the invention exclude the polypeptides of SEQ ID NOs:102, 103, 104, and 105.

In any of the above aspects, the targeting polypeptide may be conjugated to the transport vector directly (e.g., through hydrophobic, covalent, hydrogen, or ionic bonds) or through a tether molecule, such as a hydrophilic polymer or any such molecule described herein. In certain embodiments, the tether molecule is a hydrophilic polymer such as polyethylene glycol (PEG), polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and a hydrophilic peptide sequence. The PEG molecule may be between 500-10,000 Da (e.g., 1,000-5,000 Da such as 2,000 Da). In certain embodiments, the hydrophilic polymer is on the outer surface of the transport vector. The targeting polypeptide may be conjugated to the transport vector by any appropriate means, through covalent bonding (e.g., through a linker such as any of those described herein).

The transport vector may include any transport vector known in the art (e.g., those described herein). The transport vectors of the invention may include any lipid, carbohydrate, or polymer-based composition capable of transporting an agent (e.g., an agent such as those described herein). Transport vectors include lipid vectors (e.g., liposomes, micelles, polyplex, and lipoplexes) and polymer-based vectors such as dendrimers. Other transport vectors include nanoparticles, which can include silica, lipid, carbohydrate, or other pharmaceutically acceptable polymers. Transport vectors can protect against degradation of an agent (e.g., any described herein), thereby increasing the pharmacological half-life and bio-availability of these compounds.

The conjugation between the transport vector and the targeting polypeptide can take place using any linker described herein or known in the art.

In any of the above aspects, the transport vector may be bound to or may contain, or be capable of being bound to or containing, a therapeutic agent such as a nucleic acid (e.g., an RNAi agent or a nucleic acid encoding an RNAi agent), an anticancer agent, a polypeptide, or a hydrophobic agent, such as those described herein.

The polynucleotide may be a DNA molecule, an RNA molecule, a modified nucleic acid (e.g., containing nucleotide analogs), or a combination thereof. The polynucleotide may be single-stranded, double-stranded, linear, circular (e.g., a plasmid), nicked circular, coiled, supercoiled, concatemerized, or charged. Additionally, polynucleotides may contain 5′ and 3′ sense and antisense strand terminal modifications and can have blunt or overhanging terminal nucleotides, or combinations thereof. The polynucleotides can be an expression vector, a short interfering RNA (siRNA), short hairpin RNA (shRNA), double-stranded RNA (dsRNA), or microRNA (miRNA) molecule, or the nucleic acid can encode such molecules. In another embodiment, the siRNA, shRNA, dsRNA, or miRNA molecule of the invention has a nucleotide sequence with at least 70%, 80%, 90%, 95%, or 100% sequence identity, to any of the sequences set forth in SEQ ID NOS:117-129. The cancers and neurodegenerative diseases shown in Table 2 are amenable to treatment with RNAi agents; the lysosomal storage disorders can be treated by expression of the proteins indicated.

TABLE 2
Exemplary Diseases and Target Molecules

| Disease/Condition | Target Molecules |
|---|---|
| Cancer | |
| Glioblastoma | Epidermal growth factor receptor |
| | (EGFR), Vascular endothelial growth |
| | factor (VEGF) |
| Glioma | EGFR, VEGF |
| Astrocytoma | EGFR, VEGF |
| Neuroblastoma | EGFR, VEGF |
| Lung cancer | EGFR, VEGF |
| Breast cancer | EGFR, VEGF |
| Hepatocellular carcinoma | EGFR, VEGF |
| Neurodegenerative Disease | |
| Huntington's disease | Huntingtin (Htt) |
| Parkinson's disease | α-synuclein |
| Alzheimer's disease | Amyloid precursor protein (APP), |
| | Presenilin-1 or -2, Apolipoprotein E |
| | (ApoE) |
| Amyotropic lateral sclerosis | Superoxide dismutase 1 (SOD-1) |
| Multiple sclerosis | Sorting nexin-6 (SNX6), LINGO-1, |
| | Nogo-A, NgR-1, APP |
| Lysosomal Storage Disease | |
| MPS-I (Hurler, Scheie diseases) | α-L-iduronidase |
| MPS-II (Hunter syndrome) | Iduronate sulfatase |
| MPS-IIIA (Sanfilippo syndrome A) | Heparan N-sulfatase |
| MPS-IIIB (Sanfilippo syndrome B) | α-N-acetylglucosaminidase |
| MPS-IIIC (Sanfilippo syndrome C) | Acetyl-CoA:α-glucosaminide |
| | acetyltransferase |
| MPS-IIID (Sanfilippo syndrome D) | N-acetylglucosamine 6-sulfatase |
| MPS-VI (Maroteaux-Lamy | N-acetylgalactosamine 4-sulfatase |
| syndrome) | |
| MPS-VII (Sly syndrome) | β-glucuronidase |
| Niemann-Pick disease | Sphingomyelinase |
| Gaucher's disease | Glucocerebrosidase |
| Fabry disease | α-galactosidase-A |
| Farber's disease | Ceramidase |
| Krabbé disease | Galactosylceramidase |
| Metachromatic leukodystrophy | Arylsulfatase A |
| Alexander disease | Glial fibrillary acidic protein |
| Canavan disease | Aspartoacylase |
| Refsum's disease | Phytanoyl-CoA hydroxylase or |
| | peroxin-7 |
| GM1 gangliosidoses | β-galactosidase |
| GM2 gangliosidoses (e.g., Tay- | β-hexosaminidase A |
| Sachs, Sandhoff diseases) | |
| Aspartylglucosaminuria | Aspartylglucosaminidase (AGA). |
| Fucosidosis | Fucosidase |
| Mannosidosis | α-mannosidase |
| Mucolipodosis (sialidosis) | Sialidase |

The polypeptide may be a GLP-1 agonist (e.g., GLP-1, exendin-4, and analogs thereof), leptin, neurotensin, glial-derived neurotrophic factor (GDNF), brain-derived neurotrophic factor (BDNF), or an analog thereof (e.g., those described herein).

In certain embodiments, the transport vector is not a polyamidoamine dendrimer, the linker is not polyethylene glycol (e.g., PEG₃₄₀₀), and/or the targeting polypeptide is not SEQ ID NO:97, SEQ ID NO:74, and/or SEQ ID NO:113. In certain embodiments, the transport vector is not polyethyleneimine (PEI), poly(lactic-glycolic) acid (PLGA), and/or polylactic acid (PLA). In other embodiments, the transport vector is not made of polylactic acid, polyglycolic acid, or is not a hydrogel. In certain embodiments, the transport vector is not a liposome, a microemulsion, a micelle, a unilamellar or multilamellar vesicle, an erythrocyte ghost, or a spheroplasts.

By “blood-brain barrier” or “BBB” is meant the membranic structure that protects the brain from chemicals in the blood, while still allowing essential metabolic function. The BBB is composed of endothelial cells, which are packed very tightly in brain capillaries. The BBB includes the blood-retinal barrier.

By “cancer” or “proliferative disease” is meant cellular proliferation resulting from the loss of normal control, thereby resulting in unregulated growth, lack of differentiation, or the ability to invade local tissues and metastasize, or a combination thereof. Cancer can develop in any tissue, in any organ, or in any cell type.

By “fragment” is meant a polypeptide originating from a portion of an original or parent sequence or from an analog of said parent sequence. Fragments encompass polypeptides having truncations of one or more (e.g., 2, 3, 4, 5, 6, 7, 8, 9, 10, 11, 12, 13, 14, 15, 16, 17, 18, or 19) amino acids wherein the truncation may originate from the amino terminus (N-terminus), carboxy terminus (C-terminus), or from the interior of the protein.

By “analog” is meant a compound having structural similarity and retaining at least some activity of the parent molecule (e.g., at least 1%, 5%, 10%, 25%, 50%, 75%, 90%, or 95%). An analog of a polypeptide, for example, may be substantially identical to the parent polypeptide.

By “substantial identity” or “substantially identical” is meant a polypeptide or polynucleotide sequence that has the same polypeptide or polynucleotide sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least 50%, 60%, 70%, 75%, 80%, 85%, 90%, 95%, 96%, 97%, 98%, 99%, or 100% identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full length sequence). For polynucleotides, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

By “transport vector” is meant any compound or composition (e.g., lipid, carbohydrate, polymer, or surfactant) capable of binding or containing a therapeutic agent. The transport vector may be capable of transporting the agent, such as a small molecule therapeutic or polynucleotide. Exemplary transport vectors include lipid micelles, liposomes, lipoplexes, and dendrimers.

By “lysosomal storage disease” is meant any disorder that results from a defect in lysosomal function. Exemplary lysosomal storage diseases are the mucopolysaccharidoses (MPS, e.g., Hunter syndrome), leukodystrophies (e.g., metachromatic leukodystrophy), gangliosidoses (e.g., Tay-Sachs disease), mucolipidoses, lipidoses (e.g., Gaucher's disease), and glycoproteinoses. Additional lysosomal storage diseases are described herein.

By “modulate” is meant that the expression of a gene, or level of an RNA molecule or equivalent RNA molecules encoding one or more proteins or protein subunits, or activity of one or more proteins or protein subunits is up-regulated or down-regulated, such that expression, level, or activity is greater than or less than that observed in the absence of the modulator. For example, the term modulate can include inhibition.

By “neurodegenerative disease” is meant any disease or condition affecting the mammalian brain, CNS, the peripheral nervous system, or the autonomous nervous system wherein neurons are lost or deteriorate. Exemplary neurodegenerative diseases include Alzheimer's disease, Parkinson's disease, dementia with Lewy bodies, multiple system atrophy, Krabbé disease, multiple sclerosis, narcolepsy, and HIV-associated dementia. Other neurodegenerative diseases are described herein.

By “polypeptide” is meant any chain of amino acids, or analogs thereof, regardless of length or post-translational modification (for example, glycosylation or phosphorylation).

A “non-naturally occurring amino acid” is an amino acid not naturally produced or found in a mammal.

By “subject” is meant any human or non-human animal (e.g., a mammal).

By “providing” is meant, in the context of a conjugate of the invention, to bring the conjugate into contact with a target cell or tissue either in vivo or in vitro. A conjugate may be provided by administering the vector or conjugate to a subject.

By “RNAi agent” is meant any agent or compound that exerts a gene silencing effect through an RNA interference pathway. RNAi agents include polynucleotides that are capable of mediating sequence-specific RNAi, for example, a short interfering RNA (siRNA), double-stranded RNA (dsRNA), microRNA (miRNA), short hairpin RNA (shRNA), short interfering oligonucleotide, short interfering nucleic acid, short interfering modified oligonucleotide, chemically-modified siRNA, and post-transcriptional gene silencing RNA (ptgsRNA).

By “double-stranded RNA” (dsRNA) is meant a double-stranded RNA molecule that can be used to silence a gene product through RNA interference.

By “microRNA” (miRNA) is meant a single-stranded RNA molecule that can be used to silence a gene product through RNA interference.

By “short hairpin RNA” or “shRNA” is meant a sequence of RNA that makes a tight hairpin turn and is capable of gene silencing.

By “small inhibitory RNA,” “short interfering RNA,” or “siRNA” are meant a class of 10-40 (e.g., 15-25, such as 21) nucleotide double-stranded RNA molecules that are capable of gene silencing.

By “silencing” or “gene silencing” is meant that the expression of a gene or the level of an RNA molecule that encodes one or more proteins is reduced in the presence of an RNAi agent below that observed under control conditions (e.g., in the absence of the RNAi agent or in the presence of an inactive or attenuated molecule such as an RNAi molecule with scrambled sequence or with mismatches).

By “substantially pure” or “isolated” is meant a compound (e.g., a polypeptide or conjugate) that has been separated from other chemical components. Typically, the compound is substantially pure when it is at least 30%, by weight, free from other components. In certain embodiments, the preparation is at least 50%, 60%, 75%, 85%, 90%, 95%, 96%, 97%, 98%, or 99% by weight, free from other components. A purified polypeptide may be obtained, for example, by expression of a recombinant polynucleotide encoding such a polypeptide or by chemically synthesizing the polypeptide. Purity can be measured by any appropriate method, for example, column chromatography, polyacrylamide gel electrophoresis, or by HPLC analysis.

By “agent” is meant any compound, for example, an antibody, or a therapeutic agent, a detectable label (e.g., a marker, tracer, or imaging compound).

By “therapeutic agent” is meant any compound having a biological activity. Therapeutic agents may be useful for treating conditions or diseases.

By “tether molecule” is meant any molecule capable of chemically binding a targeting polypeptide to a transport vector. Exemplary tether molecules are described herein and include hydrophilic polymers and molecules such as DNA strands, actin filaments, and fibronectin.

“Treating” a disease or condition in a subject or “treating” a subject having a disease or condition refers to subjecting the individual to a pharmaceutical treatment, e.g., the administration of a drug, such that at least one symptom of the disease or condition is decreased or stabilized.

By “treating prophyllactically” a disease or condition in a subject is meant reducing or eliminating the risk of developing (i.e., the incidence) of or reducing the severity of the disease or condition prior to the appearance of at least one symptom of the disease.

By “treating cancer,” “preventing cancer,” or “inhibiting cancer” is meant causing a reduction in the size of a tumor or the number of cancer cells, slowing, preventing, or inhibiting an increase in the size of a tumor or cancer cell proliferation, increasing the disease-free survival time between the disappearance of a tumor or other cancer and its reappearance, preventing or reducing the likelihood of an initial or subsequent occurrence of a tumor or other cancer, or reducing an adverse symptom associated with a tumor or other cancer.

By a polypeptide or conjugate which is “efficiently transported across the BBB” is meant a polypeptide that is able to cross the BBB at least as efficiently as Angiopep-6 (i.e., greater than 38.5% that of Angiopep-1 (250 nM) in the in situ brain perfusion assay described in U.S. Patent Application Publication No. 2009/0016959, hereby incorporated by reference). Accordingly, a vector or conjugate which is “not efficiently transported across the BBB” is transported to the brain at lower levels (e.g., transported less efficiently than Angiopep-6).

By a polypeptide or conjugate which is “efficiently transported to a particular cell type” is meant that the polypeptide or conjugate is able to accumulate (e.g., either due to increased transport into the cell, decreased efflux from the cell, or a combination thereof) in that cell type to at least a 10% (e.g., 25%, 50%, 100%, 200%, 500%, 1,000%, 5,000%, or 10,000%) greater extent than either a control substance, or, in the case of a conjugate, as compared to the unconjugated agent or transport vector. Such activities are described in detail in International Application Publication No. WO 2007/009229, hereby incorporated by reference.

### DETAILED DESCRIPTION OF THE INVENTION

The present invention features a conjugate between a targeting polypeptide and a transport vector. The targeting polypeptide is capable of directing the transport vector into the brain, into the central nervous system (CNS), or into other cells, tissues, and organs. Typically, the transport vector will be bound to or will contain a therapeutic agent. The therapeutic agent may be any agent known in the art (e.g., those described herein). Agents include small molecules, polypeptides, and polynucleotides, such as RNA interference (RNAi) agents or polynucleotides encoding an RNAi agent. The transport vector, in certain embodiments, can stabilize, protect (e.g., nuclease protection), or assist in targeting the agent to a desired tissue or cell. In one example, polypeptide-transport vectors carrying an RNAi agent can target the agent to the brain of an individual in need of treatment. In addition, other agents that are unable or ineffective at crossing the blood-brain barrier (BBB) by themselves can be transported across the BBB when carried by a polypeptide-transport vector. Such polypeptide-transport vector conjugates can be used to treat conditions or diseases such as cancer, neurodegenerative conditions, and lysosomal storage disorders.

### Targeting Polypeptides

The conjugates of the invention feature a targeting polypeptide. Such polypeptides are described herein and in U.S. Pat. No. 7,557,182 and include any of the peptides described in Table 1 (e.g., Angiopep-1 or Angiopep-2), or a fragment or analog thereof. In certain embodiments, the targeting polypeptide may have at least 35%, 40%, 50%, 60%, 70%, 80%, 90%, 95%, 99%, or even 100% identity to a polypeptide of Table 1. The targeting polypeptide may have one or more (e.g., 2, 3, 4, 5, 6, 7, 8, 9, 10, 11, 12, 13, 14, or 15) substitutions relative to one of these sequences. Other modifications are described in greater detail below.

The targeting polypeptide can also be a fragment of the polypeptide described herein (e.g., a functional fragment). In certain embodiments, the fragments are capable of efficiently being transported to or accumulating in a particular cell type (e.g., liver, eye, lung, kidney, or spleen) or are efficiently transported across the BBB. Truncations of the polypeptide may be 1, 2, 3, 4, 5, 6, 7, 8, 9, 10, 11, 12, or more amino acids from either the N-terminus of the polypeptide, the C-terminus of the polypeptide, or a combination thereof. Other fragments include sequences where internal portions of the polypeptide are deleted.

Additional targeting polypeptides may be identified by using one of the assays or methods described herein. For example, a candidate polypeptide may be produced by conventional peptide synthesis, conjugated with paclitaxel and administered to a laboratory animal. A biologically active polypeptide conjugate may be identified, for example, based on its ability to increase survival of an animal injected with tumor cells and treated with the conjugate as compared to a control which has not been treated with a conjugate (e.g., treated with the unconjugated agent). For example, a biologically active polypeptide may be identified based on its location in the parenchyma in an in situ cerebral perfusion assay.

Assays to determine accumulation in other tissues may be performed as well. Labeled conjugates of a polypeptide can be administered to an animal, and accumulation in different organs can be measured. For example, a polypeptide conjugated to a detectable label (e.g., a near-IR fluorescence spectroscopy label such as Cy5.5) allows live in vivo visualization. Such a polypeptide can be administered to an animal, and the presence of the polypeptide in an organ can be detected, thus allowing determination of the rate and amount of accumulation of the polypeptide in the desired organ. In other embodiments, the polypeptide can be labelled with a radioactive isotope (e.g., ¹²⁵I). The polypeptide is then administered to an animal. After a period of time, the animal is sacrificed and the organs are extracted. The amount of radioisotope in each organ can then be measured using any means known in the art. By comparing the amount of a labeled candidate polypeptide in a particular organ relative to the amount of a labeled control polypeptide, the ability of the candidate polypeptide to access and accumulate in a particular tissue can be ascertained. Appropriate negative controls include any polypeptide known not to be efficiently transported into a particular cell type (e.g., a polypeptide related to Angiopep that does not cross the BBB, or any other polypeptide).

Additional sequences are described in U.S. Pat. No. 5,807,980 (e.g., SEQ ID NO:102 herein), U.S. Pat. No. 5,780,265 (e.g., SEQ ID NO:103), U.S. Pat. No. 5,118,668 (e.g., SEQ ID NO:105). An exemplary nucleotide sequence encoding an aprotinin analog atgagaccag atttctgcct cgagccgccg tacactgggc cctgcaaagc tcgtatcatc cgttacttct acaatgcaaa ggcaggcctg tgtcagacct tcgtatacgg cggctgcaga gctaagcgta acaacttcaa atccgcggaa gactgcatgc gtacttgcgg tggtgettag (SEQ ID NO:106; Genbank accession No. X04666). Other examples of aprotinin analogs may be found by performing a protein BLAST (Genbank: www.ncbi.nlm.nih.gov/BLAST/) using the synthetic aprotinin sequence (or portion thereof) disclosed in PCT Publication No. WO 2004/060403. Exemplary aprotinin analogs are also found under accession Nos. CAA37967 (GI:58005) and 1405218C (GI:3604747).

Modified Polypeptides

The targeting polypeptides used in the invention (e.g., a polypeptide having a sequence described in any one of SEQ ID NOS:1-93, 97-105 and 107-116 such as Angiopep-1 (SEQ ID NO:67) or Angiopep-2 (SEQ ID NO:97)), as well as the biological active (e.g., therapeutic) polypeptide described herein, may have a modified amino acid sequence. In certain embodiments, the modification does not destroy significantly a desired biological activity. The modification may reduce (e.g., by at least 5%, 10%, 20%, 25%, 35%, 50%, 60%, 70%, 75%, 80%, 90%, or 95%), may have no effect, or may increase (e.g., by at least 5%, 10%, 25%, 50%, 100%, 200%, 500%, or 1000%) the biological activity of the original polypeptide. The modified polypeptide may have or may optimize a characteristic of a polypeptide, such as in vivo stability, bioavailability, toxicity, immunological activity, immunological identity, and conjugation properties.

Modifications include those by natural processes, such as posttranslational processing, or by chemical modification techniques known in the art. Modifications may occur anywhere in a polypeptide including the polypeptide backbone, the amino acid side chains and the amino- or carboxy-terminus. The same type of modification may be present in the same or varying degrees at several sites in a given polypeptide, and a polypeptide may contain more than one type of modification. Polypeptides may be branched as a result of ubiquitination, and they may be cyclic, with or without branching. Cyclic, branched, and branched cyclic polypeptides may result from posttranslational natural processes or may be made synthetically. Other modifications include pegylation, acetylation, acylation, addition of acetomidomethyl (Acm) group, ADP-ribosylation, alkylation, amidation, biotinylation, carbamoylation, carboxyethylation, esterification, covalent attachment to fiavin, covalent attachment to a heme moiety, covalent attachment of a nucleotide or nucleotide derivative, covalent attachment of drug, covalent attachment of a marker (e.g., fluorescent or radioactive), covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination.

A modified polypeptide can also include an amino acid insertion, deletion, or substitution, either conservative or non-conservative (e.g., D-amino acids, desamino acids) in the polypeptide sequence (e.g., where such changes do not substantially alter the biological activity of the polypeptide). In particular, the addition of one or more cysteine residues to the amino or carboxy terminus of any of the polypeptides described herein can facilitate conjugation of these polypeptides by, e.g., disulfide bonding. For example, Angiopep-1 (SEQ ID NO:67), Angiopep-2 (SEQ ID NO:97), or Angiopep-7 (SEQ ID NO:112) can be modified to include a single cysteine residue at the amino-terminus (SEQ ID NOS: 71, 113, and 115, respectively) or a single cysteine residue at the carboxy-terminus (SEQ ID NOS: 72, 114, and 116, respectively). Amino acid substitutions can be conservative (i.e., wherein a residue is replaced by another of the same general type or group) or non-conservative (i.e., wherein a residue is replaced by an amino acid of another type). In addition, a non-naturally occurring amino acid can be substituted for a naturally occurring amino acid (i.e., non-naturally occurring conservative amino acid substitution or a non-naturally occurring non-conservative amino acid substitution).

Polypeptides made synthetically can include substitutions of amino acids not naturally encoded by DNA (e.g., non-naturally occurring or unnatural amino acid). Examples of non-naturally occurring amino acids include D-amino acids, an amino acid having an acetylaminomethyl group attached to a sulfur atom of a cysteine, a pegylated amino acid, the omega amino acids of the formula NH₂(CH₂)ₙCOOH wherein n is 2-6, neutral nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties.

Analogs may be generated by substitutional mutagenesis and retain the biological activity of the original polypeptide. Examples of substitutions identified as “conservative substitutions” are shown in Table 3. If such substitutions result in a change not desired, then other type of substitutions, denominated “exemplary substitutions” in Table 3, or as further described herein in reference to amino acid classes, are introduced and the products screened.

Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:

(1) hydrophobic: norleucine, methionine (Met), Alanine (Ala), Valine (Val), Leucine (Leu), Isoleucine (Ile), Histidine (His), Tryptophan (Trp), Tyrosine (Tyr), Phenylalanine (Phe),

(2) neutral hydrophilic: Cysteine (Cys), Serine (Ser), Threonine (Thr)

(3) acidic/negatively charged: Aspartic acid (Asp), Glutamic acid (Glu)

(4) basic: Asparagine (Asn), Glutamine (Gin), Histidine (His), Lysine (Lys), Arginine (Arg)

(5) residues that influence chain orientation: Glycine (Gly), Proline (Pro);

(6) aromatic: Tryptophan (Trp), Tyrosine (Tyr), Phenylalanine (Phe), Histidine (His),

(7) polar: Ser, Thr, Asn, Gln

(8) basic positively charged: Arg, Lys, His, and;

(9) charged: Asp, Glu, Arg, Lys, H is

Other amino acid substitutions are listed in Table 3.

TABLE 3
Amino acid substitutions

| Original | | Conservative |
|---|---|---|
| residue | Exemplary substitution | substitution |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, Ala, | Ile |
| | Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

Polypeptide Derivatives and Peptidomimetics

In addition to polypeptides consisting of naturally occurring amino acids, peptidomimetics or peptide analogs are also encompassed by the present invention. Peptide analogs are commonly used in the pharmaceutical industry as non-peptide drugs with properties analogous to those of the template polypeptide. The non-peptide compounds are termed “peptide mimetics” or peptidomimetics (Fauchere et al., Infect. Immun. 54:283-287, 1986 and Evans et al., J. Med. Chem. 30:1229-1239, 1987). Peptide mimetics that are structurally related to therapeutically useful peptides or polypeptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1:267, 1983; Spatola et al., Life Sci. 38:1243-1249, 1986; Hudson et al., Int. J. Pept. Res. 14:177-185, 1979; and Weinstein, 1983, Chemistry and Biochemistry, of Amino Acids, Peptides and Proteins, Weinstein eds, Marcel Dekker, New York). Such peptide mimetics may have significant advantages over naturally occurring polypeptides including more economical production, greater chemical stability, enhanced pharmacological properties (e.g., half-life, absorption, potency, efficiency), reduced antigenicity, and others.

While the polypeptides described herein can efficiently cross the BBB or enter particular cell types (e.g., those described herein), their effectiveness may be reduced by the presence of proteases. Serum proteases have specific substrate requirements, including L-amino acids and peptide bonds for cleavage. Furthermore, exopeptidases, which represent the most prominent component of the protease activity in serum, usually act on the first peptide bond of the polypeptide and require a free N-terminus (Powell et al., Pharm. Res. 10:1268-1273, 1993). In light of this, it is often advantageous to use modified versions of polypeptides. The modified polypeptides retain the structural characteristics of the original L-amino acid polypeptides, but advantageously are not readily susceptible to cleavage by protease and/or exopeptidases.

Systematic substitution of one or more amino acids of a consensus sequence with D-amino acid of the same type (e.g., an enantiomer; D-lysine in place of L-lysine) may be used to generate more stable polypeptides. Thus, a polypeptide derivative or peptidomimetic as described herein may be all L-, all D-, or mixed D, L polypeptides. The presence of an N-terminal or C-terminal D-amino acid increases the in vivo stability of a polypeptide because peptidases cannot utilize a D-amino acid as a substrate (Powell et al., Pharm. Res. 10:1268-1273, 1993). Reverse-D polypeptides are polypeptides containing D-amino acids, arranged in a reverse sequence relative to a polypeptide containing L-amino acids. Thus, the C-terminal residue of an L-amino acid polypeptide becomes N-terminal for the D-amino acid polypeptide, and so forth. Reverse D-polypeptides retain the same tertiary conformation and therefore the same activity, as the L-amino acid polypeptides, but are more stable to enzymatic degradation in vitro and in vivo, and thus have greater therapeutic efficacy than the original polypeptide (Brady and Dodson, Nature 368:692-693, 1994 and Jameson et al., Nature 368:744-746, 1994). In addition to reverse-D-polypeptides, constrained polypeptides comprising a consensus sequence or a substantially identical consensus sequence variation may be generated by methods well known in the art (Rizo et al., Ann. Rev. Biochem. 61:387-418, 1992). For example, constrained polypeptides may be generated by adding cysteine residues capable of forming disulfide bridges and, thereby, resulting in a cyclic polypeptide. Cyclic polypeptides have no free N- or C-termini. Accordingly, they are not susceptible to proteolysis by exopeptidases, although they are, of course, susceptible to endopeptidases, which do not cleave at polypeptide termini. The amino acid sequences of the polypeptides with N-terminal or C-terminal D-amino acids and of the cyclic polypeptides are usually identical to the sequences of the polypeptides to which they correspond, except for the presence of N-terminal or C-terminal D-amino acid residue, or their circular structure, respectively.

A cyclic derivative containing an intramolecular disulfide bond may be prepared by conventional solid phase synthesis while incorporating suitable S-protected cysteine or homocysteine residues at the positions selected for cyclization such as the amino and carboxy termini (Sah et al., J. Pharm. Pharmacol. 48:197, 1996). Following completion of the chain assembly, cyclization can be performed either (1) by selective removal of the S-protecting group with a consequent on-support oxidation of the corresponding two free SH-functions, to form a S—S bonds, followed by conventional removal of the product from the support and appropriate purification procedure or (2) by removal of the polypeptide from the support along with complete side chain de-protection, followed by oxidation of the free SH-functions in highly dilute aqueous solution.

The cyclic derivative containing an intramolecular amide bond may be prepared by conventional solid phase synthesis while incorporating suitable amino and carboxyl side chain protected amino acid derivatives, at the position selected for cyclization. The cyclic derivatives containing intramolecular —S-alkyl bonds can be prepared by conventional solid phase chemistry while incorporating an amino acid residue with a suitable amino-protected side chain, and a suitable S-protected cysteine or homocysteine residue at the position selected for cyclization.

Another effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharm. Res. 10:1268-1273, 1993). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified polypeptides consisting of polypeptides bearing an N-terminal acetyl group and/or a C-terminal amide group.

Also included by the present invention are other types of polypeptide derivatives containing additional chemical moieties not normally part of the polypeptide, provided that the derivative retains the desired functional activity of the polypeptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives: (5) derivatives conjugated to an antibody or other biological ligand; and (6) other types of derivatives.

Longer polypeptide sequences which result from the addition of additional amino acid residues to the polypeptides described herein are also encompassed in the present invention. Such longer polypeptide sequences can be expected to have the same biological activity and specificity (e.g., cell tropism) as the polypeptides described above. While polypeptides having a substantial number of additional amino acids are not excluded, it is recognized that some large polypeptides may assume a configuration that masks the effective sequence, thereby preventing binding to a target (e.g., a member of the LRP receptor family such as LRP or LRP2). These derivatives could act as competitive antagonists. Thus, while the present invention encompasses polypeptides or derivatives of the polypeptides described herein having an extension, desirably the extension does not destroy the cell targeting activity of the polypeptides or its derivatives.

Other derivatives included in the present invention are dual polypeptides consisting of two of the same, or two different polypeptides, as described herein, covalently linked to one another either directly or through a spacer, such as by a short stretch of alanine residues or by a putative site for proteolysis (e.g., by cathepsin, see e.g., U.S. Pat. No. 5,126,249 and European Patent No. 495 049). Multimers of the polypeptides described herein consist of a polymer of molecules formed from the same or different polypeptides or derivatives thereof.

The present invention also encompasses polypeptide derivatives that are chimeric or fusion proteins containing a polypeptide described herein, or fragment thereof, linked at its amino- or carboxy-terminal end, or both, to an amino acid sequence of a different protein. Such a chimeric or fusion protein may be produced by recombinant expression of a polynucleotide encoding the protein. For example, a chimeric or fusion protein may contain at least 6 amino acids shared with one of the described polypeptides which desirably results in a chimeric or fusion protein that has an equivalent or greater functional activity.

Assays to Identify Peptidomimetics

As described above, non-peptidyl compounds generated to replicate the backbone geometry and pharmacophore display (peptidomimetics) of the polypeptides described herein often possess attributes of greater metabolic stability, higher potency, longer duration of action, and better bioavailability.

Peptidomimetics compounds can be obtained using any of the numerous approaches in combinatorial library methods known in the art, including biological libraries, spatially addressable parallel solid phase or solution phase libraries, synthetic library methods requiring deconvolution, the ‘one-bead one-compound’ library method, and synthetic library methods using affinity chromatography selection. The biological library approach is limited to peptide libraries, while the other four approaches are applicable to peptide, non-peptide oligomer, or small molecule libraries of compounds (Lam, Anticancer Drug Des. 12:145, 1997). Examples of methods for the synthesis of molecular libraries can be found in the art, for example, in: DeWitt et al. (Proc. Natl. Acad. Sci. USA 90:6909, 1993); Erb et al. (Proc. Natl. Acad. Sci. USA 91:11422, 1994); Zuckermann et al. (J. Med. Chem. 37:2678, 1994); Cho et al. (Science 261:1303, 1993); Carell et al. (Angew. Chem., Int. Ed. Engl. 33:2059, 1994 and ibid 2061); and Gallop et al. (Med. Chem. 37:1233, 1994). Libraries of compounds may be presented in solution (e.g., Houghten, Biotechniques 13:412-421, 1992) or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. USA 89:1865-1869, 1992) or on phage (Scott and Smith, Science 249:386-390, 1990), or luciferase, and the enzymatic label detected by determination of conversion of an appropriate substrate to product.

Once a polypeptide as described herein is identified, it can be isolated and purified by any number of standard methods including, but not limited to, differential solubility (e.g., precipitation), centrifugation, chromatography (e.g., affinity, ion exchange, and size exclusion), or by any other standard techniques used for the purification of peptides, peptidomimetics, or proteins. The functional properties of an identified polypeptide of interest may be evaluated using any functional assay known in the art. Desirably, assays for evaluating downstream receptor function in intracellular signaling are used (e.g., cell proliferation).

For example, the peptidomimetics compounds of the present invention may be obtained using the following three-phase process: (1) scanning the polypeptides described herein to identify regions of secondary structure necessary for targeting the particular cell types described herein; (2) using conformationally constrained dipeptide surrogates to refine the backbone geometry and provide organic platforms corresponding to these surrogates; and (3) using the best organic platforms to display organic pharmocophores in libraries of candidates designed to mimic the desired activity of the native polypeptide. In more detail the three phases are as follows. In phase 1, the lead candidate polypeptides are scanned and their structure abridged to identify the requirements for their activity. A series of polypeptide analogs of the original are synthesized. In phase 2, the best polypeptide analogs are investigated using the conformationally constrained dipeptide surrogates. Indolizidin-2-one, indolizidin-9-one and quinolizidinone amino acids (I²aa, I⁹aa and Qaa respectively) are used as platforms for studying backbone geometry of the best peptide candidates. These and related platforms (reviewed in Halab et al., Biopolymers 55:101-122, 2000 and IIanessian et al., Tetrahedron 53:12789-12854, 1997) may be introduced at specific regions of the polypeptide to orient the pharmacophores in different directions. Biological evaluation of these analogs identifies improved lead polypeptides that mimic the geometric requirements for activity. In phase 3, the platforms from the most active lead polypeptides are used to display organic surrogates of the pharmacophores responsible for activity of the native peptide. The pharmacophores and scaffolds are combined in a parallel synthesis format. Derivation of polypeptides and the above phases can be accomplished by other means using methods known in the art.

Structure function relationships determined from the polypeptides, polypeptide derivatives, peptidomimetics or other small molecules described herein may be used to refine and prepare analogous molecular structures having similar or better properties. Accordingly, the compounds of the present invention also include molecules that share the structure, polarity, charge characteristics and side chain properties of the polypeptides described herein.

In summary, based on the disclosure herein, those skilled in the art can develop peptides and peptidomimetics screening assays which are useful for identifying compounds for targeting an agent to particular cell types (e.g., those described herein). The assays of this invention may be developed for low-throughput, high-throughput, or ultra-high throughput screening formats. Assays of the present invention include assays amenable to automation.

### Transport Vectors

The transport vectors of the invention may include any lipid, carbohydrate, or polymer-based composition capable of transporting an agent (e.g., an agent such as those described herein). Transport vectors include lipid vectors (e.g., liposomes, micelles, and polyplexes) and polymer-based vectors such as dendrimers. Other transport vectors include nanoparticles, which can include silica, lipid, carbohydrate, or other pharmaceutically-acceptable polymers. Transport vectors can protect against degradation of an agent (e.g., any described herein), thereby increasing the pharmacological half-life and bio-availability of these compounds.

Lipid Vectors

Lipid vectors can be formed using any biocompatible lipid or combination of lipids capable for forming lipid vectors (e.g., liposomes, micelles, and lipoplexes). Encapsulation of an agent into a lipid vector can protect the agent from damage or degradation or facilitate its entry into a cell. Lipid vectors, as a result of charge interactions (e.g., a cationic lipid vector and anionic cell membrane), interact and fuse with the cell membrane, thus releasing the agent into the cytoplasm. A liposome is a bilayered vesicle comprising one or more of lipid molecules, polypeptide-lipid conjugates, and lipid components. A lipoplex is a liposome formed with cationic lipid molecules to impart an overall positive charge to the liposome. A micelle is vesicle with a single layer of surfactants or lipid molecules.

Liposomes

In certain embodiments, the lipid vector is a liposome. Typically, the lipids used are capable of forming a bilayer and are cationic. Classes of suitable lipid molecules include phospholipids (e.g., phosphotidylcholine), fatty acids, glycolipids, ceramides, glycerides, and cholesterols, or any combination thereof. Alternatively or in addition, the lipid vector can include neutral lipids (e.g., dioleoylphosphatidyl ethanolamine (DOPE)). Other lipids that can form lipid vectors are known in the art and described herein.

As used herein, a “lipid molecule” is a molecule with a hydrophobic head moiety and a hydrophilic tail moiety and may be capable of forming liposomes. The lipid molecule can optionally be modified to include hydrophilic polymer groups. Examples of such lipid molecules include 1,2-distearoyl-sn-glycero-3-phosphoethanolamine-N—[methoxy(polyethylene glycol)-2000] and 1,2-distearoyl-sn-glycero-3-phosphoethanolamine-N4-carboxy(polyethylene glycol)-20001.

Examples of lipid molecules include natural lipids, such as cardiolipin (CL), phosphatidic acid (PA), phosphatidylcholine (PC), phosphatidylethanolamine (PE), phosphatidylglycerol (PG), phosphatidylinositol (PI), and phosphatidyl serine (PS); sphingolipids, such as sphingosine, ceramide, sphingomyelin, cerebrosides, sulfatides, gangliosides, and phytosphingosine; cationic lipids, such as 1,2-dioleoyl-3-trimethylammonium-propane (DOTAP), 1,2-dioleoyl-3-dimethylammonium-propane (DODAP), dimethyldioctadecyl ammonium bromide (DDAB), 3-β-[N—(N′,N′-dimethylaminoethane)carbamoly]cholesterol (DC-Chol), N-[1-(2,3,-ditetradecyloxy)propyl]-N,N-dimethyl-N-hydroxyethylammonium bromide (DMRIE), N-[1-(2,3,-dioleyloxy)propyl]-N,N-dimethyl-N-hydroxy ethylammonium bromide (DORIE), and 1,2-di-O-octadecenyl-3-trimethylammonium propane (DOTMA); phosphatidylcholines, such as 1,2-dilauroyl-sn-glycero-3-ethylphosphocholine, 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), and 1-palmitoyl-2-oleoyl-sn-glycerol-3-phosphocholine (POPC); phosphoethanolamines, such as 1,2-dibutyryl-sn-glycero-3-phosphoethanolamine, 1,2-distearoyl-sn-glycero-3-phosphoethanolamine (DSPE), 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE), and 1,2-dioleoyl-sn-glycero-3-phosphoethanolamine-N-(glutaryl); phosphatidic acids, such as 1,2-dimyristoyl-sn-glycero-3-phosphate, 1,2-dipalmitoyl-sn-glycero-3-phosphate, and 1,2-dioleoyl-sn-glycero-3-phosphate; phosphatidylglycerols, such as dipalmitoyl phosphatidylglycerol (DMPC), 1,2-dimyristoyl-sn-glycero-3-phospho-(1′-rac-glycerol), and 1,2-dioleoyl-sn-glycero-3-phospho-(1′-rac-glycerol); phosphatidylserines, such as 1,2-dimyristoyl-sn-glycero-3-phospho-L-serine, 1,2-dipalmitoyl-sn-glycero-3-phospho-L-serine, and 1,2-dioleoyl-sn-glycero-3-phospho-L-serine; cardiolipins, such as 1′,3′-bis[1,2-d]myristoyl-sn-glycero-3-phospho]-sn-glycerol; and PEG-lipid conjugates, such as 1,2-dipalmitoyl-sn-glycero-3-phosphoethanolamine-N-[methoxy(polyethylene glycol)-750], 1,2-dipalmitoyl-sn-glycero-3-phosphoethanolamine-N-[methoxy(polyethylene glycol)-2000], 1,2-dipalmitoyl-sn-glycero-3-phosphoethanolamine-N-[methoxy(polyethylene glycol)-5000], 1,2-distearoyl-sn-glycero-3-phosphoethanolamine-N-[methoxy(polyethylene glycol)-2000], and 1,2-distearoyl-sn-glycero-3-phosphoethanolamine-N—[carboxy(polyethylene glycol)-2000].

Commercially available lipid compositions include Lipofectamine™ 2000 and Lipofectin® from Invitrogen Corp.; Transfectam® and Transfast™ from Promega Corp.; NeuroPORTER™ and Escort™ from Sigma-Aldrich Co.; FuGENE® 6 from Roche; and LipoTAXI® from Strategene. Known lipid compositions include the Trojan Horse Lipsome technology, as described in Boado, Pharm. Res. 24:1772-1787, 2007.

The liposomes can also include other components that aid in the formation or stability of liposomes. Examples of components include cholesterol, antioxidants (e.g., α-tocopherol, β-hydroxytoluidine), surfactants, and salts.

As used herein, a “polypeptide-lipid conjugate” is a lipid molecule that is bound to a targeting polypeptide by a covalent bond or a non-covalent bond (e.g., ionic interaction, entrapment or physical encapsulation, hydrogen bonding, absorption, adsorption, van der Waals forces, or any combinations thereof) with or without the use of a linker molecule.

The liposome can be of any useful combination comprising lipid molecules, including polypeptide-lipid conjugates and other components that aid in the formation or stability of liposomes. A person of skill in that art will know how to optimize the combination that favor encapsulation of a particular agent, stability of the liposome, scaled-up reaction conditions, or any other pertinent factor. Exemplary combinations are described in Boado, Pharm. Res. 24:1772-1787, 2007. In one example, the liposome comprises 93% POPC, 3% DDAB, 3% distearoylphosphatidylethanolamine (DSPE)-PEG2000, and 1% DSPE-PEG2000 covalently linked to a targeting polypeptide.

Producing liposomes typically occur through a general two-step process. In the first step, the lipids and lipid components are mixed in a volatile organic solvent or mixtures of solvents to ensure a homogenous mixture of lipids. Examples of solvents include chloroform, methanol, cyclohxane, and t-butanol. The solvent is then removed to form a dry lipid mixture in a film, powder, or pellet. The solvent can also be removed by using any known analytical techniques, such as by using nitrogen, rotary evaporation, spray drying, lyophilization, and vacuum-drying.

In the second step, the dry lipid mixture is hydrated with an aqueous solution to form liposomes. The agent can be added to the aqueous solution, which results in the formation of liposomes with encapsulated agent. Alternatively, the liposomes are first formed with a first aqueous solution and then exposed to another aqueous solution containing the agent. Encapsulation of the agent can be promoted by any known technique, such as by repeat freeze-thaw cycles, sonication, or mixing. A further example of this approach is described in Boado, Pharm. Res. 24:1772-1787, 2007. Alternatively, the agent is coupled to a hydrophobic moiety (e.g., cholesterol) to produce a lipophilic derivative and the lipophilic derivative is used with other lipid molecules to from liposomes.

During the second step, the dry lipid mixture may or may not contain the polypeptide-lipid conjugate. The process can optionally include various additional steps, including heating the aqueous solution past the phase transition temperature of the lipid molecules before adding it to the dry lipid mixture, where particular ranges of temperatures include from about 40° C. to about 70° C.; incubating the combination of the dry lipid mixture and the aqueous solution, where particular time ranges include from about 30 minutes to about 2 hours; mixing of the dry lipid mixture and the aqueous solution during incubation, such as by vortex mixing, shaking, stirring, or agitation; addition of nonelectrolytes to the aqueous solution to ensure physiological osmolality, such as a solution of 0.9% saline, 5% dextrose, and 10% sucrose; disruption of large multilamellar vesicles, such as by extrusion or sonication; and additional incubation of the pre-formed liposomes with polypeptide-lipid conjugate, where the dry lipid mixture did not contain the lipid molecules. One of skill in the art will be able to identify the particular temperature and incubation times during this hydration step to ensure incorporation of the derivatized lipid molecule into the liposomes or to obtain stable liposomes.

The polypeptide-lipid conjugate can be added at any point in the process of forming liposomes. In one example, the polypeptide-lipid conjugate is added to the lipids and lipid components during the formation of the dry lipid mixture. In another example, the polypeptide-lipid conjugate is added to liposomes that are pre-formed with a dry lipid mixture containing the lipids and lipid components. In yet another example, micelles are formed with the polypeptide-lipid conjugate, liposomes are formed with a dry lipid mixture containing lipids and lipid components, and then the micelles and liposomes are incubated together. The aqueous solution can include additional components to stabilize the agent or the liposome, such as buffers, salts, chelating agents, saline, dextrose, sucrose, etc.

In one example of this procedure, a dry film composed of the lipid mixture is hydrated with an aqueous solution containing an agent. This mixture is first heated to 50° C. for 30 minutes and then cooled to room temperature. Next, the mixture is transferred onto a dry film containing the polypeptide-lipid conjugate. The mixture is then incubated at 37° C. for two hours to incorporate the polypeptide-lipid conjugate into the liposomes containing the agent. See, e.g., Zhang et al., J. Control. Release 112:229-239, 2006.

Polyplexes

Complexes of polymers with agents are called polyplexes. Polyplexes typically consist of cationic polymers and their production is regulated by ionic interactions with an anionic agent (e.g., a polynucleotide). In some cases, polyplexes cannot release the bound agent into the cytoplasm. To this end, co-transfection with endosome-lytic agents (to lyse the endosome that is made during endocytosis) such as inactivated adenovirus must occur. In certain cases, polymers, such as polyethylenimine, have their own method of endosome disruption, as does chitosan and trimethylchitosan. Polyplexes are described, for example, in U.S. Patent Application Publication Nos. 2002/0009491; 2003/0134420; and 2004/0176282.

Polyplexes can be formed with any polymer and copolymer described herein, where non-charged or anionic polymers can be further derivatized to include cationic side chains. Examples of cationic side chains are amines, which are typically protonated under physiological conditions. Exemplary polymers that can be used to form polyplexes include polyamines, such as polylysine, polyarginine, polyamidoamine, and polyethylene imine.

Dendrimers

A dendrimer is a highly branched macromolecule with a spherical shape. The surface of the particle may be functionalized in many ways and many of the properties of the resulting construct are determined by its surface. In particular, it is possible to construct a cationic dendrimer (i.e., one with a positive surface charge). When in the presence of genetic material such as DNA or RNA, charge complimentarity leads to a temporary association of the polynucleotide with the cationic dendrimer. On reaching its destination the dendrimer-polynucleotide complex is then taken into the cell via endocytosis or across the BBB by transcytosis. Dendrimers are described, for example, in U.S. Pat. Nos. 6,113,946 and 7,261,875.

Dendrimers can be produced by any process known in the art. Under the divergent method, the core of the dendrimer is built first and successive steps build outward from the core to form branched structures. Under the convergent method, wedges of the dendrimer (or dendrons) are built separately, where successive steps build inward from the molecules that will make up the outer surface of the dendrimer. The different dendrons can be formed with the same or different polymeric monomers. Then, the dendrons are covalent linked to a core molecule or structure to form the dendrimer. Further examples of these methods are described in Svenson et al., Adv. Drug. Deliv. Rev. 57:2106-2129, 2005.

For polyamidoamine (PAMAM) dendrimers, the core of the dendrimer typically comprises an amino group. Exemplary core molecules include ammonia; diamine molecules, such as ethylenediamine, 1,4-diaminobutane, 1,6-diaminohexane, 1,12-diaminododecane, and cystamine; and triamine molecules, such as triethanolamine. In the first step of the addition reaction, polymeric monomers are used to build upon the core by reacting the monomers with the amino groups of the core to form a tetra-branched molecule. Subsequent addition reactions with the diamine molecule and the polymeric monomer further build upon the dendrimer.

Examples of polymeric monomers that react with amino groups include methacrylate to form PAMAM dendrimers; and acrylonitrile to form poly(propylene imine) dendrimers. Examples of PAMAM dendrimers and synthetic reactions of dendrimers are set forth in U.S. Pat. Nos. 4,507,466, 5,527,524, and 5,714,166. Examples of PAMAM dendrimers formed with a triethanolamine core are set forth in Wu et al., Chem. Comm. 3:313-315, 2005; and Zhou et al., Chem. Comm. 22:2362-2364, 2006. Synthesis of the dendrimers can include additional steps, such as adding protecting groups to activated groups in order to prevent intramolecular reactions; and adding a deprotection step to remove protecting groups.

In addition to PAMAM dendrimers, other types of dendrimers can be used. For phosphorous dendrimers, the core of the dendrimer comprises a P=0 group. Exemplary core molecules include a cyclotriphosphazene group and a thiophosphoryl group. Examples of polymeric monomers include phenoxymethyl(methylhydrazono) groups. Alternatively, the dendrimer is a hyperbranched polymer with a polyester core structure. Examples of such dendrimers include hyperbranched 2,2-bis(hydroxymethyl)propionic acid polyester-16-hydroxyl.

The outer surface groups of the dendrimer can have a variety of functional groups, including amidoethanol, amidoethylethanolamine, amino, hexylamide, carboxylate, succinamidyl, trimethoxysilyl, tris(hydroxymethyl)amidomethane, and 3-carbomethoxypyrrolidinone groups. In addition, these functional groups can be further treated with a coupling agent to form activated groups (as defined herein).

In one particular example, the polyamidoamine dendrimer is conjugated to a polyvalent linker molecule containing a hydrophilic polymer group: α-malemidyl-ω-N-hydroxysuccinimidyl polyethyleneglycol (MW 3400). The amino group on the surface of the polyamidoamine dendrimer is reacted with the terminal N-hydroxysuccinimidyl activated group of the linker molecule. The derivatized dendrimer is then purified, filtered, and dissolved in saline. Next, the terminal malemidyl group of the derivatized dendrimer is reacted with a sulfhydryl group of the targeting polypeptide. If the polypeptide does not contain a sulfhydryl group, then the amino group present in the polypeptide can be reacted with N-succinimidyl-5-acetylthioacetate or N-succinimidyl-5-acetylthiopropionate to introduce a protected sulfhydryl group. Alternatively, the polypeptide can be synthesized to include an additional cysteine group. The agent is associated with the derivatized dendrimer by incubating the agent and the derivatized dendrimer in a solvent and vortexing the mixture. Further examples of these approaches are described in Ke et al., J. Pharm. Sci. 97:2208-2216, 2008; Huang et al., J. Gene Med. 11:754-763, 2009; Huang et al., Biomaterials 29:238-246, 2008; and Liu et al. Biomaterials 30:4195-4202, 2009.

In another particular example, the polyamidoamine dendrimer is conjugated to a polyvalent linker molecule containing an aliphatic group: 4-sulfosuccinimidyl-6-methyl-α-(2-pyridyldithio)toluamido]hexanoate. The amino group on the surface of the polyamidoamine dendrimer is reacted with the terminal sulfosuccinimidyl activated group of the linker molecule. The derivatized dendrimer is then purified and dissolved in saline. Next, the terminal pyridyldithio group of the derivatized dendrimer is reacted with a sulfhydryl group of the polypeptide. The agent is associated with the derivatized dendrimer by incubating the agent and the derivatized dendrimer in a solvent and vortexing the mixture. Further examples of these approaches are described in Kang et al., Pharm. Res. 22:2099-2106, 2005.

Agents can be associated with the derivatized dendrimer by any number of methods, such as by covalent and non-covalent associations (e.g., ionic interaction, entrapment or physical encapsulation, hydrogen bonding, absorption, adsorption, van der Waals forces, or any combinations thereof).

Nanoparticles

Nanoparticles may be used as a transport vector in the invention. As used herein, a “nanoparticle” is a colloidal, polymeric, or elemental particle ranging in size from about 1 nm to about 1000 nm. Nanoparticles can be made up of silica, carbohydrate, lipid, or polymer molecules. Molecules can be either embedded in the nanoparticle matrix or may be adsorbed onto its surface. In one example, the nanoparticle may be made up of a biodegradable polymer such as poly(butylcyanoacrylate) (PBCA). Examples of elemental nanoparticles include carbon nanoparticles and iron oxide nanoparticles, which can then be coated with oleic acid (OA)-Pluronic. In this approach, a drug (e.g., a hydrophobic or water insoluble drug) is loaded into the nanoparticle, as described in Jain et al., Mol. Pharm. 2:194-205, 2005. Other nanoparticles are made of silica, and include those described, for example, in Burns et al., Nano Lett. 9:442-448, 2009.

Nanoparticles can be formed from any useful polymer. Examples of polymers include biodegradable polymers, such as poly(butyl cyanoacrylate), poly(lactide), poly(glycolide), poly-ε-caprolactone, poly(butylene succinate), poly(ethylene succinate), and poly(p-dioxanone); poly(ethyleneglycol); poly-2-hydroxyethylmethacrylate (poly(HEMA)); copolymers, such as poly(lactide-co-glycolide), poly(lactide)-poly(ethyleneglycol), poly(poly(ethyleneglycol)cyanoacrylate-co-hexadecylcyanoacrylate, and poly[HEMA-co-methacrylic acid]; proteins, such as fibrinogen, collagen, gelatin, and elastin; and polysaccharides, such as amylopectin, α-amylose, and chitosan.

Polymeric nanoparticles can be produced by any useful process. Using the solvent evaporation method, the polymer and agent is dissolved in a solvent to form a nanoemulsion and the solvent is evaporated. Appropriate solvent systems and surfactants can be used to obtain either oil-in-water or water-in-oil nanoemulsions. This method can optionally include filtration, centrifugation, sonication, or lyophilization. Using the nanoprecipitation method, a solution of the polymer and an agent is formed in a first solvent. Then, the solution is added to a second solvent that is miscible with the first solvent but does not solubilize the polymer. During phase separation, nanoparticles are formed spontaneously. Using the emulsion polymerization method, the monomer is dispersed into an aqueous solution to form micelles. Initiator radicals (e.g. hydroxyl ions) in the aqueous solution initiate anionic polymerization of the monomers. In another variation of the emulsion polymerization method, the agent acts as the initiator radical that promotes anionic polymerization. For example, an agent that is a photosensitizer can initiate polymerization of cyanoacrylate monomers. Additional methods include dialysis, ionic gelation, interfacial polymerization, and solvent casting with porogens.

In an example of the solvent evaporation method, the polymer is a cyanoacrylate copolymer containing a hydrophilic polymer group: poly(aminopoly(ethyleneglycol) cyanoacrylate-co-hexadecyl cyanoacrylate), which was synthesized as described in Stella et al., J. Pharm. Sci. 89:1452-1464, 2000. The polymer and agent are added to an organic solvent, where the mixture is emulsified by adding an aqueous solution. Then, the organic solvent was evaporated under reduced pressure and the resultant nanoparticles were washed and lyophilized. In the particular example of the agent being transferrin, the terminal hydroxyl group on the carbohydrate moiety of transferrin is treated with sodium periodate to form an aldehyde group and oxidized transferrin is added to the nanoparticles. Further examples of this approach are described in Li et al., Int. J. Pharm. 259:93-101, 2003; and Yu et al., Int. J. Pharm. 288:361-368, 2005.

In an example of the emulsion polymerization method, the monomer is added drowise to an acidic aqueous solution. The mixture is stirred to promote polymerization and then neutralized. The nanoparticles are then filtered, centrifuged, sonicated, and washed. In one particular example of this method, the monomer of butyl cyanoacrylate monomer is provided and the aqueous solution also includes dextran in a dilute aqueous solution of hydrochloric acid. To introduce the agent, the poly(butyl cyanoacrylate) nanoparticles are lyophilized and then resuspended in saline. Agents are added to the saline solution with the nanoparticles under constant stirring. Alternatively, the agent is added to during the polymerization process. The nanoparticles are optionally coated with a surfactant, such as polysorbate 80. Further examples of this approach are described in Kreuter et al., Brain Res. 674:171-174, 1995; Kreuter et al., Pharm. Res. 20:409-416, 2003; and Steiniger et al., Int. J. Cancer 109:759-767, 2004.

Other nanoparticles include solid lipid nanparticles (SLN). SLN approaches are described, for example, in Kreuter, Ch. 24, In V. P. Torchilin (ed), Nanoparticles as Drug Carriers pp. 527-548, Imperial College Press, 2006). Examples of lipid molecules for solid lipid nanoparticles include stearic acid and modified stearic acid, such as stearic acid-PEG 2000; soybean lechitin; and emulsifying wax. Solid lipid nanoparticles can optionally include other components, including surfactants, such as Epicuron® 200, poloxamer 188 (Pluronic® F68), Brij 72, Brij 78, polysorbate 80 (Tween 80); and salts, such as taurocholate sodium. Agents can be introduced into solid lipid nanoparticles by a number of methods discussed for liposomes and further includes high-pressure homogenization, and dispersion of microemulsions.

In one example, SLNs include stearic acid, Epicuron 2000 (surfactant), and taurocholate sodium loaded with an agent (e.g., an anticancer agent such as doxorubicin, tobramycin, idarubicin, or paclitaxel, or a paclitaxel derivative). In another example, SLNs include stearic acid, soybean lecithin, and poloxamer 188. SLNs can also be made from polyoxyl 2-stearyl ether (Brij 72), or a mixture of emulsifying wax and polyoxyl 20-stearyl ether (Brij 78) (see, e.g., Koziara et al., Pharm. Res. 20:1772-1778, 2003). In one example of making solid lipid nanoparticles, a microemulsion was formed by adding a surfactant (e.g. Brij 78 or Tween 80) to a mixture of emulsifying wax in water at 50° C. to 55° C. Emulsifying wax is a waxy solid that is prepared from cetostearyl alcohol and contains a polyoxyethylene derivative of a fatty acid ester of sorbitan. Nanoparticles are formed by cooling the mixture while stirring. The agent can be introduced by adding the agent to the heated mixture containing the emulsifying wax in water. Further examples of this approach are described in Koziara et al., Pharm. Res. 20: 1772-1778, 2003.

Nanoparticles can also include nanometer-sized micelles. Micelles can be formed from any polymers described herein. Exemplary polymers for forming micelles include block copolymers, such as poly(ethylene glycol) and poly(ε-caprolactone). In one particular example, PEO-b-PCL block copolymer is synthesized via controlled ring-opening polymerization of ε-caprolactone by using an α-methoxy-to-hydroxy-poly(ethylene glycol) as a macroinitiator. To form micelles, the PEO-b-PCL block copolymers were dissolved in an organic solvent (e.g., tetrahydrofuran) and then deionized water was added to form a micellar solution. The organic solvent was evaporated to obtain nanometer-sized micelles.

In certain embodiments, the properties of the nanoparticle are altered by coating with a surfactant. Any biocompatible surfactant may be used, for example, polysorbate surfactants, such as polysorbate 20, 40, 60, and 80 (Tween 80); Epicuron® 200; poloxamer surfactants, such as 188 (Pluronic® F68) poloxamer 908 and 1508; and Brij surfactants, such as Brij 72 and Brij 78. In other embodiments, the surfactant is covalently attached to the nanoparticle, as is described in PCT Publication No. WO 2008/085556. Such an approach may reduce toxicity by preventing the surfactant from leeching out of the nanoparticle. Nanoparticles can be optionally coated with a surfactant.

Nanoparticles can optionally be modified to include hydrophilic polymer groups (e.g., poly(ethyleneglycol) or poly(propyleneglycol)). The surface of the nanoparticle can be modified by covalently attaching hydrophilic polymer groups. Alternatively, nanoparticles can be formed by using polymers that contain hydrophilic polymer groups, such as poly[methoxy poly (ethyleneglycol) cyanoacrylate-co-hexadecyl cyanoacrylate]. Nanoparticles can be optionally cross-linked, which can be particularly use for protein-based nanoparticles.

Agents can be introduced to nanoparticles by any useful method. Agents can be incorporated into the nanoparticle at, during, or after the formation of the nanoparticle. In one example, the agent is added to the solvent with the polymer or monomer before the formation of the nanoparticles. In another example, the agent is incorporated into pre-formed nanoparticles by adsorption. In yet another example, the agent is covalently bound to the nanoparticle. The agent can be physically adsorbed to the surface of the nanoparticle with the optional step of further coating the nanoparticle with a surfactant. Examples of surfactants include polysorbate 80 (Tween 80). Further examples of this approach are described in Kreuter, Nanoparticular Carriers for Drug Delivery to the Brain, Chapter 24, in Torchilin (ed.), Nanoparticulates as Drug Carriers (2006), Imperial College Press.

Carbohydrate-Based Delivery Methods

Carbohydrate-based polymers such as chitosan can be used as a transport vector e.g., in the formation of micelles or nanoparticles. As chitosan polymers can be amphiphilic, these polymers are especially useful in the delivery of hydrophobic agents (e.g., those described herein). Exemplary chitosan polymers include quaternary ammonium palmitoyl glycol chitosan, which can be synthesized as described in Qu et al., Biomacromolecules 7:3452-3459, 2006.

Hybrid Methods

Some hybrid methods combine two or more techniques and can be useful for administering the conjugates of the invention to a cell, tissue, or organ of a subject. Virosomes, for example, combine liposomes with an inactivated virus. This combination has more efficient gene transfer in respiratory epithelial cells than either viral or liposomal methods alone. Other methods involve mixing other viral vectors with cationic lipids or hybridising viruses.

Conjugation of a Polypeptide

As used herein, a “coupling agent” is an agent that can be used to activate functional groups within the targeting peptide, linker molecule, transport vector, or agent. Examples of coupling agents include 1-ethyl-3-(3-dimethylaminopropyl)carbodiimide hydrochloride (EDC), EDC in tandem with N-hydroxysulfosuccinimide, dicyclohexylcarbodiimide, diisopropylcarbodiimide, N-ethyl-3-phenylisoxazolium-3′-sulfonate, N,N′-carbonyldiimidazole, ethylchloroformate, and trifluoromethanesulfonyl chloride.

As used herein, a “linker molecule” is a molecule that contains a spacer molecule covalently attached to one or more activated groups or functional groups. Optionally, the functional group of the linker molecule can be treated with a coupling agent to form an activated group.

As used herein, “activated group” is a functional group that allows for a covalent bond to be formed between the targeting polypeptide, agent, linker molecule, and transport vector. In one example, a covalent bond is formed between the activated group of the linker molecule and the functional group of the transport vector.

Examples of activated groups and corresponding functional groups include maleimide, which reacts with a sulfhydryl group; N-hydroxysuccinimide ester, which reacts with an amino group; N-sulfosuccinimide ester, which reacts with an amino group; imido esters, which reacts with an amino group; hydrazide or hydrazine, which reacts with an aldehyde group; haloacetyl, which reacts with a sulfhydryl group; diazirine, which can be photoactivated to create a carbene intermediate that reacts with C—H bonds; aryl azide, which can be photoactivated to create a carbene intermediate that reacts with C—H bonds; isocyanate, which reacts with an hydroxyl group; and pyridyldithio, which reacts with a sulfhydryl group. Exemplary linker molecules include BS3 ([bis(sulfosuccinimidyl)suberate]), where BS3 is a homobifunctional N-hydroxysuccinimide ester that targets accessible primary amines; NHS/EDC(N-hydroxysuccinimide and N-ethyl-'(dimethylaminopropyl)carbodimide, where NHS/EDC allows for the conjugation of primary amine groups with carboxyl groups); sulfo-EMCS ([N-e-maleimidocaproic acid]hydrazide, where sulfo-EMCS are heterobifunctional reactive groups (maleimide and NHS-ester) that are reactive toward sulfhydryl and amino groups; hydrazides, where most proteins contain exposed carbohydrates and hydrazide is a useful reagent for linking carboxyl groups to primary amines; and SATA (N-succinimidyl-5-acetylthioacetate, where SATA is reactive towards amines and adds protected sulfhydryl groups.

As used herein, a “polypeptide-transport vector conjugate” is a molecule that is capable of forming a transport vector and that is covalently bound or non-covalently bound to the targeting peptide. Examples of non-covalent bonds include ionic interaction, entrapment or physical encapsulation, hydrogen bonding, absorption, adsorption, van der Waals forces, and any combinations thereof.

Any of the molecules forming a transport vector, such as lipids (e.g., phospholipids, fatty acids, glycolipids, ceramides, glycerides, and cholesterols), carbohydrates (e.g., chitosan or chitosan derivatives), or other polymers can be conjugated to any of the targeting polypeptides described herein to form a polypeptide-transport vector conjugate. Synthetic reactions are known in the art for forming covalent bonds between functional groups present in targeting peptides, linker molecules, transport vectors, or agents. A targeting polypeptide described herein can be conjugated to a molecule forming a transport vector directly by chemical bonding (e.g., hydrophobic, covalent, hydrogen, or ionic bonds) or by using a linker molecule. Exemplary synthetic reactions for conjugating various targeting peptides and transport vectors are set forth in U.S. Pat. No. 5,747,641.

The spacer molecule within linker molecule can be of any suitable molecule. Examples of spacer molecules include aliphatic carbon groups (e.g., C₂-C₂₀ alkyl groups), cleavable heteroatomic carbon groups (e.g., C₂-C₂₀ alkyl groups with dithio groups), and hydrophilic polymer groups. Examples of hydrophilic polymer groups include poly(ethylene glycol) (PEG), polyvinylpyrrolidone, polyvinylmethylether, polymethyloxazoline, polyethyloxazoline, polyhydroxypropyloxazoline, polyhydroxypropylmethacrylamide, polymethacrylamide, polydimethylacrylamide, polyhydroxypropylmethacrylate, polyhydroxyethylacrylate, hydroxymethylcellulose, hydroxyethylcellulose, polyethyleneglycol, polyaspartamide, and a hydrophilic peptide sequence.

In one example, the hydrophilic polymer is PEG, such as a PEG chain having a molecular weight between 500-10,000 Da (e.g., between 1,000-5,000 Da such as 2,000 Da). Methoxy or ethoxy-capped analogues of PEG can also be used. These are commercially available in sizes ranging between 120-20,000 Da. Preparation of lipid-tether conjugates for use in liposomes is described, for example, in U.S. Pat. No. 5,395,619, hereby incorporated by reference. Other spacer molecules include polynucleotides (e.g., DNA or RNA), polysaccharides such as dextran or xanthan, cellulose derivatives (e.g., carboxymethyl cellulose), polystyrene, polyvinal alcohol, poly methylacrylic acid, and poly(NIPAM). Synthetic reaction schemes for activating PEG with coupling agents are set forth in U.S. Pat. Nos. 5,631,018, 5,527,528, and 5,395,619. Synthetic reaction schemes for linker molecules with PEG spacer molecules are set forth in U.S. Pat. Nos. 6,828,401, and 7,217,845.

PEG, for example, can be conjugated to a polypeptide of the invention by any means known in the art. In certain embodiments, the PEG molecule is derivatized with a linker, which is then reacted with the protein to form a conjugate. Suitable linkers include aldehydes, tresyl or tosyl linkers, dichlorotriazine or chlorotriazine, epoxide, carboxylates such as succinimidyl succinate, carbonates such as a p-nitrophenyl carbonate, benzotriazolyl carbonate, 2,3,5-trichlorophenyl carbonate, and PEG-succinimidyl carbonate, or reactive thiols suchas pyridildisufide, maleimide, vinylsulfone, and iodo acetamide. Conjugation can take place at amino groups (e.g., the N-terminal amino group or amino groups within the lysine side chain), or at thiol hydroxyl, or amide groups, depending on the linker used. See, e.g., Veronese et al., Drug Discov. Today 10:1451-1458, 2005.

A polypeptide-transport vector conjugate can be formed by covalently linking the targeting polypeptide to a transport vector molecule using a linker molecule. For example, the transport vector molecule forms a covalent bond with the proximal end of a bivalent linker molecule and the targeting polypeptide forms a covalent bond with the distal end of the linker molecule. In a particular example, the transport vector is a lipid molecule covalently bound to a linker molecule: 1,2-distearoyl-sn-glycero-3-phosphoethanolamine-N-[methoxy(polyethylene glycol)-2000]-maleimide. The amino group on the targeting polypeptide is modified with Traut's reagent (2-iminothiolane) to form sulfhydryl groups. The modified targeting polypeptide is then conjugated to the maleimide group of the lipid molecule to form a polypeptide-lipid conjugate.

The polypeptide may be conjugated to the transport vector through activated groups, sulfhydryl groups, amino groups (amines) and/or carbohydrates or any appropriate functional groups. Homopolyvalent and heteropolyvalent linker molecules (conjugation agents) are available from many commercial sources. Regions available for cross-linking may be found on the polypeptides of the present invention. The linker molecule may comprise a flexible arm, such as for example, a short arm (<2 carbon chain), a medium-size arm (from 2-5 carbon chain), or a long arm (3-6 carbon chain).

The linker molecule can be polyvalent or monovalent. A monovalent linker molecule has only one activated group available for forming a covalent bond. However, the monovalent linker molecule can include one or more functional groups that can be chemically modified by using a coupling agent, as described herein, to form a second activated group. For example, a terminal hydroxyl group of the linker molecule can be activated by any number of coupling agents. Examples of coupling agents include N-hydroxysuccinimide, ethylchloroformate, dicyclohexylcarbodiimide, and trifluoromethanesulfonyl chloride. See, e.g. U.S. Pat. Nos. 5,395,619 and 6,316,024.

A polyvalent linker molecule has two or more activated groups. The activated groups in the linker molecule can be the same, as in a homopolyvalent linker molecule, or different, as in a heteropolyvalent linker molecule. Heteropolyvalent linker molecules allow for conjugating a polypeptide and a transport vector with different functional groups. Examples of heteropolyvalent linker molecules include polyoxyethylene-bis(p-nitrophenyl carbonate), mal-PEG-DSPE, diisocyanate, succinimidyl 4-hydrazinonicotinate acetone hydrazone.

Examples of homopolyvalent linker molecules with two activated groups include disuccinimidyl glutarate, disuccinimidyl suberate, bis(sulfosuccinimidyl) suberate, bis(NHS)PEG₅, bis(NHS)PEG₉, dithiobis(succinimidyl propionate), 3,3′-dithiobis(sulfosuccinimidylpropionate), disuccinimidyl tartrate, bis[2-(succinimido oxycarbonyloxy)ethyl]sulfone, ethylene glycol bis[succinimidylsuccinate]), ethylene glycol bis[sulfosuccinimidylsuccinate]), dimethyl adipimidate, dimethyl pimelimidate, dimethyl suberimidate, dimethyl 3,3′-dithiobispropionimidate, 1,5-difluoro-2,4-dinitrobenzene, bis-maleimidoethane, 1,4-bismaleimidobutane, bismaleimidohexane, 1,8-bis-maleimidodiethyleneglycol, 1,11-bis-maleimido-triethyleneglycol, 1,4-di-[3′-(2′-pyridyldithio)-propionamido]butane, 1,6-hexane-bis-vinylsulfone, and bis-[b-(4-azidosalicylamido)ethyl]disulfide.

Examples of homopolyvalent linker molecules with three activated groups include tris-succinimidyl aminotriacetate, pitris(hydroxymethyl) phosphino]propionic acid, and tris[2-maleimidoethyl]amine.

Examples of heteropolyvalent linker molecules include those with an maleimide activated group and a succinimide activated group, such as N-[α-maleimidoacetoxy]succinimide ester, N-[β-maleimidopropyloxy]-succinimide ester, N-[γ-maleimidobutyryloxy]succinimide ester, m-maleimidobenzoyl-N-hydroxysuccinimide ester, succinimidyl 4-[N-male imidomethyl]cyclohexane-1-carboxylate, N-[ε-maleimidocaproyloxy]succinimide ester, and succinimidyl 4-[p-maleimidophenyl]butyrate, including N-sulfosuccinimidyl derivatives; those with a PEG spacer molecule, such as succinimidyl-([N-maleimidopropionamido]-(ethyleneglycol)ₓ)ester, wherein x is from 2 to 24; those with a pyridyldithio activated group and a succinimide activated group, such as N-succinimidyl-3-(2-pyridyldithio)propionate, succinimidyl 6-(3-[2-pyridyldithio]-propionamido)hexanoate, 4-succinimidyloxycarbonyl-methyl-a-[2-pyridyldithio]toluene, and 4-sulfosuccinimidyl-6-methyl-a-(2-pyridyldithio)toluamido]hexanoate); those with a haloacetyl activated group and a succinimide activated group, such as N-succinimidyl iodoacetate and N-succinimidyl[4-iodoacetyl]aminobenzoate; those with an aryl azide activated group and a succinimide activated group, such as N-hydroxysuccinimidyl-4-azidosalicylic acid, sulfosuccinimidyl[4-azidosalicylamido]-hexanoate, and N-succinimidyl-6-(4′-azido-2′-nitrophenylamino) hexanoate; those with an diazirine activated group and a succinimide activated group, such as succinimidyl 4,4′-azipentanoate and succinimidyl 6-(4,4′-azipentanamido)hexanoate; N-[4-(p-azidosalicylamido) butyl]-3′-(2′-pyridyldithio)propionamide; N-[β-maleimidopropionic acid]hydrazide; N-(ε-maleimidocaproic acid) hydrazide; 4-(4-N-maleimidophenyl)butyric acid hydrazide hydrochloride; (N-[κ-maleimidoundecanoic acid]-hydrazide); 3-(2-pyridyldithio)propionyl hydrazide; p-azidobenzoyl hydrazide; and N-[p-maleimidophenyl]isocyanate.

### Methods of Making Polypeptide-Transport Vector Conjugates

To form a polypeptide-transport vector conjugate of the invention, at least two general approaches can be used. In a first approach, a transport vector containing the agent (e.g., any described herein) is formed. Then, a polypeptide described herein is conjugated to the transport vector. In a second approach, the conjugation of the polypeptide to a molecule forming the transport vector (e.g., any described herein) is performed first, and then the transport vector is formed subsequently using the conjugated molecule. In either approach, the polypeptide may be conjugated through a tether molecule.

A polypeptide-transport vector conjugate can be formed in a step-wise process. For example, the transport vector molecule is first attached to the linker molecule and transport vectors are formed containing the transport vector molecule. Then, the transport vector is incubated with the targeting polypeptide to form a covalent bond with the linker molecule. In a particular example, a lipid molecule is attached to the linker molecule and the resultant compound is used to form liposomes. Then, the liposomes are incubated with a solution containing the targeting polypeptide to attach the polypeptide to the distal end of the linker molecule.

In another example, the transport vector is covalently linked to a linker molecule with an activated group, the targeting polypeptide is covalently linked to a second linker molecule, and then the modified transport vector and modified polypeptide are reacted together to form a covalent bond between the first linker molecule and a second linker molecule. For example, the amino group of a transport vector forms a covalent bond by displacing the N-hydroxysuccinimidyl group of the linker molecule succinimidyl 4-formylbenzoate. This modified target vector has a terminal carbonyl group on the linker molecule. Then, the amino group of the polypeptide forms a covalent bond by displacing the N-hydroxysuccinimidyl group of the linker molecule succinimidyl 4-hydrazinonicotinate acetone hydrazone. This modified polypeptide has a terminal hydrazine group on the linker molecule. Finally, the modified target vector and the modified polypeptide are combined to form a covalent bond between the hydrazine group of the modified polypeptide and the terminal carbonyl group of the target vector.

In another example, polyoxyethylene-(p-nitrophenyl carbonate)-phosphoethanolamine is used in the formation of lipid micelles containing siRNA molecules. Briefly, in this example, polyoxyethylene-bis (p-nitrophenyl carbonate) ((pNP)₂-PEG) is conjugated to a lipid capable of forming liposomes or micelles such as 1,2-dipalmitoyl-sn-glycero-3-phosphoethanolamine (DPPE), resulting in production of pNP-PEG-PE. This molecule can then, in turn, be conjugated to a polypeptide (e.g., any described herein) to form a peptide-PEG-PE conjugate. This conjugate can then be used in the formation of liposomes that contain PEG moieties which serve as anchors for binding polypeptide molecules on the external face of the liposome. See, e.g., Zhang et al., J Control. Release 112:229-239, 2006.

Production of lipid vectors can also be achieved by conjugating a polypeptide to a liposome following its formation. In one example of this procedure, a mixture of lipids suitable for encapsulating a molecule and having sufficient in vivo stability are provided, where some of the lipids are attached to a tether (such as PEG) containing a linker (e.g., any linker described herein). The mixture is dried, reconstituted in aqueous solution with the desired polynucleotide, and subject to conditions capable of forming liposomes (e.g., sonication or extrusion). A polypeptide described herein is then conjugated to the linker on the tether. In one particular example of this method, the mixture of 93% 1-palmitoyl-2-oleoyl-sn-glycerol-3-phosphocholine (POPC), 3% didodecyldimethylammonium bromide (DDAB), 3% distearoylphosphatidylethanolamine (DSPE)-PEG2000 and 1% DSPE-PEG2000-maleimide is provided. This mixture is then prepared in chloroform, evaporated under nitrogen, and then dissolved in Tris buffer to which the desired polynucleotide is added. The mixture is then passed through a series of polycarbonate filters of reduced pore size 400 nm to 50 nm to generate 80-100 nm liposomes. The liposomes are mixed with a nuclease to remove unencapsulated polynucleotides. If the polynucleotide is a DNA molecule, DNA endonuclease I and exonuclease III. The polypeptide described herein can then be conjugated to the DSPE-PEG200 that contains the linker (e.g., maleimide or any linker herein. These lipid vectors, which contain a polynucleotide and are conjugated to a polypeptide described herein can then be administered to a subject to deliver the polynucleotide across the BBB or to specific tissues. Further examples of this approach are described in Boado, Pharm. Res. 24:1772-1787, 2007; Pardridge, Pharm. Res. 24:1733-1744, 2007; and Zhang et al., Clin. Canc. Res. 10:3667-3677, 2004.

Alternatively, the polypeptide-transport vector conjugate is formed without the use of a linker molecule. Rather, a zero-length coupling agent is used to activate the functional groups within the transport vector or the targeting polypeptide without introducing additional atoms. Examples of zero-length coupling agents include dicyclohexylcarbodiimide and ethylchloroformate.

### Therapeutic Agents

The polypeptide-transport vector conjugates of the invention may be bound to or may contain any therapeutic agent known in the art. Exemplary agents include polynucleotides (e.g., RNAi agents and gene therapy vectors (e.g., capable of expressing therapeutic polypeptides or RNAi agents), anticancer therapeutics, polypeptides (e.g., GLP-1 agonists such as GLP-1, exendin-4, and analogs thereof; leptin; neurotensin; GDNF, BDNF, or analogs thereof), and hydrophobic agents.

Polynucleotides

The polypeptide-transport vector conjugates of the invention can be bound to or can contain any polynucleotide. Exemplary polynucleotides include expression vectors (e.g., a plasmid) and therapeutic polynucleotides (e.g., RNAi agents). Any type of polynucleotide known in the art, such as double and single-stranded DNA and RNA molecules of any length, conformation, charge, or shape (e.g., linear, concatemer, circular (e.g., a plasmid), nicked circular, coiled, supercoiled, or charged) can be used. Polynucleotides can contain 5′ and 3′ terminal modifications and include blunt and overhanging nucleotides at these termini, or combinations thereof. In certain embodiments of the invention the polynucleotide is or encodes an RNAi sequence (e.g., an siRNA, shRNA, miRNA, or dsRNA nucleotide sequence) that can silence a targeted gene product. The polynucleotide can be, for example, a DNA molecule, an RNA molecule, or a modified form thereof.

Expression Vectors

In certain embodiments, the polynucleotide contains a sequence that is capable of expressing a protein. The polynucleotide may encode a polypeptide (e.g., a therapeutic polypeptide) or may encode a therapeutic polynucleotide (e.g., an RNAi agent such as those described herein). Any expression system known in the art may be used and any suitable disease may be treated using a expression system (e.g., a plasmid) known in the art. For example, a plasmid encoding a cytokine (e.g., interferon α) can be provided to a subject having a cancer (Horton et al, Proc. Natl. Acad. Sci. USA 96:1553-1558, 1999). Other approaches are described, for example, in Mahvi et al. (Cancer Gene Ther. 14:717-723, 2007). Here, a plasmid expressing IL-12 was injected into metastatic tumors, resulting in decreased tumor size. Diseases such as cardiovascular disorders can also be treated similarly, e.g., using growth factors such as FGF-2. In one example, such growth factors are administered to a subject suffering from myocardial ischemia using a plasmid vector encoding the growth factor. Transport of plasmid DNA to tissues such as liver may also be desirable for treating or vaccinating against cancers such as hepatoma or other liver cancer. See, e.g., Chou et al. (Cancer Gene Ther. 13:746-752, 2006).

In treatment of diseases that are caused by a defect or deficiency in a gene or protein (e.g., lysosomal storage disorders), it may be desirable for the expression vector to encode the defective or deficient polypeptide. For example, treatment of a lysosomal storage disease may be accomplished by using an polynucleotide that is capable of expressing the deficient protein, as shown in Table 2.

Other approaches include using a DNA plasmid that encodes an RNAi agent, such as an shRNA nucleotide sequence (e.g., EGFR). Upon localization to a target cell, the RNAi molecule is transcribed from the plasmid and causes down-regulation of a target gene product.

In another embodiment, the polypeptide-transport vectors of the invention include a viral polynucleotide or virus particles (e.g., adenovirus, retrovirus) which carries a viral genome including a recombinant polynucleotide sequence (e.g., coding for an RNAi agent or a therapeutic polypeptide). Upon transport to the target cells or through the BBB, the viral polynucleotide or particles bind and transduce target cells. The viral genome is then expressed in the target cell, which results in expression of the recombinant sequence.

RNA Interference Agents

The polypeptide-transport vectors of the invention may be bound to or may contain an RNAi agent. Exemplary RNAi agents include siRNA, shRNA, dsRNA, and miRNA agents.

In certain embodiments, the RNAi agent is a small interfering RNA (siRNA). These are are short (usually 21 nt) and are usually double-stranded RNA (dsRNA). siRNA molecules may have, for example, 1 or 2 nucleotide overhangs on the 3′ ends, or may be blunt-ended. Each strand has a 5′ phosphate group and a 3′ hydroxyl group. Most siRNA molecules are 18 to 23 nucleotides in length, however a skilled practitioner may vary this sequence length (e.g., to increase or decrease the overall level of gene silencing). Almost any gene for which the sequence is known can thus be targeted based on sequence complementarity with an appropriately tailored siRNA. See, for example, Zamore et al., Cell 101:25-33, 2000; Bass, Nature 411:428-429, 2001; Elbashir et al., Nature 411:494-498, 2001; and PCT Publication Nos. WO 00/44895, WO 01/36646, WO 99/32619, WO 00/01846, WO 01/29058, WO 99/07409, and WO 00/44914. Methods for preparing an siRNA molecule are known in the art and described in, for example, U.S. Pat. No. 7,078,196.

A short hairpin RNA (shRNA) molecule may also be used in the invention. shRNA are single-stranded RNA molecules in which a tight hairpin loop structure is present, allowing complementary nucleotides within the same strand to form bonds. shRNA can exhibit reduced sensitivity to nuclease degradation as compared to siRNA. Once inside a target cell, shRNA are processed and effect gene silencing by the same mechanism described above for siRNA.

Double-stranded RNA (dsRNA) can also be used in the invention. Any double-stranded RNA that can be cleaved in cell into siRNA molecules that target a specific mRNA can be used. Methods of preparing dsRNA for use as RNAi agents are described in, for example, U.S. Pat. No. 7,056,704.

MicroRNAs (miRNA) can also be used in the invention. miRNA are single-stranded RNA molecules that can silence a target gene using the same or similar mechanisms as siRNA and shRNA agents. miRNA molecules of 21 to 23 nucleotides in length are often used, as these are generally the most effective for gene silencing; however, a skilled practitioner may vary the sequence length as desired.

Any of the RNAi molecules described herein may be modified or substituted with nucleotide analogs, e.g., as described herein.

RNAi agents may be capable of silencing any gene where a reduction in expression of that gene is therapeutically beneficial. Examples of RNAi targets include growth factors (e.g., epidermal growth factor (EGF), vascular endothelial growth factor (VEGF), transforming growth factor-beta (TGF-β)), growth factor receptors, including receptor tyrosine kinases (e.g., EGF receptor (EGFR), including Her2/neu (ErbB), VEGF receptor (VEGFR), platelet-derived growth factor receptor (PDGFR), cytokines, chemokines, kinases, including cytoplasmic tyrosine and serine/threonine kinases (e.g., focal adhesion kinase, cyclin-dependent kinase, SRC kinases, syk-ZAP70 kinases, BTK kinases, RAF kinase, MAP kinases (including ERK), and Wnt kinases), phosphatases, regulatory GTPases (e.g., Ras protein), transcription factors (e.g., MYC), hormones and hormone receptors (e.g., estrogen and estrogen receptor), anti-apoptotic molecules (e.g., survivin, Bcl-2, Bcl-xL), oncogenes (e.g., tumor suppressor regulators such as mdm2), enzymes (e.g., superoxide dismutase 1 (SOD-1), α, β (BACE), and γ secretases), and other proteins (e.g., Huntingtin (Htt protein), amyloid precursor protein (APP), sorting nexins (including SNX6), α-synuclein, LINGO-1, Nogo-A, and Nogo receptor 1 (NgR-1)), and glial fibrillary acidic protein. Table 2 illustrates the relationship between exemplary RNAi targets and diseases and is not meant to limit the scope of the present invention.

Exemplary RNAi sequences capable of silencing EGFR are GGAGCUGCCCAUGAGAAAU (SEQ ID NO:117) and AUUUCUCAUGGGCAGCUCC (SEQ ID NO:118). VEGF can be silenced by an RNAi molecule having the sequence GGAGTACCCTGATGAGATC (SEQ ID NO:119). Exemplary RNAi sequences to silence α-synuclein include AAGGACCAGTTGGGCAAGAAT (SEQ ID NO:120), AACAGTGGCTGAGAAGACCAA (SEQ ID NO:121), AAAAAGGACCAGTTGGGCAAG (SEQ ID NO:122), AAAAGGACCAGTTGGGCAAGA (SEQ ID NO:123), AAAGGACCAGTTGGGCAAGAA (SEQ ID NO:124), AAGATATGCCTGTGGATCCTG (SEQ ID NO:125), AAATGCCTTCTGAGGAAGGGT (SEQ ID NO:126), AATGCCTTCTGAGGAAGGGTA (SEQ ID NO:127), and AAGACTACGAACCTGAAGCCT (SEQ ID NO:128); see, e.g., U.S. Patent Application Publication No. 2007/0172462. Exemplary RNAi sequences to silence β-secretase (β-amyloid cleavage enzyme 1 (BACE-1)) include AAGACTGTGGCTACAACATTC (SEQ ID NO:129); see, e.g., U.S. Patent Application Publication No. 2004/0220132. Additional RNAi sequences for use in the agents of the invention may be either commercially available (e.g., from Dharmacon or Ambion) or the practitioner may use one of several publicly available software tools for the construction of viable RNAi sequences (e.g., The siRNA Selection Server, maintained by MIT/Whitehead; available at: http://jura.wi.mitedu/bioc/siRNAext/). Examples of diseases or conditions, and targets to which RNAi agents can be directed that may be useful in treatment of such diseases, are shown in Table 2.

Modified Nucleic Acids

Modified nucleic acids, including modified DNA or RNA molecules, may be used in the in place of naturally occurring nucleic acids in the polynucleotides described herein. Modified nucleic acids can improve the half-life, stability, specificity, delivery, solubility, and nuclease resistance of the polynucleotides described herein. For example, siRNA agents can be partially or completed composed of nucleotide analogs that confer the beneficial qualities described above. As described in Elmén et al. (Nucleic Acids Res. 33:439-447, 2005), synthetic, RNA-like nucleotide analogs (e.g., locked nucleic acids (LNA)) can be used to construct siRNA molecules that exhibit silencing activity against a target gene product.

Modified nucleic acids include molecules in which one or more of the components of the nucleic acid, namely sugars, bases, and phosphate moieties, are different from that which occurs in nature, preferably different from that which occurs in the human body. Nucleoside surrogates are molecules in which the ribophosphate backbone is replaced with a non-ribophosphate construct that allows the bases to the presented in the correct spatial relationship such that hybridization is substantially similar to what is seen with a ribophosphate backbone, e.g., non-charged mimics of the ribophosphate backbone.

Modifications can be incorporated into any double-stranded RNA (e.g., any RNAi agent (e.g., siRNA, shRNA, dsRNA, or miRNA), RNA-like, DNA, and DNA-like molecules. It may be desirable to modify one or both of the antisense and sense strands of a polynucleotide. As polynucleotides are polymers of subunits or monomers, many of the modifications described below occur at a position which is repeated within a nucleic acid, e.g., a modification of a base, or a phosphate moiety, or the non-linking O of a phosphate moiety. In some cases the modification will occur at all of the subject positions in the nucleic acid but in many, and in fact in most, cases it will not. For example, a modification may only occur at a 3′ or 5′ terminal position, may only occur in a terminal region, e.g., at a position on a terminal nucleotide or in the last 2, 3, 4, 5, or 10 nucleotides of a strand. A modification may occur in a double strand region, a single strand region, or in both. For example, a phosphorothioate modification at a non-linking O position may only occur at one or both termini, may only occur in terminal regions, e.g., at a position on a terminal nucleotide or in the last 2, 3, 4, 5, or 10 nucleotides of a strand, or may occur in double strand and single strand regions, particularly at termini. Similarly, a modification may occur on the sense strand, antisense strand, or both. In some cases, the sense and antisense strand will have the same modifications or the same class of modifications, but in other cases the sense and antisense strand will have different modifications, e.g., in some cases it may be desirable to modify only one strand, e.g., the sense strand.

Two prime objectives for the introduction of modifications into the polynucleotides described herein is their increased protection from degradation in biological environments and the improvement of pharmacological properties, e.g., pharmacodynamic properties, which are discussed further below. Other suitable modifications to a sugar, base, or backbone of a polynucleotide are described in PCT Publication No. WO 2004/064737, hereby incorporated by reference. A polynucleotide can include a non-naturally occurring base, such as the bases described in PCT Publication No. WO 2004/094345, hereby incorporated by reference. A polynucleotide can include a non-naturally occurring sugar, such as a non-carbohydrate cyclic carrier molecule. Exemplary features of non-naturally occurring sugars for use in the polynucleotides described herein are described in PCT Publication No. WO 2004/094595, hereby incorporated by reference.

Any of the polynucleotides described herein can include an internucleotide linkage (e.g., the chiral phosphorothioate linkage) useful for increasing nuclease resistance. In addition, or in the alternative, a polynucleotide can include a ribose mimic for increased nuclease resistance. Exemplary internucleotide linkages and ribose mimics for increased nuclease resistance are described in U.S. Patent Application Publication No. 2005/0164235.

Any polynucleotide described herein can include ligand-conjugated monomer subunits and monomers for oligonucleotide synthesis. Exemplary monomers are described in U.S. Patent Application Publication No. 2005/0107325.

Any polynucleotide can have a ZXY structure, such as is described in U.S. Patent Application Publication No. 2005/0164235.

Any polynucleotide can be complexed with an amphipathic moiety. Exemplary amphipathic moieties for use with RNAi agents are described in U.S. Patent Application Publication No. 2005/0164235.

Anticancer Agents

Any anticancer agent may be used in the compositions and methods of the invention. Exemplary anticancer agents include alkylating agents (e.g., busulfan, dacarbazine, ifosfamide, hexamethylmelamine, thiotepa, dacarbazine, lomustine, cyclophosphamide chlorambucil, procarbazine, altretamine, estramustine phosphate, mechlorethamine, streptozocin, temozolomide, and Semustine), platinum agents (e.g., spiroplatin, tetraplatin, ormaplatin, iproplatin, ZD-0473 (AnorMED), oxaliplatin, carboplatin, lobaplatin (Aeterna), satraplatin (Johnson Matthey), BBR-3464 (Hoffmann-La Roche), SM-11355 (Sumitomo), AP-5280 (Access), and cisplatin), antimetabolites (e.g., azacytidine, floxuridine, 2-chlorodeoxyadenosine, 6-mercaptopurine, 6-thioguanine, cytarabine, 2-fluorodeoxy cytidine, methotrexate, tomudex, fludarabine, raltitrexed, trimetrexate, deoxycoformycin, pentostatin, hydroxyurea, decitabine (SuperGen), clofarabine (Bioenvision), irofulven (MGI Pharma), DMDC (Hoffmann-La Roche), ethynylcytidine (Taiho), gemcitabine, and capecitabine), topoisomerase inhibitors (e.g., amsacrine, epirubicin, etoposide, teniposide or mitoxantrone, 7-ethyl-10-hydroxy-camptothecin, dexrazoxanet (TopoTarget), pixantrone (Novuspharma), rebeccamycin analogue (Exelixis), BBR-3576 (Novuspharma), rubitecan (SuperGen), irinotecan (CPT-11), topotecan, exatecan mesylate (Daiichi), quinamed (ChemGenex), gimatecan (Sigma-Tau), diflomotecan (Beaufour-Ipsen), TAS-103 (Taiho), elsamitrucin (Spectrum), J-107088 (Merck & Co), BNP-1350 (BioNumerik), CKD-602 (Chong Kun Dang), KW-2170 (Kyowa Hakko), and hydroxycamptothecin (SN-38)), antitumor antibiotics (e.g., valrubicin, therarubicin, idarubicin, rubidazone, plicamycin, porfiromycin, mitoxantrone (novantrone), amonafide, azonafide, anthrapyrazole, oxantrazole, losoxantrone, MEN-10755 (Menarini), GPX-100 (Gem Pharmaceuticals), epirubicin, mitoxantrone, and doxorubicin), antimitotic agents (e.g., colchicine, vinblastine, vindesine, dolastatin 10 (NCI), rhizoxin (Fujisawa), mivobulin (Warner-Lambert), cemadotin (BASF), RPR 109881A (Aventis), TXD 258 (Aventis), epothilone B (Novartis), T 900607 (Tularik), T 138067 (Tularik), cryptophycin 52 (Eli Lilly), vinflunine (Fabre), auristatin PE (Teikoku Hormone), BMS 247550 (BMS), BMS184476 (BMS), BMS 188797 (BMS), taxoprexin (Protarga), SB 408075 (GlaxoSmithKline), vinorelbine, trichostatin A, E7010 (Abbott), PG-TXL (Cell Therapeutics), IDN 5109 (Bayer), A 105972 (Abbott), A 204197 (Abbott), LU 223651 (BASF), D 24851 (ASTAMedica), ER-86526 (Eisai), combretastatin A4 (BMS), isohomohalichondrin-B (PharmaMar), ZD 6126 (AstraZeneca), AZ10992 (Asahi), IDN-5109 (Indena), AVLB (Prescient NeuroPharma), azaepothilone B (BMS), BNP-7787 (BioNumerik), CA-4 prodrug (OXiGENE), dolastatin-10 (NIH), CA-4 (OXiGENE), docetaxel, vincristine, and paclitaxel), aromatase inhibitors (e.g., aminoglutethimide, atamestane (BioMedicines), letrozole, anastrazole, YM-511 (Yamanouchi), formestane, and exemestane), thymidylate synthase inhibitors (e.g., pemetrexed (Eli Lilly), ZD-9331 (BTG), nolatrexed (Eximias), and CoFactor™ (BioKeys)), DNA antagonists (e.g., trabectedin (PharmaMar), glufosfamide (Baxter International), albumin+³²P (Isotope Solutions), thymectacin (NewBiotics), edotreotide (Novartis), mafosfamide (Baxter International), apaziquone (Spectrum Pharmaceuticals), and O⁶-benzylguanine (Paligent)), Farnesyltransferase inhibitors (e.g., arglabin (NuOncology Labs), lonafarnib (Schering-Plough), BAY-43-9006 (Bayer), tipifarnib (Johnson & Johnson), and perillyl alcohol (DOR BioPharma)), pump inhibitors (e.g., CBT-1 (CBA Pharma), tariquidar (Xenova), MS-209 (Schering AG), zosuquidar trihydrochloride (Eli Lilly), biricodar dicitrate (Vertex)), histone acetyltransferase inhibitors (e.g., tacedinaline (Pfizer), SAHA (Aton Pharma), MS-275 (Schering AG), pivaloyloxymethyl butyrate (Titan), depsipeptide (Fujisawa)), metalloproteinase inhibitors (e.g., Neovastat (Aeterna Laboratories), marimastat (British Biotech), CMT-3 (CollaGenex), BMS-275291 (Celltech)), Ribonucleoside reductase inhibitors (e.g., gallium maltolate (Titan), triapine (Vion), tezacitabine (Aventis), didox (Molecules for Health)), TNFα agonists/antagonists (e.g., virulizin (Lorus Therapeutics), CDC-394 (Celgene), and revimid (Celgene)), Endothelin A receptor antagonists (e.g., atrasentan (Abbott), ZD-4054 (AstraZeneca), and YM-598 (Yamanouchi)), Retinoic acid receptor agonists (e.g., fenretinide (Johnson & Johnson), LGD-1550 (Ligand), and alitretinoin (Ligand)), Immuno-modulators (e.g., interferon, oncophage (Antigenics), GMK (Progenies), adenocarcinoma vaccine (Biomira), CTP-37 (AVI BioPharma), IRX-2 (Immuno-Rx), PEP-005 (Peplin Biotech), synchrovax vaccines (CTL Immuno), melanoma vaccine (CTL Immuno), p21 RAS vaccine (GemVax), dexosome therapy (Anosys), pentrix (Australian Cancer Technology), ISF-154 (Tragen), cancer vaccine (Intercell), norelin (Biostar), BLP-25 (Biomira), MGV (Progenics), β-alethine (Dovetail), and CLL therapy (Vasogen)), hormonal and antihormonal agents (e.g., estrogens, conjugated estrogens, ethinyl estradiol, chlortrianisen, idenestrol, hydroxyprogesterone caproate, medroxyprogesterone, testosterone, testosterone propionate; fluoxymesterone, methyltestosterone, diethylstilbestrol, megestrol, bicalutamide, flutamide, nilutamide, dexamethasone, prednisone, methylprednisolone, prednisolone, aminoglutethimide, leuprolide, octreotide, mitotane, P-04 (Novogen), 2-methoxyestradiol (EntreMed), arzoxifene (Eli Lilly), tamoxifen, toremofine, goserelin, Leuporelin, and bicalutamide), photodynamic agents (e.g., talaporfin (Light Sciences), Theralux (Theratechnologies), motexafin gadolinium (Pharmacyclics), Pd-bacteriopheophorbide (Yeda), lutetium texaphyrin (Pharmacyclics), and hypericin), and kinase inhibitors (e.g., imatinib (Novartis), leflunomide (Sugen/Pharrnacia), ZD1839 (AstraZeneca), erlotinib (Oncogene Science), canertinib (Pfizer), squalamine (Genaera), SU5416 (Pharmacia), SU6668 (Pharmacia), ZD4190 (AstraZeneca), ZD6474 (AstraZeneca), vatalanib (Novartis), PKI166 (Novartis), GW2016 (GlaxoSmithKline), EKB-509 (Wyeth), trastuzumab (Genentech), OSI-774 (Tarceva™), CI-1033 (Pfizer), SU11248 (Pharmacia), RH3 (York Medical), genistein, radicinol, EKB-569 (Wyeth), kahalide F (PharmaMar), CEP-701 (Cephalon), CEP-751 (Cephalon), MLN518 (Millenium), PKC412 (Novartis), phenoxodiol (Novogen), C225 (ImClone), rhu-Mab (Genentech), MDX-H210 (Medarex), 2C4 (Genentech), MDX-447 (Medarex), ABX-EGF (Abgenix), IMC-1C11 (ImClone), tyrphostins, gefitinib (Iressa), PTK787 (Novartis), EMD 72000 (Merck), Emodin, and Radicinol).

Other anticancer agents include SR-27897 (CCK A inhibitor, Sanofi-Synthelabo), tocladesine (cyclic AMP agonist, Ribapharm), alvocidib (CDK inhibitor, Aventis), CV-247 (COX-2 inhibitor, Ivy Medical), P54 (COX-2 inhibitor, Phytopharm), CapCell™ (CYP450 stimulant, Bavarian Nordic), GCS-100 (gal3 antagonist, GlycoGenesys), G17DT immunogen (gastrin inhibitor, Aphton), efaproxiral (oxygenator, Allos Therapeutics), PI-88 (heparanase inhibitor, Progen), tesmilifene (histamine antagonist, YM BioSciences), histamine (histamine H2 receptor agonist, Maxim), tiazofurin (IMPDH inhibitor, Ribapharm), cilengitide (integrin antagonist, Merck KGaA), SR-31747 (IL-1 antagonist, Sanofi-Synthelabo), CCI-779 (mTOR kinase inhibitor, Wyeth), exisulind (PDE V inhibitor, Cell Pathways), CP-461 (PDE V inhibitor, Cell Pathways), AG-2037 (GART inhibitor, Pfizer), WX-UK1 (plasminogen activator inhibitor, Wilex), PBI-1402 (PMN stimulant, ProMetie LifeSciences), bortezomib (proteasome inhibitor, Millennium), SRL-172 (T cell stimulant, SR Pharma), TLK-286 (glutathione S transferase inhibitor, Telik), PT-100 (growth factor agonist, Point Therapeutics), midostaurin (PKC inhibitor, Novartis), bryostatin-1 (PKC stimulant, GPC Biotech), CDA-II (apoptosis promotor, Everlife), SDX-101 (apoptosis promotor, Salmedix), rituximab (CD20 antibody, Genentech, carmustine, mitoxantrone, bleomycin, absinthin, chrysophanic acid, cesium oxides, ceflatonin (apoptosis promotor, ChemGenex), BCX-1777 (PNP inhibitor, BioCryst), ranpirnase (ribonuclease stimulant, Alfacell), galarubicin (RNA synthesis inhibitor, Dong-A), tirapazamine (reducing agent, SRI International), N-acetylcysteine (reducing agent, Zambon), R-flurbiprofen (NF-kappaB inhibitor, Encore), 3CPA (NF-kappaB inhibitor, Active Biotech), seocalcitol (vitamin D receptor agonist, Leo), 131-I-TM-601 (DNA antagonist, TransMolecular), eflornithine (ODC inhibitor, ILEX Oncology), minodronic acid (osteoclast inhibitor, Yamanouchi), indisulam (p53 stimulant, Eisai), aplidine (PPT inhibitor, PharmaMar), gemtuzumab (CD33 antibody, Wyeth Ayerst), PG2 (hematopoiesis enhancer, Pharmagenesis), Immunol™ (triclosan oral rinse, Endo), triacetyluridine (uridine prodrug, Wellstat), SN-4071 (sarcoma agent, Signature BioScience), TransMID-107™ (immunotoxin, KS Biomedix), PCK-3145 (apoptosis promotor, Procyon), doranidazole (apoptosis promotor, Pola), CHS-828 (cytotoxic agent, Leo), trans-retinoic acid (differentiator, NIH), MX6 (apoptosis promotor, MAXIA), apomine (apoptosis promotor, ILEX Oncology), urocidin (apoptosis promotor, Bioniche), Ro-31-7453 (apoptosis promotor, La Roche), brostallicin (apoptosis promotor, Pharmacia), β-lapachone, gelonin, cafestol, kahweol, caffeic acid, and Tyrphostin AG. The invention may also use analogs of any of these agents (e.g., analogs having anticancer activity).

Paclitaxel and Related Compounds

In particular embodiments, the anticancer agent is paclitaxel or a paclitaxel analog. Paclitaxel has the formula:

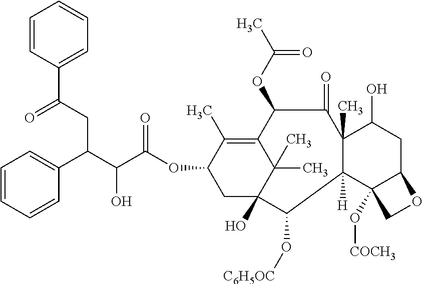

Structural analogs of paclitaxel are described in U.S. Pat. No. 6,911,549, and can be described by the formula:

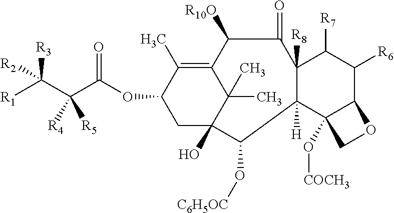

where R₁ is selected from the group consisting of —CH₃; —C₆H₅, or phenyl substituted with 1, 2 or 3 C₁-C₄ alkyl, C₁-C₃ alkoxy, halo, C₁-C₃ alkylthio, trifluoromethyl, C₂-C₆ dialkylamino, hydroxyl, or nitro; and 2-furyl, 2-thienyl, 1-naphthyl, 2-naphthyl or 3,4-methylenedioxyphenyl; R₂ is selected from the group consisting of —H, —NHC(O)H, —NHC(O)C₁-C₁₀ alkyl (preferably —NHC(O)C₄-C₆ alkyl), —NHC(O)phenyl, —NHC(O)phenyl substituted with one, 2, or 3 C₁-C₄ alkyl, C₁-C₃ alkoxy, halo, C₁-C₃ alkylthio, trifluoromethyl, C₂-C₆ dialkylamino, hydroxy or nitro, —NHC(O)C(CH₃)═CHCH₃, —NHC(O)OC(CH₃)₃, —NHC(O)OCH₂ phenyl, —NH₂, —NHSO₂-4-methylphenyl, —NHC(O)(CH₂)₃COOH, —NHC(O)-4-(SO₃H)phenyl, —OH, —NHC(O)-1-adamantyl, —NHC(O)O-3-tetrahydrofuranyl, —NHC(O)O-4-tetrahydropyranyl, —NHC(O)CH₂C(CH₃)₃, —NHC(O)C(CH₃)₃, —NHC(O)OC₁-C₁₀ alkyl, —NHC(O)NHC₁-C₁₀ alkyl, —NHC(O)NHPh, —NHC(O)NHPh substituted with one, 2, or 3 C₁-C₄ alkyl, C₁-C₃ alkoxy, halo, C₁-C₃ alkylthio, trifluoromethyl, C₂-C₆ dialkylamino, or nitro, —NHC(O)C₃-C₈ cycloalkyl, —NHC(O)C(CH₂CH₃)₂CH₃, —NHC(O)C(CH₃)₂CH₂Cl, —NHC(O)C(CH₃)₂CH₂CH₃, phthalimido, —NHC(O)-1-phenyl-1-cyclopentyl, —NHC(O)-1-methyl-1-cyclohexyl, —NHC(S)NHC(CH₃)₃, —NHC(O)NHCC(CH₃)₃, or —NHC(O)NHPh; R₃ is selected from the group consisting of —H, —NHC(O)phenyl, or —NHC(O)OC(CH₃)₃, with the overall proviso that one of R₂ and R₃ is —H but R₂ and R₃ are not both —H; R₄ is —H or selected from the group consisting of —OH, —OAc (—OC(O)CH₃), —OC(O)OCH₂C(Cl)₃, —OCOCH₂CH₂NH₃⁺HCOO⁻, —NHC(O)phenyl, —NHC(O)OC(CH₃)₃, —OCOCH₂CH₂COOH and pharmaceutically acceptable salts thereof, —OCO(CH₂)₃COOH and pharmaceutically acceptable salts thereof, and —OC(O)—Z—C(O)—R′ [where Z is ethylene (—CH₂CH₂), propylene (—CH₂CH₂CH₂—), —CH═CH—, 1,2-cyclohexane, or 1,2-phenylene, R′ is —OH, —OH base, —NR′₂R′₃, —OR′₃, —SR′₃, —OCH₂C(O)NR′₄R′₅ where R′₂ is —H or —CH₃, R′₃ is —(CH₂)ₙNR′₆R′₇ or (CH₂)ₙN⁺R′₆R′₇R′₈X⁻ where n is 1-3, R′₄ is —H or —C₁-C₄ alkyl, R′₅ is —H, —C₁-C₄ alkyl, benzyl, hydroxyethyl, —CH₂CO₂H, or dimethylaminoethyl, R′₆ and R′₇ are —CH₃, —CH₂CH₃, benzyl or R′₆ and R′₇ together with the nitrogen of NR′₆R′₇ form a pyrrolidino, piperidino, morpholino, or N-methylpiperizino group; R′₈ is —CH₃, —CH₂CH₃ or benzyl, X⁻ is halide, and base is NH₃, (HOC₂H₄)₃N, N(CH₃)₃, CH₃N(C₂H₄)₂NH, NH₂(CH₂)₆NH₂, N-methylglucamine, NaOH, or KOH], —OC(O)(CH₂)ₙNR²R³ [where n is 1-3, R² is —H or —C₁-C₃ alkyl and R³ is —H or —C₁-C₃ alkyl], —OC(O)CH(R″)NH₂ [where R″ is selected from the group consisting of —H, —CH₃, —CH₂CH(CH₃)₂, —CH(CH₃)CH₂CH₃, —CH(CH₃)₂, —CH₂ phenyl, —(CH₂)₄NH₂, —CH₂CH₂COOH, —(CH₂)₃NHC(═NH)NH₂], the residue of the amino acid proline, —OC(O)CH═CH₂, —C(O)CH₂CH₂C(O)NHCH₂CH₂SO₃⁻Y⁺, —OC(O)CH₂CH₂C(O)NHCH₂CH₂CH₂SO₃⁻Y⁺ wherein Y⁺ is Na⁺ or N⁺ (Bu)₄, —OC(O)CH₂CH₂C(O)OCH₂CH₂OH; R₅ is —H or —OH, with the overall proviso that when R₅ is —OH, R₄ is —H and with the further proviso that when R₅ is —H, R₄ is not —H; R₆ is —H:—H when R₇ is α-R₇₁:β-R₇₂ where one of R₇₁, and R₇₂ is —H and the other of R₇₁ and R₇₂ is —X where X is halo and R₈ is —CH₃; R₆ is —H:—H when R₇ is α-H:β-R₇₄ where R₇₄ and R₈ are taken together to form a cyclopropyl ring; R₁₀ is —H or —C(O)CH₃; and pharmaceutically acceptable salts thereof when the compound contains either an acidic or basic functional group.

Particular paclitaxel analogs include ((azidophenyl)ureido)taxoid, (2α,5α,7β,9α,10β,13α)-5,10,13,20-tetraacetoxytax-11-ene-2,7,9-triol, (2α,5α,9α,10β)-2,9,10-triacetoxy-5-((β-D-glucopyranosyl)oxy)-3,11-cyclotax-11-en-13-one, 1β-hydroxybaccatin I,1,7-dihydroxytaxinine, 1-acety-5,7,10-deacetyl-baccatin I, 1-dehydroxybaccatin VI, 1-hydroxy-2-deacetoxy-5-decinnamoyl-taxinine j, 1-hydroxy-7,9-dideacetylbaccatin I,1-hydroxybaccatin I,10-acetyl-4-deacetyltaxotere, 10-deacetoxypaclitaxel, 10-deacetyl baccatin III dimethyl sulfoxide disolvate, 10-deacetyl-10-(3-aminobenzoyl)paclitaxel, 10-deacetyl-10-(7-(diethylamino)coumarin-3-carbonyl)paclitaxel, 10-deacetyl-9-dihydrotaxol, 10-deacetylbaccatine III, 10-deacetylpaclitaxel, 10-deacetyltaxinine, 10-deacetyltaxol, 10-deoxy-10-C-morpholinoethyl docetaxel, 10-O-acetyl-2-O-(cyclohexylcarbonyl)-2-debenzoyltaxotere, 10-O-sec-aminoethyl docetaxel, 11-desmethyllaulimade, 13-deoxo-13-acetyloxy-7,9-diacetyl-1,2-dideoxytaxine, 13-deoxybaccatin III, 14-hydroxy-10-deacetyl-2-O-debenzoylbacatin III, 14-hydroxy-10-deacetylbaccatin III, 14β-benzoyloxy-13-deacetylbaccatin IV, 14β-benzoyloxy-2-deacetylbaccatin VI, 14β-benzoyloxybaccatin IV, 19-hydroxybaccatin III, 2′,2″-methylenedocetaxel, 2′,2″-methylenepaclitaxel, 2′-(valyl-leucyl-lysyl-PABC)paclitaxel, 2′-acetyltaxol, 2′-O-acetyl-7-O—(N-(4′-fluoresceincarbonyl)alanyl)taxol, 2,10,13-triacetoxy-taxa-4(20),11-diene-5,7,9-triol, 2,20-O-diacetyltaxumairol N, 2-(4-azidobenzoyl)taxol, 2-deacetoxytaxinine J, 2-debenzoyl-2-m-methoxybenozyl-7-triethylsilyl-13-oxo-14-hydroxybaccatin III 1,14-carbonate, 2-O-(cyclohexylcarbonyl)-2-debenzoylbaccatin III 13—O-(N-(cyclohexylcarbonyl)-3-cyclohexylisoserinate), 2α,7β,9α,10β,13α-pentaacetoxyltaxa-4 (20), 11-dien-5-ol, 2α,5α,7β,9α,13α-pentahydroxy-10β-acetoxytaxa-4(20),11-diene, 2α,7β,9α,10β,13-pentaacetoxy-11β-hydroxy-5α-(3′-N,N-dimethylamino-3′-phenyl)-propionyloxytaxa-4(20),12-diene, 2α,7β-diacetoxy-5α,10β,13β-trihydroxy-2(3-20)abeotaxa-4(20),11-dien-9-one, 2α,9α-dihydroxy-10β,13α-diacetoxy-5α-(3′-methylamino-3′-phenyl)-propionyloxytaxa-4(20),11-diene, 2α-hydroxy-7β,9α,10β,13α-tetraacetoxy-5α-(2′-hydroxy-3′-N,N-dimethylamino-3′-phenyl)-propionyloxytaxa-4(20),11-diene, 3′-(4-azidobenzamido)taxol, 3′-N-(4-benzoyldihydrocinnamoyl)-3′-N-debenzoylpaclitaxel, 3′-N-m-aminobenzamido-3′-debenzamidopaclitaxel, 3′-p-hydroxypaclitaxel, 3,11-cyclotaxinine N,N-2,4-deacetyltaxol, 5,13-diacetoxy-taxa-4(20),11-diene-9,10-diol, 5-O-benzoylated taxinine K, 5-O-phenylpropionyloxytaxinine A, 5α,13α-diacetoxy-10β-cinnamoyloxy-4(20),11-taxadien-9α-ol, 6,3′-p-dihydroxypaclitaxel, 6-α-hydroxy-7-deoxy-10-deacetylbaccatin-III, 6-fluoro-10-acetyldocetaxel, 6-hydroxytaxol, 7,13-diacetoxy-5-cinnamyloxy-2(3-20)-abeo-taxa-4(20),11-diene-2,10-diol, 7,9-dideacetylbaccatin VI, 7-(5′-biotinylamidopropanoyl)paclitaxel, 7-acetyltaxol, 7-deoxy-10-deacetylbaccatin-III, 7-deoxy-9-dihydropaclitaxel, 7-epipaclitaxel, 7-methylthiomethylpaclitaxel, benzoyldihydrocinnamoyl)paclitaxel, 7-O—(N-(4′-fluoresceincarbonyl)alanyl)taxol, 7-xylosyl-10-deacetyltaxol, 8,9-single-epoxy brevifolin, 9-dihydrobaccatin III, 9-dihydrotaxol, 9α-hydroxy-2α,10β,13α-triacetoxy-5α-(3′-N,N-dimethyl amino-3′-phenyl)-propionyloxytaxa-4(20),11-diene, baccatin III, baccatin III 13-O-(N-benzoyl-3-cyclohexylisoserinate), BAY59, benzoyltaxol, BMS181339, BMS185660, BMS188797, brevifoliol, butitaxel, cephalomannine, dantaxusin A, dantaxusin B, dantaxusin C, dantaxusin D, dibromo-10-deacetylcephalomannine, DJ927, docetaxel, Flutax 2, glutarylpaclitaxel 6-aminohexanol glucuronide, IDN 5109, IDN 5111, IDN 5127, IDN 5390, isolaulimalide, laulimalide, MST 997, N-(paclitaxel-2′-O-(2-amino)phenylpropionate)-O-(β-glucuronyl)carbarnate, N-(paclitaxel-2′-O-3,3-dimethyl butanoate)-O-(β-glucuronyl)carbamate, N-debenzoyl-N-(3-(dimethylamino)benzoyl)paclitaxel, nonataxel, octreotide-conjugated paclitaxel, paclitaxel-transferrin, PNU 166945, poly(ethylene glycol)-conjugated paclitaxel-2′-glycinate, polyglutamic acid-paclitaxel, protax, protaxel, RPR 109881A, SB T-101187, SB T-1102, SB T-1213, SB T-1214, SB T-1250, SB T-12843, tasumatrol E, tasumatrol F, tasumatrol G, taxa-4(20), 11(12)-dien-5-yl acetate, taxa-4(20),11(12)-diene-5-ol, taxane, taxchinin N, taxcultine, taxezopidine M, taxezopidine N, taxine, taxinine, taxinine A, taxinine M, taxinine NN-1, taxinine N,N-7, taxol C-7-xylose, taxol-sialyl conjugate, taxumairol A, taxumairol B, taxumairol G, taxumairol H, taxumairol I, taxumairol K, taxumairol M, taxumairol N, taxumairol O, taxumairol U, taxumairol V, taxumairol W, taxumairol-X, taxumairol-Y, taxumairol-Z, taxusin, taxuspinanane A, taxuspinanane B, taxuspine C, taxuspine D, taxuspine F, taxuyunnanine C, taxuyunnanine S, taxuyunnanine T, taxuyunnanine U, taxuyunnanine V, tRA-96023, and wallifoliol. Other paclitaxel analogs include 1-deoxypaclitaxel, 10-deacetoxy-7-deoxypaclitaxel, 10-O-deacetylpaclitaxel 10-monosuccinyl ester, 10-succinyl paclitaxel, 12b-acetyloxy-2a,3,4,4a,5,6,9,10,11,12,12a,12b-dodecahydro-4,11-dihydroxy-12-(2,5-dimethoxybenzyloxy)-4-a,8,13,13-tetramethyl-5-oxo-7,11-methano-1H-cyclodeca(3,4)benz(1,2-b)oxet-9-yl 3-(tert-butyloxycarbonyl)amino-2-hydroxy-5-methyl-4-hexaenoate, 130-nm albumin-bound paclitaxel, 2′-paclitaxel methyl 2-glucopyranosyl succinate, 3′-(4-azidophenyl)-3′-dephenylpaclitaxel, 4-fluoropaclitaxel, 6,6,8-trimethyl-4,4-a,5,6,7,7a,8,9-octahydrocyclopenta(4,5)cyclohepta(1,2-c)-furan-4,8-diol 4-(N-acetyl-3-phenylisoserinate), 6,6,8-trimethyl-4,4-a,5,6,7,7a,8,9-octahydrocyclopenta(4,5)cyclohepta(1,2-c)-furan-4,8-diol 4-(N-tert-butoxycarbonyl-3-phenylisoserinate), 7-(3-methyl-3-nitrosothiobutyryl)paclitaxel, 7-deoxypaclitaxel, 7-succinylpaclitaxel, A-Z-CINN 310, AI-850, albumin-bound paclitaxel, AZ 10992,isotaxel, MAC321, MBT-0206, NK105, Pacliex, paclitaxel poliglumex, paclitaxel-EC-1 conjugate, polilactofate, and TXD 258. Other paclitaxel analogs are described in U.S. Pat. Nos. 4,814,470; 4,857,653; 4,942,184; 4,924,011; 4,924,012; 4,960,790; 5,015,744; 5,157,049; 5,059,699; 5,136,060; 4,876,399; and 5,227,400.

Etoposide and Related Compounds

Etoposide or a related compound may also be used in the compositions and methods of the invention. In some embodiments, the compounds is a podophyllotoxin derivative having a structure according to the formula:

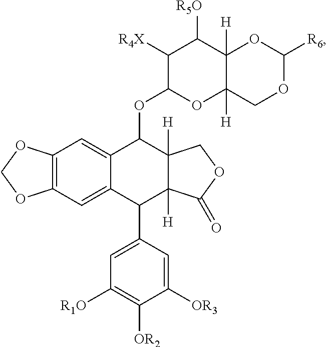

or a stereoisomer thereof, where each R₁, R₂, and R₃ is selected, independently, from H, optionally substituted C₁₋₆ alkyl, C(O)R₈, P(O)(OR₉)(OR₁₀), S(O)₂(OR₉), or a hydrolyzable linker Y that comprises a covalent bond to an amino acid of the polypeptide; X is O or NR₇; each R₄, R₅, and R₇ is selected, independently, from H, optionally substituted C₁₋₆ alkyl, C(O)R₈, or a hydrolyzable linker Y that comprises a covalent bond to an amino acid of the polypeptide; R₆ is H, optionally substituted C₁₋₆ alkyl, optionally substituted aryl, optionally substituted heteroaryl; R₈ is selected from optionally substituted C₁₋₆ alkyl or optionally substituted aryl; each R₉ and R₁₀ is selected, independently, from H, optionally substituted C₁₋₆ alkyl, or optionally substituted aryl; and n is 1, 2, 3, 4, 5, 6, 7, or 8. In certain embodiments, the etoposide derivative is conjugated at the 2′ or 3′ hydroxyl group. Further examples of such conjugation strategies are described in U.S. Provisional Application Nos. 61/105,654, filed Oct. 15, 2008, and 61/171,010, filed Apr. 20, 2009.

Other analogs of etoposide include etoposide phosphate (ETOPOPHOS®), where the phenolic —OH is replaced with —OP(O)(OH)₂, or any pharmaceutically acceptable salt thereof (e.g., —OP(O)(ONa)₂). Etoposide phosphate has improved water solubility compared to etoposide.

Other etoposide analogs include those where the phenolic —OH is replaced with an acyloxy group (e.g., —OC(O)R₈, as described herein) such as the following compound:

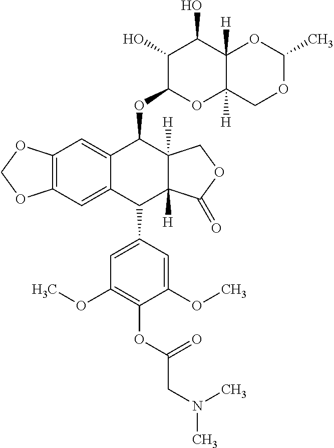

(“etoposide 4′-dimethylglycine” or “etoposide_{DMG}”). These acylated etoposide analogs can also show improved water solubility relative to etoposide when covalently attached to any of the polypeptides described herein.

Other exemplary podophyllotoxin analogs include teniposide and NK611.

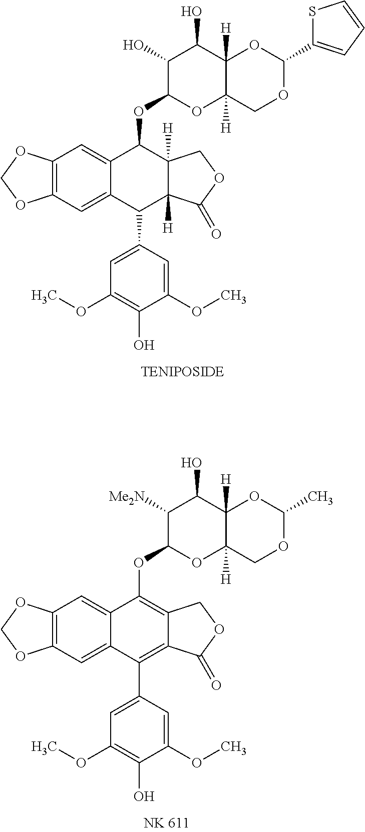

Still other podophyllotoxin analogs suitable for use in the invention are described in U.S. Pat. Nos. 4,567,253; 4,609,644; 4,900,814; 4,958,010; 5,489,698; 5,536,847; 5,571,914; 6,051,721; 6,107,284; 6,475,486; 6,610,299; 6,878,746; 6,894,075; 7,087,641; 7,176,236; 7,241,595; 7,342,114; and 7,378,419; and in U.S. Patent Publication Nos. 2003/0064482, 2003/0162722, 2004/0044058, 2006/0148728, and 2007/0249651, each of which is hereby incorporated by reference.

Doxorubicin and Related Compounds

In some embodiments, the anti-cancer agent is doxorubicin (hydroxydaunorubicin or Adriamycin®) or a related compound such as epirubicin (Ellence® or Pharmorubicin®). The structures of these exemplary compounds are shown below. Doxorubicin and doxorubicin analogs can be covalently attached to an amino acid in any of the polypeptides described herein through a hydrolyzable covalent linker bonded to, for example, the 14-hydroxyl group.

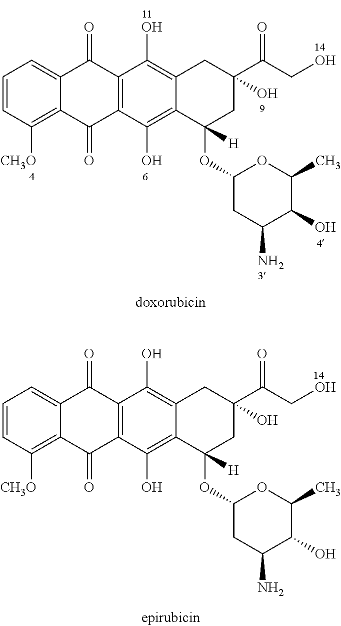

Doxorubicin analogs can be described generally by the following formula:

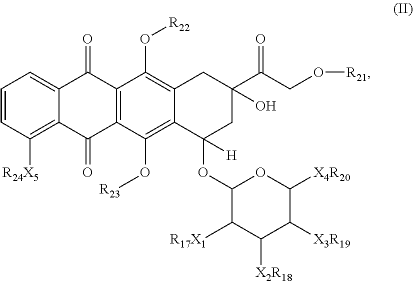

where each X₁, X₂, X₃, X₄, and X₅ is selected, independently, from a covalent bond, O, or NR₂₅; each R₁₇, R₁₈, R₁₉, R₂₀, R₂₀, R₂₁, R₂₂, R₂₃, R₂₄, and R₂₅, is selected, independently, from H, optionally substituted C₁₋₆ alkyl, optionally substituted C₂₋₆ alkenyl, optionally substituted C₂₋₆ alkynyl, optionally substituted cycloalkyl, optionally substituted heterocyclyl, or is a hydrolyzable linker Y as defined herein.

When a compound of Formula (II) is attached to any of the polypeptides described herein, one of R₁₇, R₁₈, R₁₉, R₂₀, R₂₀, R₂₁, R₂₂, R₂₃, R₂₄, and R₂₅ is Y. In certain embodiments, R₂₁ is Y.

Other doxorubicin analogs are described in U.S. Pat. Nos. 4,098,884, 4,301,277, 4,314,054, 4,464,529, 4,585,859, 4,672,057, 4,684,629, 4,826,964, 5,200,513, 5,304,687, 5,594,158, 5,625,043, and 5,874,412, each of which is hereby incorporated by reference.

Polypeptides

The compositions and methods of the present invention may include any polypeptide having biological activity (e.g., polypeptide therapeutics) known in the art. Exemplary polypeptides are described, for example, in U.S. Provisional Application No. 61/200,947, filed Dec. 5, 2008, which is hereby incorporated by reference.

GLP-1 Agonists

The therapeutic agent used in the invention may be any GLP-1 agonist known in the art. Particular GLP-1 agonists include GLP-1, exendin-4, and analogs thereof. Exemplary analogs are described below.

Exendin-4 and Exendin-4 Analogs.

Exendin-4 and exendin-4 analogs can also be used in the compositions and methods of the invention. The compounds of the invention can include fragments of the exendin-4 sequence. Exendin-4 has the sequence.

His-Gly-Glu-Gly-Thr-Phe-Thr-Ser-Asp-Leu-Ser-Lys-Gln-Met-Glu-Glu-Glu-Ala-Val-Arg-
Leu-Phe-Ile-Glu-Trp-Leu-Lys-Asn-Gly-Gly-Pro-Ser-Ser-Gly-Ala-Pro-Pro-Pro-Ser-NH₂

Particular exendin-4 analogs include those having a cysteine substitution (e.g., [Cys³²]exendin-4) or a lysine substitution (e.g., [Lys³⁹]exendin-4).

Exendin analogs are also described in U.S. Pat. No. 7,157,555 and include those of the formula:

X₁-X₂-X₃-Gly-Thr-X₄-X₅-X₆-X₇-X₈-Ser-Lys-Gln-X₉-Glu-Glu-Glu-Ala-Val-Arg-Leu-X₁₀-
X₁₁-X₁₂-X₁₃-Leu-Lys-Asn-Gly-Gly-X₁₄-Ser-Ser-Gly-Ala-X₁₅-X₁₆-X₁₇-X₁₈-Z

where X₁ is His, Arg or Tyr; X₂ is Ser, Gly, Ala or Thr; X₃ is Asp or Glu; X₄ is Phe, Tyr or Nal; X₅ is Thr or Ser; X₆ is Ser or Thr; X₇ is Asp or Glu; X₈ is Leu, Ile, Val, pGly or Met; X₉ is Leu, Ile, pGly, Val or Met; X₁₀ is Phe, Tyr, or Nal; X₁₁ is Ile, Val, Leu, pGly, t-BuG or Met; X₁₂ is Glu or Asp; X₁₃ is Trp, Phe, Tyr, or Nal; X₁₄, X₁₅, X₁₆ and X₁₇ are independently Pro, HPro, 3Hyp, 4Hyp, TPro, N-alkylglycine, N-alkyl-pGly or N-alkylalanine; X₁₈ is Ser, Thr, or Tyr; and Z is —OH or —NH₂ (e.g., with the proviso that the compound is not exendin-3 or exendin-4.)

Preferred N-alkyl groups for N-alkylglycine, N-alkyl-pGly and N-alkylalanine include lower alkyl groups (e.g., C₁₋₆ alkyl or C₁₋₄ alkyl).

In certain embodiments, X₁ is H is or Tyr (e.g., H is). X₂ can be Gly. X₉ can be Leu, pGly, or Met. X₁₃ can be Trp or Phe. X₄ can be Phe or Nal; X₁₁ can be Ile or Val, and X₁₄, X₁₅, X₁₆ and X₁₇ can be independently selected from Pro, HPro, TPro, or N-alkylalanine (e.g., where N-alkylalanine has a N-alkyl group of 1 to about 6 carbon atoms). In one aspect, X₁₅, X₁₆, and X₁₇ are the same amino acid residue. X₁₈ may be Ser or Tyr (e.g., Ser). Z can be —NH₂.

In other embodiments, X₁ is H is or Tyr (e.g., H is); X₂ is Gly; X₄ is Phe or Nal; X₉ is Leu, pGly, or Met; X₁₀ is Phe or Nal; X₁₁ is Ile or Val; X₁₄, X₁₅, X₁₆, and X₁₇ are independently selected from Pro, HPro, TPro, or N-alkylalanine; and Xᵢₛ is Ser or Tyr, (e.g., Ser). Z can be —NH₂.

In other embodiments, X₁ is H is or Arg; X₂ is Gly; X₃ is Asp or Glu; X₄ is Phe or napthylalanine; X₅ is Thr or Ser; X₆ is Ser or Thr; X₇ is Asp or Glu; X₈ is Leu or pGly; X₉ is Leu or pGly; X₁₀ is Phe or Nal; X₁₁ is Ile, Val, or t-butyltylglycine; X₁₂ is Glu or Asp; X₁₃ is Trp or Phe; X₁₄, X₁₅, X₁₆, and X₁₇ are independently Pro, HPro, TPro, or N-methylalanine; X₁₈ is Ser or Tyr: and Z is —OH or —NH₂ (e.g., where the compound is not exendin-3 or exendin-4). Z can be —NH₂.

In another embodiment, X₉ is Leu, Ile, Val, or pGly (e.g., Leu or pGly) and X₁₃ is Phe, Tyr, or Nal (e.g., Phe or Nal). These compounds can exhibit advantageous duration of action and be less subject to oxidative degradation, both in vitro and in vivo, as well as during synthesis of the compound.

Other exendin analogs are described in U.S. Pat. Nos. 7,157,555 and 7,223,725, and include compounds of the formula:

X₁-X₂-X₃-Gly-X₅-X₆-X₇-X₈-X₉-X₁₀-X₁₁-X₁₂-X₁₃-X₁₄-X₁₅-X₁₆-X₁₇-Ala-X₁₉-X₂₀-X₂₁-X₂₂-
X₂₃-X₂₄-X₂₅-X₂₆-X₂₇-X₂₈-Z₁

where X₁ is His, Arg, or Tyr; X₂ is Ser, Gly, Ala, or Thr; X₃ is Asp or Glu; X₅ is Ala or Thr; X₆ is Ala, Phe, Tyr, or Nal; X₇ is Thr or Ser; X₈ is Ala, Ser, or Thr; X₉ is Asp or Glu; X₁₀ is Ala, Leu, Ile, Val, pGly, or Met; X₁₁ is Ala or Ser; X₁₂ is Ala or Lys; X₁₃ is Ala or Gln; X₁₄ is Ala, Leu, Ile, pGly, Val, or Met; X₁₅ is Ala or Glu; X₁₆ is Ala or Glu; X₁₇ is Ala or Glu; X₁₉ is Ala or Val; X₂₀ is Ala or Arg; X₂₁ is Ala or Leu; X₂₂ is Phe, Tyr, or Nal; X₂₃ is Ile, Val, Leu, pGly, t-BuG, or Met; X₂₄ is Ala, Glu, or Asp; X₂₅ is Ala, Trp, Phe, Tyr, or Nal; X₂₆ is Ala or Leu; X₂₇ is Ala or Lys; X₂₈ is Ala or Asn; Z₁ is —OH, —NH₂, Gly-Z₂, Gly-Gly-Z₂, Gly-Gly-X₃₁—Z₂, Gly-Gly-X₃₁-Ser-Z₂, Gly-Gly-X₃₁-Ser-Ser-Z₂, Gly-Gly-X₃₁-Ser-Ser-Gly-Z₂, Gly-Gly-X₃₁-Ser-Ser-Gly-Ala-Z₂, Gly-Gly-X₃₁-Ser-Ser-Gly-Ala-X₃₆—Z₂, Gly-Gly-X₃₁-Ser-Ser-Gly-Ala-X₃₆—X₃₇—Z₂ or Gly-Gly-X₃₁-Ser-Ser-Gly-Ala-X₃₆—X₃₇—X₃₈—Z₂; X₃₁, X₃₆, X₃₇, and X₃₈ are independently Pro, HPro, 3Hyp, 4Hyp, TPro, N-alkylglycine, N-alkyl-pGly or N-alkylalanine; and Z₂ is —OH or —NH₂ (e.g., provided that no more than three of X₅, X₆, X₈, X₁₀, X₁₁, X₁₂, X₁₃, X₁₄, X₁₅, X₁₆, X₁₇, X₁₉, X₂₀, X₂₁, X₂₄, X₂₅, X₂₆, X₂₇, and X₂₈ are Ala). Preferred N-alkyl groups for N-alkylglycine, N-alkyl-pGly, and N-alkylalanine include lower alkyl groups of 1 to about 6 carbon atoms (e.g., 1 to 4 carbon atoms).

In certain embodiments, X₁ is H is or Tyr (e.g., H is). X₂ can be Gly. X₁₄ can be Leu, pGly, or Met. X₂₅ can be Trp or Phe. In some embodiments, X₆ is Phe or Nal, X₂₂ is Phe or Nal, and X₂₃ is Ile or Val. X₃₁, X₃₆, X₃₇, and X₃₈ can be independently selected from Pro, HPro, TPro, and N-alkylalanine. In certain embodiments, Z₁ is —NH₂ or Z₂ is —NH₂.

In another embodiment, X₁ is H is or Tyr (e.g., H is); X₂ is Gly; X₆ is Phe or Nal; X₁₄ is Leu, pGly, or Met; X₂₂ is Phe or Nal; X₂₃ is Ile or Val; X₃₁, X₃₆, X₃₇, and X₃₈ are independently selected from Pro, HPro, TPro, and N-alkylalanine. In particular embodiments, Z₁ is —NH₂.

In another embodiment, X₁ is H is or Arg; X₂ is Gly or Ala; X₃ is Asp or Glu; X₅ is Ala or Thr; X₆ is Ala, Phe, or naphthylalanine; X₇ is Thr or Ser; X₈ is Ala, Ser, or Thr; X₉ is Asp or Glu; X₁₀ is Ala, Leu, or pGly; X₁₁ is Ala or Ser; X₁₂ is Ala or Lys; X₁₃ is Ala or Gln; X₁₄ is Ala, Leu, or pGly; X₁₅ is Ala or Glu; X₁₆ is Ala or Glu; X₁₇ is Ala or Glu; X₁₉ is Ala or Val; X₂₀ is Ala or Arg; X₂₁ is Ala or Leu; X₂₂ is Phe or Nal; X₂₃ is Ile, Val or t-BuG; X₂₄ is Ala, Glu or Asp; X₂₅ is Ala, Trp or Phe; X₂₆ is Ala or Leu; X₂₇ is Ala or Lys; X₂₈ is Ala or Asn; Z₁ is —OH, —NH₂, Gly-Z₂, Gly-Gly-Z₂, Gly-Gly-X₃₁—Z₂, Gly-Gly X₃₁-Ser-Z₂, Gly-Gly-X₃₁ Ser-Ser-Z₂, Gly-Gly-X₃₁ Ser-Ser-Gly-Z₂, Gly-Gly-X₃₁ Ser-Ser-Gly Ala-Z₂, Gly-Gly-X₃₁ Ser-Ser-Gly-Ala-X₃₆—Z₂, Gly-Gly-X₃₁-Ser-Ser-Gly-Ala-X₃₆—X₃₇—Z₂, Gly-Gly-X₃₁-Ser-Ser-Gly-Ala-X₃₆—X₃₇—X₃₈—Z₂; X₃₁, X₃₆, X₃₇ and X₃₈ being independently Pro HPro, TPro or N-methylalanine; and Z₂ being —OH or —NH₂ (e.g., provided that no more than three of X₃, X₅, X₆, X₈, X₁₀, X₁₁, X₁₂, X₁₃, X₁₄, X₁₅, X₁₆, X₁₇, X₁₉, X₂₀, X₂₁, X₂₄, X₂₅, X₂₆, X₂₇ and X₂₈ are Ala).

In yet another embodiment, X₁₄ is Leu, Ile, Val, or pGly (e.g., Leu or pGly), and X₂₅ is Phe, Tyr, or Nal (e.g., Phe or Nal).

Exendin analogs described in U.S. Pat. No. 7,220,721 include compounds of the formula:

X₁-X₂-X₃-X₄-X-₅-X₆-X₇-X₈-X₉-X₁₀-X₁₁-X₁₂-X₁₃-X₁₄-X₁₅-X₁₆-X₁₇-Ala-X₁₉-X₂₀-X₂₁-X₂₂-X₂₃-
X₂₄-X₂₅-X₂₆-X₂₇-X₂₈-Z₁

where X₁ is His, Arg, Tyr, Ala, Norval, Val, or Norleu; X₂ is Ser, Gly, Ala, or Thr; X₃ is Ala, Asp, or Glu; X₄ is Ala, Norval, Val, Norleu, or Gly; X₅ is Ala or Thr; X₆ is Phe, Tyr, or Nal; X₇ is Thr or Ser; X₈ is Ala, Ser or Thr; X₉ is Ala, Norval, Val, Norleu, Asp, or Glu; X₁₀ is Ala, Leu, Ile, Val, pGly, or Met; Xᵢᵢ is Ala or Ser; X₁₂ is Ala or Lys; X₁₃ is Ala or Gln; X₁₄ is Ala, Leu, Ile, pGly, Val, or Met; X₁₅ is Ala or Glu; X₁₆ is Ala or Glu; X₁₇ is Ala or Glu; X₁₉ is Ala or Val; X₂₀ is Ala or Arg; X₂₁ is Ala or Leu; X₂₂ is Phe, Tyr, or Nal; X₂₃ is Ile, Val, Leu, pGly, t-BuG, or Met; X₂₄ is Ala, Glu, or Asp; X₂₅ is Ala, Tip, Phe, Tyr, or Nal; X₂₆ is Ala or Leu; X₂₇ is Ala or Lys; X₂₈ is Ala or Asn; Z₁ is —OH, —NH₂, Gly-Z₂, Gly-Gly-Z₂, Gly-Gly-X₃₁—Z₂, Gly-Gly-X₃₁-Ser-Z₂, Gly-Gly-X₃₁-Ser-Ser-Z₂, Gly-Gly-X₃₁-Ser-Ser-Gly-Z₂, Gly-Gly-X₃₁ Ser-Ser-Gly-Ala-Z₂, Gly-Gly-X₃₁-Ser-Ser-Gly-Ala-X₁₃—Z₂, Gly-Gly-X₃₁-Ser-Ser-Gly-Ala-X₃₆—X₃₇—Z₂, Gly-Gly-X₃₁-Ser-Ser-Gly-Ala-X₃₆—X₃₇—X₃₁—Z₂, or Gly-Gly-X₃₁-Ser-Ser-Gly-Ala-X₃₆—X₃₇—X₃₈—X₃₉—Z₂; where X₃₁, X₃₆, X₃₇, and X₃₈ are independently Pro, HPro, 3Hyp, 4Hyp, TPro, N-alkylglycine, N-alkyl-pGly, or N-alkylalanine; and Z₂ is —OH or —NH₂ (e.g., provided that no more than three of X₃, X₄, Xₛ, X₈, X₉, X₁₀, X₁₁, X₁₂, X₁₃, X₁₄, X₁₅, X₁₆, X₁₇, X₁₉, X₂₀, X₂₁, X₂₄, X₂₅, X₂₆, X₂₇, and X₂₈ are Ala and/or provided also that, if X₁ is His, Arg, or Tyr, then at least one of X₃, X₄, and X₉ is Ala).

Particular examples of exendin-4 analogs include exendin-4(1-30), exendin-4(1-30) amide, exendin-4(1-28) amide, [Leu¹⁴,Phe²⁵]exendin-4 amide, [Leu¹⁴,Phe²⁵]exendin-4(1-28) amide, and [Leu¹⁴,Ala²²,Phe²⁵]exendin-4(1-28) amide.

U.S. Pat. No. 7,329,646 describes exendin-4 analogs having the general formula:

His-Gly-Glu-Gly-Thr-Phe-Thr-Ser-Asp-Leu-Ser-Lys-Gln-X₁₄-Glu-Glu-Glu-Ala-
Val-X₂₀-Leu-Phe-Ile-Glu-Trp-Leu-Lys-Asn-Gly-Gly-Pro-Ser-Ser-Gly-Ala-Pro-Pro-Pro-
Ser-X₄₀.

where X₁₄ is Arg, Leu, Ile, or Met; X₂₀ is His, Arg, or Lys; X₄₀ is Arg-OH, —OH, —NH₂, or Lys-OH. In certain embodiments, when X₁₄ is Met and X₂₀ is Arg, X₄₀ cannot be —NH₂. Other exendin-4 derivatives include [(Ile/Leu/Met)¹⁴,(His/Lys)²⁰,Arg⁴⁰]exendin-4; [(not Lys/not Arg)¹²,(not Lys/not Arg)²⁰,(not Lys/not Arg)²⁷,Arg⁴⁰]exendin-4; and [(not Lys/not Arg)²⁰,Arg⁴⁰]exendin-4. Particular exendin-4 analogs include [Lys²⁰,Arg⁴⁰]exendin-4, [His²⁰,Arg^{40]exendin-}4; and [Leu¹⁴,Lys²⁰,Arg⁴⁰]exendin-4.

The invention may also use truncated forms of exendin-4 or any of the exendin analogs described herein. The truncated forms may include deletions of 1, 2, 3, 4, 5, 6, 7, 8, 9, 10, 11, 12, 13, 14, 15, 16, 17, 18, 19, or 20 amino acids from the N-terminus, from the C-terminus, or a combination thereof. Particular exendin-4 fragments include Exendin-4(1-31). Other fragments of exendin-4 are described in U.S. Patent Application Publication No. 2007/0037747 and have the formula:

His-Gly-Glu-Gly-Thr-X₆-Thr-Ser-Asp-Leu-Ser-Lys-Gln-X₁₄-Glu-Glu-Glu-Ala-Val-X₂₀-
Leu-Phe-Ile-Glu-Trp-Leu-Lys-Asn-Gly-X₃₀-Pro-X₃₂

where X₆ is Phe or Tyr; X₁₄ is Met, Ile, or Leu; X₂₀ is Lys; X₃₀ is Gly or is absent; and X₃₂ is Arg or is absent.

GLP-1 and GLP-1 analogs. The GLP-1 agonist used in the compositions and methods of the invention can be GLP-1 or a GLP-1 analog. In certain embodiments, the GLP-1 analog is a polypeptide, which can be truncated, may have one or more substitutions of the wild type sequence (e.g., the human wild type sequence), or may have other chemical modifications. GLP-1 agonists can also be non-peptide compounds, for example, as described in U.S. Pat. No. 6,927,214. Particular analogs include LY548806, CJC-1131, and Liraglutide.

The GLP-1 analog can be truncated form of GLP-1. The GLP-1 polypeptide may be truncated by 1, 2, 3, 4, 5, 6, 7, 8, 9, 10, 11, 12, 13, 15, 20, or more residues from its N-terminus, its C-terminus, or a combination thereof. In certain embodiments, the truncated GLP-1 analog is the GLP-1(7-34), GLP-1(7-35), GLP-1(7-36), or GLP-1(7-37) human polypeptide or the C-terminal amidated forms thereof.

In other embodiments of the invention, modified forms of truncated GLP-1 peptides are used. Exemplary analogs are described in U.S. Pat. No. 5,545,618 and have the amino acid sequence:

His-Ala-Glu-Gly-Thr-Phe-Thr-Ser-Asp-Val-Ser-Ser-Tyr-Leu-Glu-Gly-Gln-Ala-Ala-Lys-
Glu-Phe-Ile-Ala-Trp-Leu-Val-Lys-(Gly)-(Arg)-(Gly)

where (Gly), (Arg), and (Gly) are present or absent depending on indicated chain length, with at least one modification selected from the group consisting of (a) substitution of a neutral amino acid, Arg, or a D form of Lys for Lys at position 26 and/or 34 and/or a neutral amino acid, Lys, or a D form of Arg for Arg at position 36; (b) substitution of an oxidation-resistant amino acid for Trp at position 31; (c) substitution according to at least one of: Tyr for Val at position 16; Lys for Ser at position 18; Asp for Glu at position 21; Ser for Gly at position 22; Arg for Gln at position 23; Arg for Ala at position 24; and Gln for Lys at position 26; (d) a substitution comprising at least one of an alternative small neutral amino acid for Ala at position 8; an alternative acidic amino acid or neutral amino acid for Glu at position 9; an alternative neutral amino acid for Gly at position 10; and an alternative acidic amino acid for Asp at position 15; and (e) substitution of an alternative neutral amino acid or the Asp or N-acylated or alkylated form of H is for His at position 7. With respect to modifications (a), (b), (d), and (e), the substituted amino acids may be in the D form. The amino acids substituted at position 7 can also be the N-acylated or N-alkylated amino acids. Exemplary GLP-1 analogs include [D-His⁷]GLP-1(7-37), [Tyr⁷]GLP-1(7-37), [N-acetyl-His⁷]GLP-1(7-37), [N-isopropyl-His⁷]GLP-1(7-37), [D-Ala⁸]GLP-1(7-37), [D-Glu⁹]GLP-1(7-37), [Asp⁹]GLP-1(7-37), [D-Asp⁹]GLP-1(7-37), [D-Phe¹⁰]GLP-1(7-37), [Ser²²,Arg²³,Arg²⁴,Gln²⁶]GLP-1(7-37), and [Ser⁸,Gln⁹,Tyr¹⁶,Lys¹⁸,Asp²¹]GLP-1(7-37).

Other GLP-1 fragments are described in U.S. Pat. No. 5,574,008 have the formula:

R₁-Ser-Tyr-Leu-Glu-Gly-Gln-Ala-Ala-Lys-Glu-Phe-Ile-Ala-Trp-Leu-Val-X-Gly-Arg-R₂

where R₁ is H₂N; H₂N-Ser; H₂N-Val-Ser; H₂N-Asp-Val-Ser; H₂N-Ser-Asp-Val-Ser; H₂N-Thr-Ser-Asp-Val-Ser; H₂N-Phe-Thr-Ser-Asp-Val-Ser; H₂N-Thr-Phe-Thr-Ser-Asp-Val-Ser; H₂N-Gly-Thr-Phe-Thr-Ser-Asp-Val-Ser; H₂N-Glu-Gly-Thr-Phe-Thr-Ser-Asp-Val-Ser; or H₂N-Ala-Glu-Gly-Thr-Phe-Thr-Ser-Asp-Val-Ser; X is Lys or Arg; and R₂ is NH₂, OH, Gly-NH₂, or Gly-OH.

Other GLP-1 analogs, described in U.S. Pat. No. 5,118,666, include the sequence His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-X, where X is Lys, Lys-Gly, or Lys-Gly-Arg.

GLP-1 analogs also include peptides of the formula: H₂N—X—CO—R₁, where R₁ is OH, OM, or —NR₂R₃; M is a pharmaceutically acceptable cation or a lower branched or unbranched alkyl group (e.g., C_{I} alkyl); R₂ and R₃ are independently selected from the group consisting of hydrogen and a lower branched or unbranched alkyl group (e.g., C₁₋₆ alkyl); X is a polypeptide comprising the sequence His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys-Gly-Arg; NH₂ is the amine group of the amino terminus of X; and CO is the carbonyl group of the carboxy terminus of X; acid addition salts thereof; and the protected or partially protected derivatives thereof. These compounds may have insulinotropic activity exceeding that of GLP-1(1-36) or GLP-1(1-37).

Other GLP-1 analogs are described in U.S. Pat. No. 5,981,488 and have the formula:

R₁-X-Glu-Gly-Thr-Phe-Thr-Ser-Asp-Val-Ser-Ser-Tyr-Leu-Y-Gly-Gln-Ala-Ala-Lys-Z-Phe-
Ile-Ala-Trp-Leu-Val-Lys-Gly-Arg-R₂

where R₁ is His, D-His, desamino-His, 2-amino-His, β-hydroxy-His, homohistidine, α-fluoromethyl-His, or α-methyl-His; X is Met, Asp, Lys, Thr, Leu, Asn, Gln, Phe, Val, or Tyr; Y and Z are independently selected from Glu, Gln, Ala, Thr, Ser, and Gly; and R₂ is selected from NH₂ and Gly-OH (e.g., provided that, if R₁ is His, X is Val, Y is Glu, and Z is Glu, then R₂ is NH₂).

Other GLP-1 analogs are described in U.S. Pat. No. 5,512,549 and have the formula:

R₁-Ala-Glu-Gly-Thr-Phe-Thr-Ser-Asp-Val-Ser-Ser-Tyr-Leu-Glu-Gly-Gln-Ala-Ala-Xaa-
Glu-Phe-Ile-Ala-Trp-Leu-Val-Lys(R₂)-Gly-Arg-R₃

where R₁ is 4-imidazopropionyl (des-amino-histidyl), 4-imidazoacetyl, or 4-imidazo-α, αdimethyl-acetyl; R₂, which is bound to the side chain of the Lys (e.g., through the c amino group), is C₆₋₁₀ unbranched acyl or is absent; R₃ is Gly-OH or NH₂; and Xaa is Lys or Arg.

Still other GLP-1 analogs are described in U.S. Pat. No. 7,084,243. In one embodiment, the GLP-1 analog has the formula:

His-X₈-Glu-Gly-X₁₁-X₁₂-Thr-Ser-Asp-X₁₆-Ser-Ser-Tyr-Leu-Glu-X₂₂-X₂₃-X₂₄-Ala-X₂₆-X₂₇-
Phe-Ile-Ala-X₃₁-Leu-X₃₃-X₃₄-X₃₅-X₃₆-R

where X₈ is Gly, Ala, Val, Leu, Ile, Ser, or Thr; X₁₁ is Asp, Glu, Arg, Thr, Ala, Lys, or His; X₁₂ is His, Trp, Phe, or Tyr; X₁₆ is Leu, Ser, Thr, Trp, His, Phe, Asp, Val, Tyr, Glu, or Ala; X₂₂ is Gly, Asp, Glu, Gln, Asn, Lys, Arg, Cys, or Cya; X₂₃ is His, Asp, Lys, Glu, or Gln; X₂₄ is Glu, His, Ala, or Lys; X₂₆ is Asp, Lys, Glu, or His; X₂₇ is Ala, Glu, His, Phe, Tyr, Trp, Arg, or Lys; X₃₀ is Ala, Glu, Asp, Ser, or His; X₃₃ is Asp, Arg, Val, Lys, Ala, Gly, or Glu; X₃₄ is Glu, Lys, or Asp; X₃₅ is Thr, Ser, Lys, Arg, Trp, Tyr, Phe, Asp, Gly, Pro, His, or Glu; X₃₆ is Arg, Glu, or His; R is Lys, Arg, Thr, Ser, Glu, Asp, Trp, Tyr, Phe, His, —NH₂, Gly, Gly-Pro, or Gly-Pro-NH₂, or is deleted (e.g., provided that the polypeptide does not have the sequence of GLP-1(7-37)OH or GLP-1(7-36)-NH₂ and provided that the polypeptide is not Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser^{a}-GLP-1(7-37)OH, Ser^{b}-GLP-1(7-36)NH₂, Thr⁸-GLP-1(7-37)OH, Thr⁸-GLP-1(7-36)NH₂, Ala¹¹-GLP-1(7-37)OH, Ala¹¹-GLP-1(7-36)NH₂, Ala¹⁶-GLP-1(7-37)OH, Ala¹⁶-GLP-1(7-36)NH₂, Ala²⁷-GLP-1(7-37)OH, Ala²⁷-GLP-1(7-36)NH₂, Ala²⁷-GLP-1(7-37)OH, Ala²⁷-GLP-1(7-36)NH₂, Ala³³-GLP-1(7-37)OH, or Ala³³-GLP-1(7-36)NH₂).

In another embodiment, the polypeptide has the amino acid sequence:

His-X₈-Glu-Gly-Thr-X₁₂-Thr-Ser-Asp-X₁₆-Ser-Ser-Tyr-Leu-Glu-X₂₂-X₂₃-Ala-Ala-X₂₆-Glu-
Phe-Ile-X₃₀-Trp-Leu-Val-Lys-X₃₅-Arg-R

where X₈ is Gly, Ala, Val, Leu, Ile, Ser, or Thr; X₁₂ is His, Trp, Phe, or Tyr; X₁₆ is Leu, Ser, Thr, Trp, His, Phe, Asp, Val, Glu, or Ala; X₂₂ is Gly, Asp, Glu, Gln, Asn, Lys, Arg, Cys, or Cya; X₂₃ is His, Asp, Lys, Glu, or Gin; X₂₆ is Asp, Lys, Glu, or His; X₃₀ is Ala, Glu, Asp, Ser, or His; X₃₅ is Thr, Ser, Lys, Arg, Trp, Tyr, Phe, Asp, Gly, Pro, His, or Glu; R is Lys, Arg, Thr, Ser, Glu, Asp, Trp, Tyr, Phe, His, —NH₂, Gly, Gly-Pro, Gly-Pro-NH₂, or is deleted, (e.g., provided that the polypeptide does not have the sequence of GLP-1(7-37)OH or GLP-1(7-36)NH₂ and provided that the polypeptide is not Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser^{b}-GLP-1(7-37)OH, Ser⁸-GLP-1(7-36)NH₂, Thr⁸-GLP-1(7-37)OH, Thr⁸-GLP-1(7-36)NH₂, Ala¹⁶-GLP(7-37)OH, or Ala¹⁶-GLP-1(7-36)NH₂).

In another embodiment, the polypeptide has the amino acid sequence:

His-X₈-Glu-Gly-Thr-Phe-Thr-Ser-Asp-Val-Ser-Ser-Tyr-Leu-Glu-X₂₂-X₂₃-Ala-Ala-Lys-X₂₇-
Phe-Ile-X₃₀-Trp-Leu-Val-Lys-Gly-Arg-R

where X₈ is Gly, Ala, Val, Leu, Ile, Ser, or Thr; X₂₂ is Gly, Asp, Glu, Gln, Asn, Lys, Arg, Cys, or Cya; X₂₃ is His, Asp, Lys, Glu, or Gln; X₂₇ is Ala, Glu, His, Phe, Tyr, Trp, Arg, or Lys X₃₀ is Ala, Glu, Asp, Ser, or His; R is Lys, Arg, Thr, Ser, Glu, Asp, Trp, Tyr, Phe, His, —NH₂, Gly, Gly-Pro, or Gly-Pro-NH₂, or is deleted (e.g., provided that the polypeptide does not have the sequence of GLP-1(7-37)011 or GLP-1(7-36)NH₂ and provided that the polypeptide is not Gly⁸-GLP-1(7-37)OH, Gly⁸-GLP-1(7-36)NH₂, Val⁸-GLP-1(7-37)OH, Val⁸-GLP-1(7-36)NH₂, Leu⁸-GLP-1(7-37)OH, Leu⁸-GLP-1(7-36)NH₂, Ile⁸-GLP-1(7-37)OH, Ile⁸-GLP-1(7-36)NH₂, Ser⁸-GLP-1(7-37)OH, Ser^{b}-GLP-1(7-36)NH₂, Thr⁸-GLP-1(7-37)OH, Thr⁸-GLP-1(7-36)NH₂, Ala¹⁶-GLP-1(7-37)OH, Ala¹⁶-Glp-1(7-36) NH₂, Glu²⁷-Glp-1(7-37)OH, or Glu²⁷-Glp-1(7-36)NH₂.

In another embodiment, the polypeptide has the amino acid sequence:

X₇-X₈-Glu-Gly-Thr-Phe-Thr-Ser-Asp-Val-Ser-Ser-Tyr-Leu-Glu-X₂₂-Gln-Ala-Ala-Lys-Glu-
Phe-Ile-Ala-Trp-Leu-Val-Lys-Gly-Arg-R

where X₇ is L-His, D-His, desamino-His, 2-amino-His, β-hydroxy-His, homo-His, α-fluoromethyl-His or α-methyl-His; X₈ is Gly, Ala, Val, Leu, Ile, Ser or Thr (e.g., Gly, Val, Leu, Ile, Ser, or Thr); X₂₂ is Asp, Glu, Gln, Asn, Lys, Arg, Cys, or Cya, and R is —NH₂ or Gly(OH).

In another embodiment, the GLP-1 compound has an amino acid other than alanine at position 8 and an amino acid other than glycine at position 22. Specific examples of GLP-1 compounds include [Glu²²]GLP-1(7-37)OH, [Asp²²]GLP-1(7-37)OH, [Arg²²]GLP-1(7-37)OH, [Lys²²]GLP-1(7-37)OH, [Cya²²]GLP-1(7-37)OH, [Val⁸,Glu²²]GLP-1(7-37)OH, [Val⁸,Asp²²]GLP-1(7-37)OH, [Val⁸,Arg²²]GLP-1(7-37)OH, [Val⁸,Lys²²]GLP-1(7-37)OH, [Val⁸,Cya²²]GLP-1(7-37)OH, [Gly⁸,Glu²²]GLP-1(7-37)OH, [Gly⁸,Asp²²]GLP-1(7-37)OH, [Gly⁸,Arg²²]GLP-1(7-37)OH, [Gly⁸,Lys²²]GLP-1(7-37)OH, [Gly⁸,Cya²²]GLP-1(7-37)OH, [Glu²²]GLP-1(7-36)NH₂, [Asp²²]GLP-1(7-36)NH₂, [Arg²²]GLP-1(7-36)NH₂, [Lys²²]GLP-1(7-36)NH₂, [Cya²²]GLP-1(7-36)NH₂, [Val⁸,Glu²²]GLP-1(7-36)NH₂, [Val⁸,Asp²²]GLP-1(7-36)NH₂, [Val⁸,Arg²²]GLP-1(7-36)NH₂, [Val⁸,Lys²²]GLP-1(7-36)NH₂, [Val⁸,Cya²²]GLP-1(7-36)NH₂, [Gly⁸,Glu²²]GLP-1(7-36)NH₂, [Gly⁸,Asp²²]GLP-1(7-36)NH₂, [Gly⁸,Arg²²]GLP-1(7-36)NH₂, [Gly⁸,Lys²²]GLP-1(7-36)NH₂, [Gly⁸,Cya²²]GLP-1(7-36)NH₂, [Val⁸,Lys²³]GLP-1(7-37)OH, [Val⁸,Ala²¹]GLP-1(7-37)OH, [Val⁸,Glu³⁰]GLP-1(7-37)OH, [Gly⁸,Glu³⁰]GLP-1(7-37)OH, [Val⁸,His³⁵]GLP-1(7-37)OH, [Val⁸,His³⁷]GLP-1(7-37)OH, [Val⁸, Glu²²,Lys²²]GLP-1(7-37)OH, [Val⁸,Glu²²,Glu²]GLP-1(7-37)OH, [Val⁸,Glu²²,Ala²⁷]GLP-1(7-37)OH, [Val⁸,Gly³⁴,Lys³⁵]GLP-1(7-37)OH, [Val⁸,His³⁷]GLP-1(7-37)OH, or [Gly⁸,His³⁷]GLP-1(7-37)OH.

Other GLP-1 analogs are described in U.S. Pat. No. 7,101,843 and include those having the formula:

X₇-X₈-Glu-Gly-Thr-X₁₂-Thr-Ser-Asp-X₁₆-Ser-X₁₈-X₁₉-X₂₀-Glu-X₂₂-Gln-Ala-X₂₅-Lys-X₂₇-
Phe-Ile-X₃₀-Trp-Leu-X₃₃-Lys-Gly-Arg-X₃₇

wherein: X₇ is L-His, D-His, desamino-His, 2-amino-His, β-hydroxy-His, homohistidine, α-fluoromethyl-His, or α-methyl-His; X₈ is Ala, Gly, Val, Leu, Ile, Ser, or Thr; X₁₂ is Phe, Trp, or Tyr; X₁₆ is Val, Trp, Ile, Leu, Phe, or Tyr; X₁₈ is Ser, Trp, Tyr, Phe, Lys, Ile, Leu, or Val; X₁₉ is Tyr, Trp, or Phe; X₂₀ is Leu, Phe, Tyr, or Trp; X₂₂ is Gly, Glu, Asp, or Lys; X₂₅ is Ala, Val, Ile, or Leu; X₂₇ is Glu, Ile, or Ala; X₃₀ is Ala or Glu X₃₃ is Val, or Ile; and X₃₇ is Gly, His, NH₂, or is absent (e.g., provided that the compound does not have the sequence GLP-1(7-37)OH, GLP-1(7-36)-NH₂, [Gly⁸]GLP-1(7-37)OH, [Gly⁸]GLP-1(7-36)NH₂, [Val⁸]GLP-1(7-37)OH, [Val⁸]GLP-1(7-36)NH₂, [Leu⁸]GLP-1(7-37)OH, [Leu⁸]GLP-1(7-36)NH₂, [Ile⁸]GLP-1(7-37)OH, [Ile⁸]GLP-1(7-36)NH₂, [Ser⁸]GLP-1(7-37)OH, [Ser⁸]GLP-1(7-36)NH₂, [Thr⁸]GLP-1(7-37)OH, [Thr⁸]GLP-1(7-36)NH₂, [Val⁸,Tyr¹²]GLP-1(7-37)OH, [Val⁸,Tyr¹²]GLP-1(7-36)NH₂, [Val⁸,Tyr¹²]GLP-1(7-37)OH, [Val⁸,Tyr¹⁶]GLP-1(7-36)NH₂, [Val⁸,Glu²²]GLP-1(7-37)OH, [Val⁸,Glu²²]GLP-1(7-36)NH₂, [Gly⁸,Glu²²]GLP-1(7-37)OH, [Gly⁸,Glu²²]GLP-1(7-36)NH₂, [Val⁸,Asp²²]GLP-1(7-37)OH, [Val⁸,Asp²²]GLP-1(7-36)NH₂, [Gly⁸,Asp²²]GLP-1(7-37)OH, [Gly⁸,Asp²²]GLP-1(7-36)NH₂, [Val⁸,Lys²²]GLP-1(7-37)OH, [Val⁸,Lys²²]GLP-1(7-36)NH₂, [Gly⁸,Lys²²]GLP-1(7-37)OH, [Gly⁸,Lys²²]GLP-1(7-36)NH₂, [Leu⁸,Glu²²]GLP-1(7-37)OH, [Leu⁸,Glu²²]GLP-1(7-36)NH₂, [Ile⁸,Glu²²]GLP-1(7-37)OH, [Ile⁸,Glu²²]GLP-1(7-36)NH₂, [Leu⁸,Asp²²]GLP-1(7-37)OH, [Leu⁸,Asp²²]GLP-1(7-36)NH₂, [Ile⁸,Asp²²]GLP-1(7-37)OH, [Ile⁸,Asp²²]GLP-1(7-36)NH₂, [Leu⁸,Lys²²]GLP-1(7-37)OH, [Leu⁸,Lys²²]GLP-1(7-36)NH₂, [Ile⁸,Lys²²]GLP-1(7-37)OH, [Ile⁸,Lys²²]GLP-1(7-36)NH₂, [Ser⁸,Glu²²]GLP-1(7-37)OH, [Ser⁸,Glu²²]GLP-1(7-36)NH₂, [Thr⁸,Glu²²]GLP-1(7-37)OH, [Thr⁸,Glu²²]GLP-1(7-36)NH₂, [Ser⁸,Asp²²]GLP-1(7-37)OH, [Ser⁸,Asp²²]GLP-1(7-36)NH₂, [Thr⁸,Asp²²]GLP-1(7-37)OH, [Thr⁸,Asp²²]GLP-1(7-36)NH₂, [Ser⁸,Lys²²]GLP-1(7-37)OH, [Ser⁸,Lys²²]GLP-1(7-36)NH₂, [Thr⁸,Lys²²]GLP-1(7-37)OH, [Thr⁸,Lys²²]GLP-1(7-36)NH₂, [Glu²²]GLP-1(7-37)OH, [Glu²]GLP-1(7-36)NH₂, [Asp²²]GLP-1(7-37)OH, [Asp²²]GLP-1(7-36)NH₂, [Lys²²]GLP-1(7-37)OH, [Lys²²]GLP-1(7-36)NH₂, [Val⁸,Ala²⁷]GLP-1(7-37)OH, [Val⁸,Glu²²,Ala²⁷]GLP-1(7-37)OH, [Val⁸,Glu³⁰]GLP-1(7-37)OH, [Val⁸,Glu³⁰]GLP-1(7-36)NH₂, [Gly⁸,Glu³⁰]GLP-1(7-37)OH, [Gly⁸,Glu³⁰]GLP-1(7-36)NH₂, [Leu⁸,Glu³⁰]GLP-1(7-37)OH, [Leu⁸,Glu³⁰]GLP-1(7-36)NH₂, [Ile⁸,Glu³⁰]GLP-1(7-37)OH, [Ile⁸,Glu³⁰]GLP-1(7-36)NH₂, [Ser⁸,Glu³⁰]GLP-1(7-37)OH, [Ser⁸,Glu³⁰]GLP-1(7-36)NH₂, [Thr⁸,Glu³⁰]GLP-1(7-37)OH, [Thr⁸,Glu³⁰]GLP-1(7-36)NH₂, [Val⁸,His³⁷]GLP-1(7-37)OH, [Val⁸,His³⁷]GLP-1(7-36)NH₂, [Gly⁸,His³⁷]GLP-1(7-37)OH, [Gly⁸,His³⁷]GLP-1(7-36)NH₂, [Leu⁸,His³⁷]GLP-1(7-37)OH, [Leu⁸,His³⁷]GLP-1(7-36)NH₂, [Ile⁸,His³⁷]GLP-1(7-37)OH, [Ile⁸,His³⁷]GLP-1(7-36)NH₂, [Ser⁸,His³⁷]GLP-1(7-37)OH, [Ser⁸,His³⁷]GLP-1(7-36)NH₂, [Thr⁸,His³⁷]GLP-1(7-37)OH, or [Thr⁸,His³⁷]GLP-1(7-36)NH₂).

Other GLP-1 analogs described in U.S. Pat. No. 7,101,843 have the formula:

X₇-X₈-Glu-Gly-Thr-Phe-Thr-Ser-Asp-X₁₆-Ser-X₁₈-Tyr-Leu-Glu-X₂₂-Gln-Ala-X₂₅-
Lys-Glu-Phe-Ile-Ala-Trp-Leu-X₃₃-Lys-Gly-Arg-X₃₇

wherein: X₇ is L-His, D-His, desamino-His, 2-amino-His, β-hydroxy-His, homohistidine, α-fluoromethyl-His, or α-methyl-His; X₈ is Gly, Ala, Val, Leu, Ile, Ser, or Thr; X₁₆ is Val, Phe, Tyr, or Trp; X₁₈ is Ser, Tyr, Trp, Phe, Lys, Ile, Leu, or Val; X₂₂ is Gly, Glu, Asp, or Lys; X₂₅ is Ala, Val, Ile, or Leu; X₃₃ is Val or Ile; and X₃₇ is Gly, NH₂, or is absent (e.g., provided that the GLP-1 compound does not have the sequence of GLP-1(7-37)OH, GLP-1(7-36)-NH₂, [Gly⁸]GLP-1(7-37)OH, [Gly⁸]GLP-1(7-36)NH₂, [Val⁸]GLP-1(7-37)OH, [Val⁸]GLP-1(7-36)NH₂, [Leu⁸]GLP-1(7-37)OH, [Leu⁸]GLP-1(7-36)NH₂, [Ile⁸]GLP-1(7-37)OH, [Ile⁸]GLP-1(7-36)NH₂, [Ser⁸]GLP-1(7-37)OH, [Ser⁸]GLP-1(7-36)NH₂, [Thr⁸]GLP-1(7-37)OH, [Thr⁸]GLP-1(7-36)NH₂, [Val⁸-Tyr¹⁶]GLP-1(7-37)OH, [Val⁸-Tyr¹⁶]GLP-1(7-36)NH₂, [Val⁸,Glu²²]GLP-1(7-37)OH, [Val⁸,Glu²²]GLP-1(7-36)NH₂, [Gly⁸,Glu²²]GLP-1(7-37)OH, [Gly⁸,Glu²²]GLP-1(7-36)NH₂, [Val⁸,Asp²²]GLP-1(7-37)OH, [Val⁸,Asp²²]GLP-1(7-36)NH₂, [Gly⁸,Asp²²]GLP-1(7-37)OH, [Gly⁸,Asp²²]GLP-1(7-36)NH₂, [Val⁸,Lys²²]GLP-1(7-37)OH, [Val⁸,Lys²²]GLP-1(7-36)NH₂, [Gly⁸,Lys²²]GLP-1(7-37)OH, [Gly⁸,Lys²²]GLP-1(7-36)NH₂, [Leu⁸,Glu²²]GLP-1(7-37)OH, [Leu⁸,Glu²²]GLP-1(7-36)NH₂, [Ile⁸,Glu²²]GLP-1(7-37)OH, [Ile⁸,Glu²²]GLP-1(7-36)NH₂, [Leu⁸,Asp²²]GLP-1(7-37)OH, [Leu⁸,Asp²²]GLP-1(7-36)NH₂, [Ile⁸,Asp²²]GLP-1(7-37)OH, [Ile⁸,Asp²²]GLP-1(7-36)NH₂, [Leu⁸,Lys²²]GLP-1(7-37)OH, [Leu⁸,Lys²²]GLP-1(7-36)NH₂, [Ile⁸,Lys²²]GLP-1(7-37)OH, Ile⁸,Lys²²]GLP-1(7-36)NH₂, [Ser⁸,Glu²²]GLP-1(7-37)OH, [Ser⁸,Glu²²]GLP-1(7-36)NH₂, [Thr⁸,Glu²²]GLP-1(7-37)OH, [Thr⁸,Glu²²]GLP-1(7-36)NH₂, [Ser⁸,Asp²²]GLP-1(7-37)OH, [Ser⁸,Asp²²]GLP-1(7-36)NH₂, [Thr⁸,Asp²²]GLP-1(7-37)OH, [Thr⁸,Asp²²]GLP-1(7-36)NH₂, [Ser⁸,Lys²²]GLP-1(7-37)OH, [Ser⁸,Lys²²]GLP-1(7-36)NH₂, [Thr⁸,Lys²²]GLP-1(7-37)OH, [Thr⁸ μLys²²]GLP-1(7-36)NH₂, [Glu²²]GLP-1(7-37)OH, [Glu²²]GLP-1(7-36)NH₂, [Asp²²]GLP-1(7-37)OH, [Asp²²]GLP-1(7-36)NH₂, [Lys²²]GLP-1(7-37)OH, or [Lys²²]GLP-1(7-36)NH₂).

GLP-1 analogs are also described in U.S. Pat. No. 7,238,670 and have the structure:

A-X₁-X₂-X₃-X₄-X₅-X₆-X₇-X₈-X₉-Y-Z-B

where each of X₁-X₉ is a naturally or nonnaturally occurring amino acid residue; Y and Z are amino acid residues; and one of the substitutions at the α-carbon atoms of Y and Z may each independently be substituted with a primary substituent group selected from the group consisting of hydrogen, alkyl, cycloalkyl, cycloalkylalkyl, heterocyclylalkyl, arylalkyl and heteroarylalkyl, heterocyclylalkyl said primary substituent optionally being substituted with a secondary substituent selected from a cycloalkyl, heterocyclyl, aryl, or heteroaryl group; any of said primary or secondary substituents may further be substituted with one or more of H, alkyl, cycloalkyl, arylalkyl, aryl, heterocyclyl, heteroaryl, alkenyl, alkynyl, halo, hydroxy, mercapto, nitro, cyano, amino, acylamino, azido, guanidine, amidino, carboxyl, carboxamido, carboxamido alkyl, formyl, acyl, carboxyl alkyl, alkoxy, aryloxy, arylalkyloxy, heteroaryloxy, heterocycleoxy, acyloxy, mercapto, mercapto alkyl, mercaptoaryl, mercapto acyl, halo, cyano, nitro, azido, amino, guanidino alkyl, guanidino acyl, sulfonic, sulfonamido, alkyl sulfonyl, aryl sulfonyl or phosphonic group; wherein, the primary or secondary substitutents may optionally be bridged by covalent bonds to form one or more fused cyclic or heterocyclic systems with each other; where, the other substitution at the alpha-carbon of Y may be substituted with H, C₁₋₆ alkyl, aminoalkyl, hydroxyalkyl or carboxyalkyl; where the other substitution at the alpha-carbon of Z may be substituted with hydrogen, C₁₋₁₂ alkyl, aminoalkyl, hydroxyalkyl, or carboxyalkyl;

A and B are optionally present, where A is present and A is H, an amino acid or polypeptide containing from about 1-15 amino acid residues, an R group, an R—C(O) (amide) group, a carbamate group RO—C(O), a urea R₄R₅N—C(O), a sulfonamido R—SO₂, or R₄R₅N—SO₂; where R is selected from the group consisting of hydrogen, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, and heteroaryloxyalkyl; R₄ and R₅ are each independently selected from the group consisting of H, alkyl, cycloalkyl, cycloalkylalkyl, heterocyclyl, heterocycloalkyl, aryl, heteroaryl, arylalkyl, aryloxyalkyl, heteroarylalkyl, and heteroaryloxyalky; where the α-amino group of X₁ is substituted with H or an alkyl group, said alkyl group may optionally form a ring with A; where B is present and B is OR₁, NR₁R₂, or an amino acid or polypeptide containing from 1 to 15 amino acid residues (e.g., 1 to 10 or 1 to 5) terminating at the C-terminus as a carboxamide, substituted carboxamide, an ester, a free carboxylic acid, or an amino-alcohol; where R₁ and R₂ are independently chosen from H, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, or heteroaryloxyalkyl.

Exemplary substitutions on the α-carbon atoms of Y and Z include heteroarylarylmethyl, arylheteroarylmethyl, and biphenylmethyl forming biphenylalanine residues, any of which is also optionally substituted with one or more, hydrogen, alkyl, cycloalkyl, arylalkyl, aryl, heterocyclyl, heteroaryl, alkenyl, alkynyl, halo, hydroxy, mercapto, nitro, cyano, amino, acylamino, azido, guanidino, amidino, carboxyl, carboxamido, carboxamido alkyl, formyl, acyl, carboxyl alkyl, alkoxy, aryloxy, arylalkyloxy, heteroaryloxy, heterocycleoxy, acyloxy, mercapto, mercapto alkyl, mercaptoaryl, mercapto acyl, halo, cyano, nitro, azido, amino, guanidino alkyl, guanidino acyl, sulfonic, sulfonamido, alkyl sulfonyl, aryl sulfonyl, and phosphonic group. Other embodiments include isolated polypeptides where the other substitution at the α-carbon of Y is substituted with H, methyl, or ethyl; and where the other substitution at the α-carbon of Z is substituted with H, methyl, or ethyl.

Further embodiments include isolated polypeptides as described above, where X₁ is naturally or non-naturally occurring amino acid residue in which one of the substitutions at the α-carbon is a primary substituent selected from the group consisting of heterocyclylalkyl, heteroaryl, heteroarylkalkyl and arylalkyl, said primary substituent optionally being substituted with secondary substituent selected from heteroaryl or heterocyclyl; and in which the other substitution at the α-carbon is H or alkyl; X, is naturally or nonnaturally occurring amino acid residue in which one of the substitutions at the α-carbon is an alkyl or cycloalkyl where the alkyl group may optionally form a ring with the nitrogen of X₂; and where the other substitution at the α-carbon is H or alkyl; X₃ is a naturally or nonnaturally occurring amino acid residue in which one of the substitutions at the α-carbon is a carboxyalkyl, bis-carboxyalkyl, sulfonylalkyl, heteroalkyl, or mercaptoalkyl; and where the other substitution at the α-carbon is hydrogen or alkyl; X₄ is a naturally or nonnaturally occurring amino acid residue in which the α-carbon is not substituted, or in which one of the substitutions at the α-carbon is aminoalkyl, carboxyalkyl heteroarylalkyl, or heterocycylalkyl; X₅ is a naturally or nonnaturally occurring amino acid residue in which one of the substitutions at the α-carbon is an alkyl or hydroxyalkyl, and in which the other substitution at the α-carbon is hydrogen or alkyl; X₆ is a naturally or nonnaturally occurring amino acid residue in which one of the substitutions at the α-carbon is C₁₋₁₂ alkyl, aryl, heteroaryl, heterocyclyl, cycloalkylalkyl, heterocyclylalkyl, arylalkyl, or heteroarylalkyl group, and the other substitution at the α-carbon is H or alkyl; X₇ is a naturally or nonnaturally occurring amino acid residue in which one of the substitutions at the α-carbon is a hydroxylalkyl group; X₈ is a naturally or nonnaturally occurring amino acid residue in which one of the substitutions at the α-carbon is C₁₋₁₂ alkyl, hydroxylalkyl, heteroarylalkyl, or carboxamidoalkyl, and the other substitution at the α-carbon is H or alkyl; X₉ is a naturally or nonnaturally occurring amino acid residue in which one of the substitutions at α-carbon is carboxylalkyl, bis-carboxylalkyl, carboxylaryl, sulfonylalkyl, carboxylamidoalkyl, or heteroarylalkyl; and where A is H, an amino acid or polypeptide containing from about 1 to about 5 amino acid residues, an R group, an R—C(O) amide group, a carbamate group RO—C(O), a urea R₄R₅N—C(O), a sulfonamido R—SO₂ or a R₄R₅N—SO₂.

In certain embodiments, X₁ is His, D-His, N-Methyl-His, D-N-Methyl-His, 4-ThiazolylAla, or D-4-ThiazolylAla; X₂ is Ala, D-Ala, Pro, Gly, D-Ser, D-Asn, Nma, D-Nma, 4-ThioPro, 4-Hyp, L-2-Pip, L-2-Azt, Aib, S— or R-Iva and Acc3; X₃ is Glu, N-Methyl-Glu, Asp, D-Asp, His, Gla, Adp, Cys, or 4-ThiazolyAla; X₄ is Gly, His, Lys, or Asp; X₅ is Thr, D-Thr, Nle, Met, Nva, or L-Aoc; X₆ is Phe, Tyr, Tyr(Bzl), Tyr(3-NO₂), Nle, Trp, Phe(penta-fluoro), D-Phe(penta-fluoro), Phe(2-fluoro), Phe(3-fluoro), Phe(4-fluoro), Phe(2,3-di-fluoro), Phe(3,4-di-fluoro), Phe(3,5-di-fluoro), Phe(2,6-di-fluoro), Phe(3,4,5-tri-fluoro), Phe(2-iodo), Phe(2-OH), Phe(2-OMe), Phe(3-OMe), Phe(3-cyano), Phe(2-chloro), Phe(2-NH₂), Phe(3-NH₂), Phe(4-NH₂), Phe(4-NO₂), Phe(4-Me), Phe(4-allyl), Phe(n-butyl), Phe(4-cyclohexyl), Phe(4-cyclohexyloxy), Phe(4-phenyloxy), 2-Nal, 2-pyridylAla, 4-thiazolylAla, 2-Thi, α-Me-Phe, D-α-Me-Phe, α-Et-Phe, D-α-Et-Phe, α-Me-Phe(2-fluoro), D-α-Me-Phe(2-fluoro), α-Me-Phe(2,3-di-fluoro), D-α-Me-Phe(2,3-di-fluoro), α-Me-Phe(2,6-di-fluoro), D-α-Me-Phe(2,6-di-fluoro), α-Me-Phe(penta-fluoro) and D-α-Me-Phe(penta-fluoro); X₇ is Thr, D-Thr, Ser, or hSer; X₈ is Ser, hSer, His, Asn, or α-Me-Ser; and X₉ is Asp, Glu, Gla, Adp, Asn, or His.

Additional embodiments include those where Y is Bip, D-Bip, L-Bip(2-Me), D-Bip(2-Me), L-Bip(2′-Me), L-Bip(2-Et), D-Bip(2-Et), L-Bip(3-Et), L-Bip(4-Et), L-Bip(2-n-propyl), L-Bip(2-n-propyl, 4-OMe), L-Bip(2-n-propyl,2′-Me), L-Bip(3-Me), L-Bip(4-Me), L-Bip(2,3-di-Me), L-Bip(2,4-di-Me), L-Bip(2,6-di-Me), L-Bip(2,4-di-Et), L-Bip(2-Me, 2′-Me), L-Bip(2-Et, 2′-Me), L-Bip(2-Et, 2′-Et), L-Bip(2-Me,4-OMe), L-Bip(2-Et,4-OMe), D-Bip(2-Et,4-OMe), L-Bip(3-OMe), L-Bip(4-OMe), L-Bip(2,4,6-tri-Me), L-Bip(2,3-di-OMe), L-Bip(2,4-di-OMe), L-Bip(2,5-di-OMe), L-Bip(3,4-di-OMe), L-Bip(2-Et,4,5-di-OMe), L-Bip(3,4-Methylene-di-oxy), L-Bip(2-Et, 4,5-Methylene-di-oxy), L-Bip(2-CH₂OH, 4-OMe), L-Bip(2-Ac), L-Bip(3-NH—Ac), L-Bip(4-NH—Ac), L-Bip(2,3-di-chloro), L-Bip(2,4-di-chloro), L-Bip(2,5-di-chloro), L-Bip(3,4-di-chloro), L-Bip(4-fluoro), L-Bip(3,4-di-fluoro), L-Bip(2,5-di-fluoro), L-Bip(3-n-propyl), L-Bip(4-n-propyl), L-Bip(2-iso-propyl), L-Bip(3-iso-propyl), L-Bip(4-iso-propyl), L-Bip(4-tert-butyl), L-Bip(3-phenyl), L-Bip(2-chloro), L-Bip(3-chloro), L-Bip(2-fluoro), L-Bip(3-fluoro), L-Bip(2-CF₃), L-Bip(3-CF₃), L-Bip(4-CF₃), L-Bip(3-NO₂), L-Bip(3-OCF₃), L-Bip(4-OCF₃), L-Bip(2-OEt), L-Bip(3-OEt), L-Bip(4-OEt), L-Bip(4-SMe), L-Bip(2-OH), L-Bip(3-OH), L-Bip(4-OH), L-Bip(2-CH₂—COOH), L-Bip(3-CH₂—COOH), L-Bip(4-CH₂—COOH), L-Bip(2-CH₂—NH₂), L-Bip(3-CH₂—NH₂), L-Bip(4-CH₂—NH₂), L-Bip(2-CH₂—OH), L-Bip(3-CH₂—OH), L-Bip(4-CH₂—OH), L-Phe[4-(1-propargyl)], L-Phe[4-(1-propenyl)], L-Phe[4-n-butyl], L-Phe[4-cyclohexyl], Phe(4-phenyloxy), L-Phe(penta-fluoro), L-2-(9,10-dihydrophenanthrenyl)-Ala, 4-(2-benzo(b)furan)-Phe, 4-(4-Dibenzofuran)-Phe, 4-(4-phenoxathiin)-Phe, 4-(2-Benzo(b)thiophene)-Phe, 4-(3-thiophene)-Phe, 4-(3-Quinoline)-Phe, 4-(2-naphthyl)-Phe, 4-(1-Naphthyl)-Phe, 4-(4-(3,5-dimethylisoxazole))-Phe, 4-(2,4-dimethoxypyrimidine)-Phe, homoPhe, Tyr(Bzl), Phe(3,4-di-chloro), Phe(4-Iodo), 2-Naphthyl-Ala, L-α-Me-Bip, or D-α-Me-Bip; Z is L-Bip, D-Bip, L-Bip(2-Me), D-Bip(2-Me), L-Bip(2′-Me), L-Bip(2-Et), D-Bip(2-Et), L-Bip(3-Me), L-Bip(4-Me), L-Bip(3-OMe), L-Bip(4-OMe), L-Bip(4-Et), L-Bip(2-n-propyl,2′-Me), L-Bip(2,4-di-Me), L-Bip(2-Me, 2′-Me), L-Bip(2-Me,4-OMe), L-Bip(2-Et, 4-OMe), D-Bip(2-Et,4-OMe), L-Bip(2,6-di-Me), L-Bip(2,4,6-tri-Me), L-Bip(2,3,4,5,-tetra-Me), L-Bip(3,4-di-OMe), L-Bip(2,5-di-OMe), L-Bip(3,4-methylene-di-oxy), L-Bip(3-NH—Ac), L-Bip(2-iso-propyl), L-Bip(4-iso-propyl), L-Bip(2-phenyl), L-Bip(4-phenyl), L-Bip(2-fluoro), L-Bip(4-CF₃), L-Bip(4-OCF₃), L-Bip(2-OEt), L-Bip(4-OEt), L-Bip(4-SMe), L-Bip(2-CH₂—COOH), D-Bip(2-CH₂—COOH), L-Bip(2′-CH₂—COOH), L-Bip(3-CH₂—COOH), L-Bip(4-CH₂—COOH), L-Bip(2-CH₂—NH₂), L-Bip(3-CH₂—NH₂), L-Bip(4-CH₂—NH₂), L-Bip(2-CH₂—OH), L-Bip(3-CH₂—OH), L-Bip(4-CH₂—OH), L-Phe(3-phenyl), L-Phe[4-n-butyl], L-Phe[4-cyclohexyl], Phe(4-phenyloxy), L-Phe(penta-fluoro), L-2-(9,10-dihydrophenanthrenyl)-Ala, 4-(3-pyridyl)-Phe, 4-(2-naphthyl)-Phe, 4-(1-naphthyl)-Phe, 2-naphthyl-Ala, 2-fluorenyl-Ala, L-α-Me-Bip, D-α-Me-Bip, L-Phe(4-NO₂), or L-Phe(4-iodo); A is H, acetyl, β-Ala, Ahx, Gly, Asp, Glu, Phe, Lys, Nva, Asn, Arg, Ser, Thr, Val, Trp, Tyr, caprolactam, Bip, Ser(Bzl), 3-pyridylAla, Phe(4-Me), Phe(penta-fluoro), 4-methylbenzyl, 4-fluorobenzyl, n-propyl, n-hexyl, cyclohexylmethyl, 6-hydroxypentyl, 2-thienylmethyl, 3-thienylmethyl, penta-fluorobenzyl, 2-naphthylmethyl, 4-biphenylmethyl, 9-anthracenylmethyl, benzyl, (S)-(2-amino-3-phenyl)propyl, methyl, 2-aminoethyl, or (S)-2-aminopropyl; and B is OH, NH₂, Trp-NH₂, 2-naphthylAla-NH₂, Phe(penta-fluoro)-NH₂, Ser(Bzl)-NH₂, Phe(4-NO₂)—NH₂, 3-pyridylAla-NH₂, Nva-NH₂, Lys-NH₂, Asp-NH₂, Ser-NH₂, His-NH₂, Tyr-NH₂, Phe-NH₂, L-Bip-NH₂, D-Ser-NH₂, Gly-OH, beta.-Ala-OH, GABA-OH, or APA-OH.

In certain embodiments, when A is not present, and X₁ is an R group, an R—C(O) (amide) group, a carbamate group RO—C(O), a urea R₄R₅N—C(O), a sulfonamido R—SO₂, or a R₄R₅N—SO₂; wherein R is H, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, heteroaryloxyalkyl, or heteroarylalkoxyalkyl; and where R₄ and R₅ are each independently H, C₁₋₁₂ alkyl, C₃₋₁₀ cycloalkyl, cycloalkylalkyl, heterocyclyl, heterocycloalkyl, aryl, heteroaryl, arylalkyl, aryloxyalkyl, heteroarylalkyl, or heteroaryloxyalky.

In certain embodiments, when B is not present and Z is OR₁, NR₁R₂, or an amino-alcohol; where R₁ and R₂ are independently H, C₁₋₁₂ alkyl, C₃₋₁₀ cycloalkyl, cycloalkylalkyl, heterocycle, heterocycloalkyl, aryl, heteroaryl, arylalkyl, aryloxyalkyl, heteroarylalkyl, or heteroaryloxyalkyl. In certain embodiments, X₁ (where applicable), X₂, and X₃ are N—H or N-alkylated, (e.g., N-methylated) amino acid residues. The polypeptide may be a 10-mer to 15-mer and capable of binding to and activating the GLP-1 receptor.

The following abbreviations are used above: Nal=naphthylalanine; pGly=pentylglycine; t-BuG=t-butylglycine; TPro=thioproline; HPro=homoproline; NmA=N-methylalanine; Cya=cysteic acid; Thi=/β2-Thienyl-Ala; hSer=homoserine; Aib=α-aminoisobutyric acid; Bip=biphenylalanine; Nle=norleucine; Ahx=2-aminohexanoic acid; and Nva=norvaline.

Leptin and Leptin Analogs

The transport vector used in the compositions and methods of the invention can also include leptin or a leptin derivative. Leptin is an adipokine, and thus the polypeptides used in the invention can include an adipokine or an analog thereof. Adipokines include adiponectin, leptin, and resistin. Adiponectins include human, mouse, and rat adiponectin. Leptins include leptin(116-130), leptin(22-56), leptin(57-92), leptin(93-105), LY396623, metreleptin, murine leptin analog, pegylated leptin, and methionyl human leptin. Resistins include human, mouse, and rat resistin. The leptin may be a cleaved sequence or the full-length protein. The polypeptide used in the invention may be any of these peptides or proteins or may be substantially identical to any of these peptides or proteins.

Neurotensin and Neurotensin Analogs

The compositions and methods of the invention can also include neurotensin (NT) or a NT analog. NT is a 13 amino acid polypeptide found in the central nervous system and in the gastrointestinal tract. In brain, NT is associated with dopaminergic receptors and other neurotransmitter system. Peripheral NT acts as a paracrine and endocrine polypeptide on both the digestive and cardiovascular systems. To exert its biological effects in the brain NT has to be injected or delivered directly to the brain because NT does not cross the BBB and is rapidly degraded by peptidases following systematic administration. Preclinical pharmacological studies, most of which involve direct injection of NT into the brain, strongly suggest that an agonist of NT receptors would be clinically useful for the treatment of neuropsychiatric conditions including psychosis, schizophrenia, Parkinson's disease, pain, and the abuse of psychostimulants. In particular, in various animal studies, intraventricular injection of NT led to hypothermia and analgesia in antinociception experiments.

Human neurotensin is a thirteen amino acid peptide having the sequence QLYENKPRRPYIL. Exemplary neurotensin analogs include (VIP-neurotensin) hybrid antagonist, acetylneurotensin(8-13), JMV 1193, KK13 peptide, neuromedin N, neuromedin N precursor, neurotensin(1-10), neurotensin(1-11), neurotensin(1-13), neurotensin(1-6), neurotensin(1-8), neurotensin(8-13), Asp(12)-neurotensin(8-13), Asp(13)-neurotensin(8-13), Lys(8)-neurotensin(8-13), N-methyl-Arg(8)-Lys(9)-neo-Trp(11)-neo-Leu(12)-neurotensin(8-13), neurotensin(9-13), neurotensin 69L, Arg(9)-neurotensin, azidobenzoyl-Lys(6)-Trp(11)-neurotensin, Gln(4)-neurotensin, iodo-Tyr(11)-neurotensin, iodo-Tyr(3)-neurotensin, N-α-(fluoresceinylthiocarbamyl)glutamyl(1)-neurotensin, Phe(11)-neurotensin, Ser(7)-neurotensin, Trp(11)-neurotensin, Tyr(11)-neurotensin, rat NT77, PD 149163, proneurotensin, stearyl-Nle(17)-neurotensin(6-11)VIP(7-28), ^{99m}Tc-NT-XI, TJN 950, and vasoactive intestinal peptide-neurotensin hybrid.

Other neurotensin analogs include NT64L [L-neo-Trp¹¹]NT(8-13), NT72D [D-Lys⁹,D-neo-Trp¹¹,tert-Leu¹²]NT(9-13), NT64D [D-neo-Trp¹¹]NT(8-13), NT73L [D-Lys⁹,L-neo-Trp¹¹]NT(9-13), NT65L [L-neo-Trp¹¹, tert-Leu¹²]NT(8-13), NT73D [D-Lys⁹,D-neo-Trp¹¹]NT(9-13), NT65D [D-neo-Trp¹¹, tert-Leu¹²]NT(8-13), NT74L [DAB⁹,L-neo-Trp¹¹,tert-Leu¹²]NT(9-13), NT66L [D-Lys⁸, L-neo-Trp¹¹, tert-Leu¹²]NT(8-13), NT74D [DAB⁹,Pro,D-neo-Trp¹¹,tert-Leu¹²]NT(9-13), NT66D [D-Lys⁸, D-neo-Trp¹¹, tert-Leu¹²]NT(8-13), NT75L [DAB8 L-neo-Trp¹¹]NT(8-13), NT67L [D-Lys⁸, L-neo-Trp¹¹]NT(8-13), NT75D [DAB⁸,D-neo-Trp¹¹]NT(8-13), NT67D [D-Lys⁸, D-neo-Trp¹¹,tert-Leu¹²]NT(8-13), NT76L [D-Orn⁹,D-neo-Trp¹¹]NT(8-13), NT69D[N-methyl-Arg⁸ L-Lys⁹ L-neo-Trp¹¹,tert-Leu¹²]NT(8-13), NT76D [D-Orn⁹,D-neo-Trp¹¹]NT(8-13), NT69D [N-methyl-Arg⁸ L-Lys⁹,D-neo-Trp¹¹,tert-Leu¹²]NT(8-13), NT77L [D-Orn⁹,L-neo-Trp¹¹,tert-Leu¹²]NT(8-13), NT71L [N-methyl-Arg⁸,DAB⁹ L-neo-Trp¹¹,tert-Leu¹²]NT(8-13), NT77D [D-Orn⁹,D-neo-Trp¹¹,tert-Leu¹²]NT(8-13), NT71D [N-methyl-Arg⁸,DAB⁹,D-neo-Trp¹¹,tert-Leu¹²]NT(8-13), NT78L [N-methyl-Arg⁸,D-Orn⁹ L-neo-Trp¹¹,tert-Leu¹²]NT(8-13), NT72L [D-Lys⁹,L-neo-Trp¹¹,tert-Leu¹²]NT(9-13), and NT78D [N-methyl-Arg⁸,D-Orn⁹,D-neo-Trp¹¹,tert-Leu¹²]NT(8-13), where neo-Trp is (2-amino-3-[1H-indolyl]propanoic acid). Other neurotensin analogs include β-lactotensin (NTR2 selective), JMV-449, and PD-149 or PD-163 (NTR1 selective; reduced amide bond 8-13 fragment of neurotensin).

Other neurotensin analogs include those with modified amino acids (e.g., any of those described herein). The neurotensin analog may be selective for NTR1, NTR2, or NTR3 (e.g., may bind to or activate one of NTR1, NTR2, or NTR3 at least 2, 5, 10, 50, 100, 500, 1000, 5000, 10,000, 50,000, or 100,000 greater) as compared to at least one of the other NTR receptors or both.

GDNF and GDNF Analogs

In certain embodiments, therapeutic agent is GDNF, a GDNF analog, a GDNF fragment, or a modified form thereof. In certain embodiments, the GDNF analog is a sequence substantially identical (e.g., at least 60%, 70%, 80%, 85%, 90%, 95%, 98%, 99% identical) to GDNF, a GDNF analog, or to a fragment thereof.

GDNF is secreted as a disulfide-linked homodimer, and is able to support survival of dopaminergic neurons, Purkinje cells, motoneurons, and sympathetic neurons. GDNF analogs or fragments having one or more of these activities may be used in the present invention, and activity of such analogs and fragments can be tested using any means known in the art.

Human GDNF is expressed as a 211 amino acid protein (isoform 1); a 185 amino acid protein (isoform 2), and a 133 amino acid protein. Mature GDNF is a 134 amino acid sequence that includes amino acids 118-211 of isoform 1, amino acids 92-185 of isoform 2. Isoform 3 includes a transforming growth factor like domain from amino acids 40-133.

In certain embodiments, the GDNF analog is a splice variant of GDNF. Such proteins are described in PCT Publication No. WO 2009/053536, and include the pre-(α) pro-GDNF, pre-(β)pro-GDNF, and pre-(γ)pro-GDNF splice variant, as well as the variants lacking the pre-pro region: (α) pro-GDNF, (β)pro-GDNF, and pre-(γ)pro-GDNF.

GDNF analogs are also described in U.S. Patent Application Publication No. 2009/0069230, which include a GDNF analog having the sequence:

Xaa₁-Pro-Xaa₃-Pro-Xaa₅-Xaa₆-Xaa₇-Xaa₈.

where Xaa₁ is Phe, Trp, or Tyr; Xaa₃ is Leu, Ala, Ile, or Val; Xaa₅ is Ala, Leu, Ile, or Val; Xaa₆ is Gly, is any amino acid residue of the D configuration or is absent; Xaa₇ is Lys, Arg, or His or is absent; and Xaa₈ is Arg, Lys, or His or is absent. Xaa represents an amino acid, which we may also refer to as an amino acid residue. The subscripts (here, the subscripts 1-8) represent the positions of each amino acid in the peptide sequence. Thus, Xaa₁ represents the first amino acid residue in a fragment of a GDNF precursor protein.

In specific embodiments, the fragments of a GDNF precursor protein can have a sequence represented by (1) Phe-Pro-Xaa₃-Pro-Xaa₅-Xaa₆-Xaa₇-Xaa₈, (e.g., Phe-Pro-Leu-Pro-Ala-Gly-Lys-Arg); (2) Xaa₁-Pro-Leu-Pro-Xaa₅-Xaa₆-Xaa₇-Xaa₈; (3) Phe-Pro-Leu-Pro-Xaa₅-Xaa₆-Xaa₇-Xaa₈; (4) Xaa₁-Pro-Xaa₃-Pro-Ala-Xaa₆-Xaa₇-Xaa₈; (5) Phe-Pro-Xaa₃-Pro-Ala-Xaa₆-Xaa₇-Xaa₈; (6) Phe-Pro-Leu-Pro-Ala-Xaa₆-Xaa₇-Xaa₈; (7) Xaa₁-Pro-Xaa₃-Pro-Xaa₅-Gly-Xaa₇-Xaa₈; (8) Phe-Pro-Xaa₃-Pro-Xaa₅-Gly-Xaa₇-Xaa₈; (9) Phe-Pro-Leu-Pro-Xaa₅-Gly-Xaa₇-Xaa₈; (10) Phe-Pro-Leu-Pro-Ala-Gly-Xaa₇-Xaa₈; (11) Xaa₁-Pro-Xaa₃-Pro-Xaa₅-Xaa₆-Lys-Xaa₈; (12) Phe-Pro-Xaa₃-Pro-Xaa₅-Xaa₆-Lys-Xaa₈; (13) Phe-Pro-Leu-Pro-Xaa₅-Xaa₆-Lys-Xaa₈; (14) Phe-Pro-Leu-Pro-Ala-Xaa₆-Lys-Xaa₈; (15) Phe-Pro-Leu-Pro-Ala-Gly-Lys-Xaa₈; (16) Xaa₁-Pro-Xaa₃-Pro-Xaa₅-Xaa₆-Xaa₇-Arg; (17) Phe-Pro-Xaa₃-Pro-Xaa₅-Xaa₆-Xaa₇-Arg; (18) Phe-Pro-Leu-Pro-Xaa₅-Xaa₆-Xaa₇-Arg; (19) Phe-Pro-Leu-Pro-Ala-Xaa₆-Xaa₇-Arg; and (20) Phe-Pro-Leu-Pro-Ala-Gly-Xaa₇-Arg.

In another embodiment, the fragment of a GDNF precursor protein can be a fragment or portion of a GDNF precursor protein conforming to Formula I, where Xaa₁ is Phe, Xaa₃ is Leu, Xaa₅ is Ala, Xaa₆ is Gly, Xaa₇ is Lys and Xaa₈ is Arg (i.e., Phe-Pro-Leu-Pro-Ala-Gly-Lys-Arg). At least one (e.g., one, two, or three) of the amino acid residues represented by Formula I can be absent. For example, Xaa₆, Xaa₇, and/or Xaa₈ can be absent.

In another embodiment, the fragment of a GDNF precursor protein or the biologically active variants can have, or can include, a sequence of amino acid residues conforming to the amino acid sequence:

Pro-Pro-Xaa₃-Xaa₄-Pro-Xaa₆-Xaa₇-Xaa₈-Xaa₉-Xa- a₁₀- Xaa₁₁-Xaa₁₂-Xaa₁₃-Xaa₁₄

where Xaa₃ is Glu or Asp; Xaa₄ is Ala, Gly, Ile, Leu, Met, or Val; Xaa₆ is Ala, Gly, Ile, Leu, Met, or Val; Xaa₇ is Glu or Asp; Xaa₈ is Asp or Glu; Xaa₉ is Arg, His, or Lys; Xaa₁₀ is Ser, Asn, Gln, or Thr; Xaa₁₁ is Leu, Ala, Gly, Ile, Leu, Met or Val; Xaa₁₂ is Gly, is any amino acid residue of the D-configuration, or is not present; Xaa₁₃ is Arg, His, or Lys or is not present; Xaa₁₄ is Mg, His, or Lys or is not present. An exemplary peptide conforming to Formula II can have the sequence Pro-Pro-Glu-Ala-Pro-Ala-Glu-Asp-Arg-Ser-Leu-Gly-Arg-Arg.

In another embodiment, the fragments of a GDNF precursor protein or the biologically active variants can have, or can include, a sequence of amino acid residues conforming to the amino acid sequence of Formula III:

X₁-X₂-X₃-X₄-X₅-X₆-X₇-X₈-X₉- X₁₀-X₁₁-X₁₂-X₁₃-X₁₄-X₁₅-X₁₆-X₁₇-X₁₈-X₁₉-X₂₀-X₂₁-X₂₂ (III).

where X₁ and X₂ are, independently, Mg, Lys, or His or are absent; X₃ is Glu or Asp; X₄ is Arg, Lys, or His; X₅ is Asn, Gln, Ser, or Thr; X₆ is Arg, Lys, or His; X₇ is Gln, Asn, Ser, or Thr; X₈, X₉, X₁₀, and X₁₁ are, independently, Ala, Gly, Ile, Leu, Met, or Val; X₁₂ is Asn, Gln, Ser, or Thr; X₁₃ is Pro or Ser; X₁₄ is Glu or Asp; X₁₅ is Asn, Gln, Ser, or Thr; X₁₆ is Ser, Asn, Gln, or Thr; X₁₇ is Lys, Arg, or His; X₁₈ is Gly, Ala, Ile, Leu, Met, or Val; X₁₉ is Lys, Arg, or His; X₂₀ is Gly, is any amino acid residue of the D-configuration, or is not present; and X₂₁ and X₂₂ are, independently, Arg, Lys, His, or are not present. An exemplary peptide conforming to Formula III can have the sequence Arg-Arg-Glu-Arg-Asn-Arg-Gln-Ala-Ala-Ala-Ala-Asn-Pro-Glu-Asn-Ser-Arg-Gly-Lys-Gly-Arg-Arg.

Other GDNF analogs are described in PCT Publication No. WO 2008/069876. These analogs include ERNRQAAAANPENSRGK-amide; FPLPA-amide; and PPEAPAEDRSL-amide.

Still other GDNF analogs are described in PCT Publication No. WO 2007/019860. The analogs include those having the formula:

Xₐ-(x)-X_{b}-X_{c}-X_{d}-X_{f}

where Xₐ is D, E, A or G, (x) is a sequence of 2-3 amino acid residues or a single amino acid residue selected from the group consisting of amino acid residues A, D, E, G, I, K, L, P, Q, S, T and V, X_{b} is amino acid residue Y or H, or a hydrophobic amino acid residue, and at least one of X_{c}, X_{d}, or X_{f} is a charged or hydrophobic amino acid residue. The analog may be 6-22 amino acids in length.

Further GDNF analogs are described in U.S. Patent Application Publication No. 2006/0258576. These analogs include FPLPA-amide, PPEAPAEDRSL-amide, LLEAPAEDHSL-amide, SPDKQMAVLP, SPDKQAAALP, SPDKQTPIFS, ERNRQAAAANPENSRGK-amide, ERNRQAAAASPENSRGK-amide, and ERNRQSAATNVENSSKK-amide.

Additional GDNF analogs can include functional fragments (e.g., any of the fragments described herein), peptides having any of the modifications described herein, or peptidomimetics thereof. Activity of such analogs and fragments can be tested using any means known in the art.

Brain-Derived Neurotrophic Factor (BDNF) and BDNF Analogs

The compounds of the invention may be or may include BDNF, BNDF analogs, or BNDF fragments. BDNF is glycoprotein of the nerve growth factor family of proteins. The protein is encoded as a 247 amino acid polypeptide (isoform A), a 255 amino acid polypeptide (isoform B), a 262 amino acid polypeptide (isoform C), a 276 amino acid polypeptide (isoform D), a 329 amino acid polypeptide (isoform E). The mature 119 amino acid glycoprotein is processed from the larger precursor to yield a neutrophic factor that promotes the survival of neuronal cell populations. The mature protein includes amino acids 129-247 of the isoform A preprotein, amino acids 137-255 of the isoform B preprotein, amino acids 144-162 of isoform C preprotein, amino acids 158-276 of the isoform D preprotein, or amino acids 211 (or 212)-329 of the isoform E preprotein. BDNF acts at the TrkB receptor and at low affinity nerve growth factor receptor (LNGFR or p75). BDNF is capable of supporting neuronal survival of existing neurons and can also promote growth and differentiation of new neurons. The BDNF fragments or analogs of the invention may have any of the aforementioned activities. Activity of such analogs and fragments can be tested using any means known in the art.

BDNF analogs are described in U.S. Patent Application Publication No. 2004/0072291, which include those having a substitution of A, C, D, E, G, H, K, N P, Q R, S, or Tat one more positions selected from the group consisting of 10, 16, 20, 29, 31, 36, 38, 39, 42, 44, 49, 52, 53, 54, 61, 63, 71, 76, 86, 87, 90, 92, 98, 100, 102, 103, and 105. Additional substitutions are described in Table 4 below.

TABLE 4

| Resi- | | |
|---|---|---|
| due | WT | |
| # | Residue | Possible substitutions |
| 9 | E | A C F G I L M P V W Y |
| 10 | L | I M F V W Y |
| 11 | S | A C F G I L M P V W Y |
| 13 | C | D E F H I K N P Q R S T V Y |
| 14 | D | A C F G I L M P V W Y |
| 15 | S | D F H I L N P Q W Y |
| 16 | I | W M Y |
| 17 | S | A C G P |
| 18 | E | T F H I P Q S |
| 19 | W | A C D E G H K N P Q R S T |
| 20 | V | W Y |
| 21 | T | D F H I L P W Y |
| 22 | A | D E H K N P Q R S T |
| 23 | A | H T |
| 24 | D | H P T |
| 28 | A | H T |
| 31 | M | W Y |
| 32 | S | A C G P |
| 34 | G | T D E H K N P Q R S |
| 35 | T | A C G P |
| 36 | V | F I L M W Y |
| 38 | V | W Y F I M |
| 39 | L | F I M V W Y |
| 41 | K | A C G H P S |
| 42 | V | I |
| 44 | V | F L M W Y |
| 45 | S | A C F P V Y |
| 46 | K | A C G P Q S T |
| 47 | G | D E H N P Q R S T |
| 48 | Q | A C G P |
| 49 | L | F I M V W Y |
| 50 | K | I P T |
| 51 | Q | A C G P |
| 52 | Y | I M V W |
| 53 | F | M W Y |
| 55 | E | A C G H N P Q S T |
| 56 | T | A C G P |
| 57 | K | A C G H P Q S T |
| 58 | C | D E G H K N P Q R S T |
| 59 | N | A C G P T |
| 60 | P | T |
| 61 | M | I V W Y |
| 87 | V | F I M W Y |
| 88 | R | A C G P |
| 89 | A | D E H K N Q R T |
| 90 | L | F I M V W Y |
| 91 | T | A C P G P |
| 92 | H | I W Y |
| 93 | D | P T |
| 94 | S | A C G P |
| 95 | K | H P |
| 96 | K | P |
| 97 | R | A C G P |
| 98 | I | H W |
| 101 | R | P T |
| 102 | F | I M V W Y |
| 103 | I | F M W Y |
| 104 | R | A C G P T |
| 105 | I | M W |
| 106 | D | A C G H I M P T |
| 107 | T | A C D E G H K N P Q S |
| 108 | S | A C D G H P |
| 109 | C | D E H K N P Q R S T |
| 110 | V | T |
| 111 | C | D E F H I K N P Q R S T V W Y |
| 112 | T | A C F G I L H P V W Y |
| 113 | L | Any amino acid |

BDNF analogs are also described in U.S. Pat. No. 6,800,607, which describes BDNF modified with 1-acyl-glycerol. These analogs include (1) a BDNF modified with a 1-acyl-glycerol derivative; (2) a modified BDNF, where is the compound of the formula (I):

A(X-B)ₙ

where A is a residue of brain-derived neurotrophic factor, B is a residue of a 1-acyl-glycerol derivative having a hydroxyl group at the 2-position of the glycerol moiety, which is prepared by removing a hydrogen atom from the hydroxyl group, X is a chemical cross-linkage, and m is an average number of the introduction and is not less than about 0.5; (3) a modified BDNF according to the above (2), wherein X is a group of the formula (II):

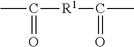

where R¹ is an alkylene group, or a group of the formula (III):

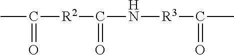

where R² and R³ are independently an alkylene group; (4) a modified BDNF according to the above (2), wherein the 1-acyl-glycerol derivative is 1-acyl-glycero-3-phosphoryl choline, 1-acyl-glycero-3-phosphoryl serine, or 1-acyl-grycero-3-phosphoryl ethylamine; (5) a modified BDNF according to the above (2), wherein B is a 1-acyl-glycero-3-phosphoryl choline residue of the formula (IV):

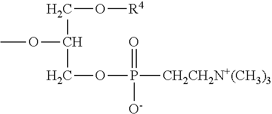

where R⁴ is an acyl group, a 1-acyl-glycero-3-phosphoryl serine residue of the formula (V):

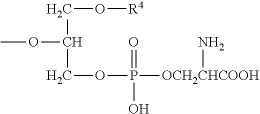

where R⁴ is an acyl group, or a 1-acyl-glycero-phosphoryl ethylamine residue of the formula (VI):

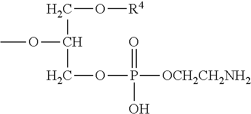

where R⁴ is an acyl group; (6) a modified BDNF according to the above (2) or (3), where B is a group of the formula (IV):

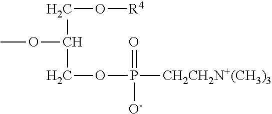

where R⁴ is an acyl group; (7) a modified BDNF according to any one of the above (2), (3), (4), (5) and (6), where the acyl group is an alkanoyl group having 8 to 30 carbon atoms; (8) a modified BDNF according to any one of the above (2), (3), (4), (5), (6) and (7), where the acyl group is palmitoyl group; (9) a modified BDNF according to any one of the above (2), (3), (4), (5), (6), (7) and (8), where m is in the range of from about 1 to about 6; (10) a modified BDNF according to any one of the above (2), (3), (4), (5), (6), (7), (8) and (9), wherein X is a group of the formula (II):

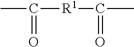

where R¹ is an alkylene group; (11) a modified BDNF according to the above (10), where R¹ is a straight chain alkylene group having 2 to 10 carbon atoms; and (12) a modified BDNF according to the above (10), where R¹ is trimethylene.

Other BDNF analogs include those described in PCT Publication No. WO 96/15146, which describes conjugates of BDNF to water soluble polymers such as polyethylene glycol. Additional BDNF analogs can include functional fragments (e.g., any of the fragments described herein), peptides having any of the modifications described herein, or peptidomimetics thereof. Activity of such analogs can be tested using any method known in the art.

Hydrophobic Agents

Any hydrophobic agent may be used in the compositions and methods of the present invention. Nanoparticle and micelle-based delivery methods that use amphipathic molecules are especially well suited for delivery of hydrophobic agents (e.g., any agent that exhibits low solubility in aqueous solution. Exemplary hydrophobic agents are described below and include analgesics and antiinflammatory agents (e.g., aloxiprin, auranofin, azapropazone, benorylate, diflunisal, etodolac, fenbufen, fenoprofen calcim, flurbiprofen, ibuprofen, indomethacin, ketoprofen, meclofenamic acid, mefenamic acid, nabumetone, naproxen, oxyphenbutazone, phenylbutazone, piroxicam, sulindac), antihelmintics (e.g., albendazole, bephenium hydroxynaphthoate, cambendazole, dichlorophen, ivermectin, mebendazole, oxamniquine, oxfendazole, oxantel embonate, praziquantel, pyrantel embonate, thiabendazole), anti-arrhythmic agents (e.g., amiodarone (e.g., HCl), disopyramide, flecamide (e.g., acetate), quinidine (e.g., sulfate)), anti-bacterial agents (e.g., benethamine penicillin, cinoxacin, ciprofloxacin (e.g., HCl), clarithromycin, clofazimine, cloxacillin, demeclocycline, doxycycline, erythromycin, ethionamide, imipenem, nalidixic acid, nitrofurantoin, rifampicin, spiramycin, sulphabenzamide, sulphadoxine, sulphamerazine, sulphacetamide, sulphadiazine, sulphafurazole, sulphamethoxazole, sulphapyridine, tetracycline, trimethoprim), anti-coagulants (e.g., dicoumarol, dipyridamole, nicoumalone, phenindione), antidepressants (e.g., amoxapine, maprotiline (e.g., HCl), mianserin (e.g., HCl), nortriptyline (e.g., HCl), trazodone (e.g., HCl), trimipramine (e.g., maleate)), antidiabetics (e.g., acetohexamide, chlorpropamide, glibenclamide, gliclazide, glipizide, tolazamide, tolbutamide), anti-epileptics (e.g., beclamide, carbamazepine, clonazepam, ethotoin, methoin, methsuximide, methylphenobarbitone, oxcarbazepine, paramethadione, phenacemide, phenobarbitone, phenyloin, phensuximide, primidone, sulthiame, valproic acid), antifungal agents (e.g., amphotericin, butoconazole (e.g., nitrate), clotrimazole, econazole (e.g., nitrate), fluconazole, flucytosine, griseofulvin, itraconazole, ketoconazole, miconazole, natamycin, nystatin, sulconazole (e.g., nitrate), terbinafine (e.g., HCl), terconazole, tioconazole, undecenoic acid), antigout agents (e.g., allopurinol, probenecid, sulphin-pyrazone), antihypertensive agents (e.g., amlodipine, benidipine, darodipine, dilitazem (e.g., HCl), diazoxide, felodipine, guanabenz (e.g., acetate), isradipine, minoxidil, nicardipine (e.g., HCl), nifedipine, nimodipine, phenoxybenzamine (e.g., HCl), prazosin (e.g., HCl), reserpine, terazosin (e.g., HCl)), antimalarials (e.g., amodiaquine, chloroquine, chlorproguanil (e.g., HCl), halofantrine (e.g., HCl), mefloquine (e.g., HCl), proguanil (e.g., HCl), pyrimethamine, quinine sulphate), anti-migraine agents (e.g., dihydroergotamine (e.g., mesylate), ergotamine (e.g., tartrate), methysergide (e.g., maleate), pizotifen (e.g., maleate), sumatriptan succinate), anti-muscarinic agents (e.g., atropine, benzhexyl (e.g., HCl), biperiden, ethopropazine (e.g., HCl), hyoscyamine, mepenzolate (e.g., bromide), oxyphencylcimine (e.g., HCl), tropicamide), anticancer agents and immunosuppressants (e.g., aminoglutethimide, amsacrine, azathioprine, busulphan, chlorambucil, cyclosporin, dacarbazine, doxorubicin, estramustine, etoposide, lomustine, melphalan, mercaptopurine, methotrexate, mitomycin, mitotane, mitozantrone, paclitaxel, procarbazine (e.g., HCl), tamoxifen (e.g., citrate), testolactone), anti-protazoal agents (e.g., benznidazole, clioquinol, decoquinate, diiodohydroxyquinoline, diloxanide furoate, dinitolmide, furzolidone, metronidazole, nimorazole, nitrofurazone, ornidazole, timidazole), anti-thyroid agents (e.g., carbimazole, propylthiouracil), anxiolytic, sedatives, hypnotics and neuroleptics (e.g., alprazolam, amylobarbitone, barbitone, bentazepam, bromazepam, bromperidol, brotizolam, butobarbitone, carbromal, chlordiazepoxide, chlormethiazole, chlorpromazine, clobazam, clotiazepam, clozapine, diazepam, droperidol, ethinamate, flunanisone, flunitrazepam, fluopromazine, flupenthixol decanoate, fluphenazine decanoate, flurazepam, haloperidol, lorazepam, lormetazepam, medazepam, meprobamate, methaqualone, midazolam, nitrazepam, oxazepam, pentobarbitone, perphenazine pimozide, prochlorperazine, sulpiride, temazepam, thioridazine, triazolam, zopiclone), (3-Blockers (e.g., acebutolol, alprenolol, atenolol, labetalol, metoprolol, nadolol, oxprenolol, pindolol, propranolol), cardiac inotropic agents (e.g., aminone, digitoxin, digoxin, enoximone, lanatoside C, medigoxin), corticosteroids (e.g., beclomethasone, betamethasone, budesonide, cortisone (e.g., acetate), desoxymethasone, dexamethasone, fludrocortisone (e.g., acetate), flunisolide, flucortolone, fluticasone (e.g., propionate), hydrocortisone, methylprednisolone, prednisolone, prednisone, triamcinolone), diuretics (e.g., acetazolamide, amiloride, bendrofluazide, bumetanide, chlorothiazide, chlorthalidone, ethacrynic acid, frusemide, metolazone, spironolactone, triamterene), anti-parkinsonian agents (e.g., bromocriptine (e.g., mesylate), lysuride (e.g., maleate)), gastrointestinal agents (e.g., bisacodyl, cimetidine, cisapride, diphenoxylate (e.g., HCl), domperidone, famotidine, loperamide, mesalazine, nizatidine, omeprazole, ondansetron (e.g., HCl), ranitidine (e.g., HCl), sulphasalazine), histamine H-receptor antagonists (e.g., acrivastine, astemizole, cinnarizine, cyclizine, cyproheptadine (e.g., HCl), dimenhydrinate, flunarizine (e.g., HCl), loratadine, meclozine (e.g., HCl), oxatomide, terfenadine), lipid regulating agents (e.g., bezafibrate, clofibrate, fenofibrate, gemfibrozil, probucol), nitrates and other anti-anginal agents (e.g., amyl nitrate, glyceryl trinitrate, isosorbide dinitrate, isosorbide mononitrate, pentaerythritol tetranitrate), opioid analgesics (e.g., codeine, dextropropyoxyphene, diamorphine, dihydrocodeine, meptazinol, methadone, morphine, nalbuphine, pentazocine), sex hormones (e.g., clomiphene (e.g., citrate), danazol, ethinyl estradiol, medroxyprogesterone (e.g., acetate), mestranol, methyltestosterone, norethisterone, norgestrel, estradiol, conjugated oestrogens, progesterone, stanozolol, stibestrol, testosterone, tibolone), and stimulants (e.g., amphetamine, dexamphetamine, dexfenfluramine, fenfluramine, mazindol). The invention may also include analogs of any of these agents (e.g., therapeutically effective analogs).

### Therapeutic Indications

The conjugates of the invention can be used to treat any disease or condition that the agent contained within the vector may be used to treat. Exemplary disease and conditions are described below.

Cancer

The conjugates and compositions of the invention can be used to treat any cancer, but, in the case of conjugates including a vector that is efficiently transported across the BBB, are particularly useful for the treatment of brain cancers and other cancers protected by the BBB. These cancers include astrocytoma, pilocytic astrocytoma, dysembryoplastic neuroepithelial tumor, oligodendrogliomas, ependymoma, glioblastoma multiforme, glioma, neuroglioma, mixed gliomas, oligoastrocytomas, hemangioma, medulloblastoma, retinoblastoma, neuroblastoma, germinoma, teratoma, and meningioma.

Metastatic cancer can originate from cancer of any tissue, including any described herein. Exemplary metastatic cancers include those originating from brain cancer, breast cancer, colon cancer, prostate cancer, ovarian cancer, sarcoma, bladder cancer, neuroblastoma, Wilm's tumor, lymphoma, non-Hodgkin's lymphoma, and certain T-cell lymphomas.

Other types of cancer include hepatocellular carcinoma, breast cancer, cancers of the head and neck including various lymphomas such as mantle cell lymphoma, non-Hodgkins lymphoma, adenoma, squamous cell carcinoma, laryngeal carcinoma, cancers of the retina, cancers of the esophagus, multiple myeloma, ovarian cancer, uterine cancer, melanoma, colorectal cancer, bladder cancer, prostate cancer, lung cancer (including non-small cell lung carcinoma), pancreatic cancer, cervical cancer, head and neck cancer, skin cancers, nasopharyngeal carcinoma, liposarcoma, epithelial carcinoma, renal cell carcinoma, gallbladder adenocarcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease.

Neurodegenerative Disease

Because polypeptides described herein are capable of transporting an agent across the BBB, the conjugates of the invention are also useful for the treatment of neurodegenerative diseases or other conditions affecting the mammalian brain, central nervous system (CNS), the peripheral nervous system, or the autonomous nervous system wherein neurons are lost or deteriorate. Many neurodegenerative diseases are characterized by ataxia (i.e., uncoordinated muscle movements) and/or memory loss. Neurodegenerative diseases include Alexander disease, Alper disease, Alzheimer's disease, amyotrophic lateral sclerosis (ALS; i.e., Lou Gehrig's disease), ataxia telangiectasia, Batten disease (Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbé disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Schilder's disease (i.e., adrenoleukodystrophy), schizophrenia, spinocerebellar ataxia, spinal muscular atrophy, Steele-Richardson, Olszewski disease, and tabes dorsalis.

Lysosomal Storage Disorders

The conjugates and compositions of the invention may also be used to treat a lysosomal storage disease or disorder, many of which affect the central nervous system (CNS) and cause or exacerbate neurodegenerative disease. Lysosomal storage disorders are caused typically by a deficiency in a gene/protein and thus are amenable to treatment by administration of agent that is able to restore the deficiency. Lysosomal storage diseases include any of the mucopolysaccharidoses (MPS; including MPS-I (Hurler syndrome or Scheie syndrome), MPS-II (Hunter syndrome), MPS-IIIA (Sanfilippo syndrome A), MPS-IIIB (Sanfilippo syndrome B), MPS-IIIC (Sanfilippo syndrome C), MPS-IIID (Sanfilippo syndrome D), MPS-IV (Morquio syndrome), MPS-VI (Maroteaux-Lamy syndrome), MPS-VII (Sly syndrome), and MPS-IX (hyaluronidase deficiency)), lipidoses (including Gaucher' disease, Niemann-Pick disease, Fabry disease, Farber's disease, and Wolman's disease), gangliosidoses (including GM1 and GM2 gangliosidoses, Tay-Sachs disease, and Sandhoff disease), leukodystrophies (including adrenoleukodystrophy (i.e., Schilder's disease), Alexander disease, metachromatic leukodystrophy, Krabbé disease, Pelizaeus-Merzbacher disease, Canavan disease, childhood ataxia with central hypomyelination (CACH), Refsum's disease, and cerebrotendineous xanthomatosis), mucolipidoses (ML; including ML-I (sialidosis), ML-II (I-cell disease), ML-III (pseudo-Hurler polydystrophy), and ML-IV), and glycoproteinoses (including aspartylglucosaminuria, fucosidosis, and mannosidosis).

Therapeutic Applications for GLP-1 Agonists

The conjugates and compositions of the invention can be used in any therapeutic application where a GLP-1 agonist activity in the brain, or in particular tissues, is desired. GLP-1 agonist activity is associated with stimulation of insulin secretion (i.e., to act as an incretin hormone) and inhibition glucagon secretion, thereby contributing to limit postprandial glucose excursions. GLP-1 agonists can also inhibit gastrointestinal motility and secretion, thus acting as an enterogastrone and part of the “ileal brake” mechanism. GLP-1 also appears to be a physiological regulator of appetite and food intake. Because of these actions, GLP-1 and GLP-1 receptor agonists can be used for therapy of metabolic disorders, as reviewed in, e.g., Kinzig et al., J. Neurosci. 23:6163-6170, 2003. Such disorders include obesity, hyperglycemia, dyslipidemia, hypertriglyceridemia, syndrome X, insulin resistance, IGT, diabetic dyslipidemia, hyperlipidemia, a cardiovascular disease, and hypertension.

GLP-1 is also has neurological effects including sedative or anti-anxiolytic effects, as described in U.S. Pat. No. 5,846,937. Thus, GLP-1 agonists can be used in the treatment of anxiety, aggression, psychosis, seizures, panic attacks, hysteria, or sleep disorders. GLP-1 agonists can also be used to treat Alzheimer's disease, as GLP-1 agonists have been shown to protect neurons against amyloid-ii peptide and glutamate-induced apoptosis (Perry et al., Curr. Alzheimer. Res. 2:377-85, 2005).

Other therapeutic uses for GLP-1 agonists include improving learning, enhancing neuroprotection, and alleviating a symptom of a disease or disorder of the central nervous system, e.g., through modulation of neurogenesis, and, e.g., Parkinson's Disease, Alzheimer's Disease, Huntington's Disease, ALS, stroke, ADD, and neuropsychiatric syndromes (U.S. Pat. No. 6,969,702 and U.S. Patent Application Publication No. 2002/0115605). Stimulation of neurogenesis using GLP-1 agonists has been described, for example, in Bertilsson et al., J. Neurosci. Res. 86:326-338, 2008.

Still other therapeutic uses include converting liver stem/progenitor cells into functional pancreatic cells (U.S. Patent Application Publication No. 2005/0053588); preventing beta-cell deterioration (U.S. Pat. Nos. 7,259,233 and 6,569,832) and stimulation of beta-cell proliferation (U.S. Patent Application Publication No. 2003/0224983); treating obesity (U.S. Pat. No. 7,211,557); suppressing appetite and inducing satiety (U.S. Patent Application Publication No. 2003/0232754); treating irritable bowel syndrome (U.S. Pat. No. 6,348,447); reducing the morbidity and/or mortality associated with myocardial infarction (U.S. Pat. No. 6,747,006) and stroke (PCT Publication No. WO 00/16797); treating acute coronary syndrome characterized by an absence of Q-wave myocardial infarction (U.S. Pat. No. 7,056,887); attenuating post-surgical catabolic changes (U.S. Pat. No. 6,006,753); treating hibernating myocardium or diabetic cardiomyopathy (U.S. Pat. No. 6,894,024); suppressing plasma blood levels of norepinepherine (U.S. Pat. No. 6,894,024); increasing urinary sodium excretion, decreasing urinary potassium concentration (U.S. Pat. No. 6,703,359); treating conditions or disorders associated with toxic hypervolemia, e.g., renal failure, congestive heart failure, nephrotic syndrome, cirrhosis, pulmonary edema, and hypertension (U.S. Pat. No. 6,703,359); inducing an inotropic response and increasing cardiac contractility (U.S. Pat. No. 6,703,359); treating polycystic ovary syndrome (U.S. Pat. No. 7,105,489); treating respiratory distress (U.S. Patent Application Publication No. 2004/0235726); improving nutrition via a non-alimentary route, i.e., via intravenous, subcutaneous, intramuscular, peritoneal, or other injection or infusion (U.S. Pat. No. 6,852,690); treating nephropathy (U.S. Patent Application Publication No. 2004/0209803); treating left ventricular systolic dysfunction, e.g., with abnormal left ventricular ejection fraction (U.S. Pat. No. 7,192,922); inhibiting antro-duodenal motility, e.g., for the treatment or prevention of gastrointestinal disorders such as diarrhea, postoperative dumping syndrome and irritable bowel syndrome, and as premedication in endoscopic procedures (U.S. Pat. No. 6,579,851); treating critical illness polyneuropathy (CIPN) and systemic inflammatory response syndrome (SIRS) (U.S. Patent Application Publication No. 2003/0199445); modulating triglyceride levels and treating dyslipidemia (U.S. Patent Application Publication Nos. 2003/0036504 and 2003/0143183); treating organ tissue injury caused by reperfusion of blood flow following ischemia (U.S. Pat. No. 6,284,725); treating coronary heart disease risk factor (CHDRF) syndrome (U.S. Pat. No. 6,528,520); and others.

Therapeutic Applications for Leptin and Leptin Analogs

The conjugates and compositions of the invention can be used to treat a metabolic disorder, e.g., where the transport vector contains leptin or an analog thereof. Such disorders include diabetes (type I or type II), obesity, hyperglycemia, dyslipidemia, hypertriglyceridemia, syndrome X, insulin resistance, IGT, diabetic dyslipidemia, hyperlipidemia, a cardiovascular disease, and hypertension. Leptin decreases food intake and thus can be used to reduce weight and to treat diseases where reduced food intake or weight loss is beneficial.

Therapeutic Applications for NT and NT Analogs

Various therapeutic applications have been suggested for NT, including psychiatric disorders, metabolic disorder, and pain. Because neurotensin has been shown to modulate neurotransmission in areas of the brain associated with schizophrenia, neurotensin and neurotensin receptor agonists have been proposed as antipsychotic agents.

Neurological Disease

Because the vector conjugates and compositions of the invention can transport an agent across the BBB, the compounds of the invention are also useful for the treatment of neurological diseases such as neurodegenerative diseases or other conditions of the central nervous system (CNS), the peripheral nervous system, or the autonomous nervous system (e.g., where neurons are lost or deteriorate). NT has been suggested an antipsychotic therapy, and thus may be useful in the treatment of diseases such as schizophrenia and bipolar disorder. Many neurodegenerative diseases are characterized by ataxia (i.e., uncoordinated muscle movements) and/or memory loss. Neurodegenerative diseases include Alexander disease, Alper disease, Alzheimer's disease, amyotrophic lateral sclerosis (ALS; i.e., Lou Gehrig's disease), ataxia telangiectasia, Batten disease (Spielmeyer-Vogt-Sjogren-Batten disease), bovine spongiform encephalopathy (BSE), Canavan disease, Cockayne syndrome, corticobasal degeneration, Creutzfeldt-Jakob disease, Huntington's disease, HIV-associated dementia, Kennedy's disease, Krabbé disease, Lewy body dementia, Machado-Joseph disease (Spinocerebellar ataxia type 3), multiple sclerosis, multiple system atrophy, narcolepsy, neuroborreliosis, Parkinson's disease, Pelizaeus-Merzbacher disease, Pick's disease, primary lateral sclerosis, prion diseases, Refsum's disease, Schilder's disease (i.e., adrenoleukodystrophy), schizophrenia, spinocerebellar ataxia, spinal muscular atrophy, Steele-Richardson, Olszewski disease, and tabes dorsalis.

Inducing Body Temperature Reduction

The conjugates and compositions of the invention that include NT or an NT analog can be used to reduce the body temperature of a subject. Because reduction in body temperature has been shown to be beneficial in subjects who may be suffering from, or may have recently suffered from, a stroke, cerebral ischemia, cardiac ischemia, or a nerve injury such as a spinal chord injury, such a treatment would therefore be useful in reducing complications of these conditions.

Pain

NT is also known to have analgesic effects. Thus the conjugates and compositions of the invention that include NT or an NT analog may be used to reduce pain in a subject. The subject may be suffering from an acute pain (e.g., selected from the group consisting of mechanical pain, heat pain, cold pain, ischemic pain, and chemical-induced pain). Other types of pain include peripheral or central neuropathic pain, inflammatory pain, migraine-related pain, headache-related pain, irritable bowel syndrome-related pain, fibromyalgia-related pain, arthritic pain, skeletal pain, joint pain, gastrointestinal pain, muscle pain, angina pain, facial pain, pelvic pain, claudication, postoperative pain, post traumatic pain, tension-type headache, obstetric pain, gynecological pain, or chemotherapy-induced pain.

Metabolic Disorders

There is evidence that NT can be used to treat metabolic disorders; see, e.g., U.S. Patent Application Publication No. 2001/0046956. Thus the conjugates and compositions of the invention may be used to treat such disorders. The metabolic disorder may be diabetes (e.g., Type I or Type II), obesity, diabetes as a consequence of obesity, hyperglycemia, dyslipidemia, hypertriglyceridemia, syndrome X, insulin resistance, impaired glucose tolerance (IGT), diabetic dyslipidemia, hyperlipidemia, a cardiovascular disease, or hypertension. The subject may be overweight, obese, or bulimic.

Drug Addiction/Abuse

NT has also been suggested to be able to treat drug addiction or reduce drug abuse in subjects, particularly with psychostimulant. Thus the conjugates and compositions of the invention may be useful in treating addiction to or abuse of drugs such as amphetamine, methamphetamine, 3,4-methylenedioxymethamphetamine, nicotine, cocaine, methylphenidate, and arecoline.

Therapeutic Applications for GDNF, BDNF, and Analogs Thereof.

Any disease or condition where enhancing neuronal survival (e.g., decreasing neuronal death rate) or increasing the rate of neuronal formation is beneficial can be treated using the conjugates and compositions of the invention that includes GDNF, BDNF, or an analog thereof. Such conditions include neurodegenerative disorders, e.g., a disorder selected from the group consisting of a polyglutamine expansion disorder (e.g., Huntington's disease (HD), dentatorubropallidoluysian atrophy, Kennedy's disease (also referred to as spinobulbar muscular atrophy), and spinocerebellar ataxia (e.g., type 1, type 2, type 3 (also referred to as Machado-Joseph disease), type 6, type 7, and type 17)), another trinucleotide repeat expansion disorder (e.g., fragile X syndrome, fragile XE mental retardation, Friedreich's ataxia, myotonic dystrophy, spinocerebellar ataxia type 8, and spinocerebellar ataxia type 12), Alexander disease, Alper's disease, Alzheimer's disease, amyotrophic lateral sclerosis (ALS), ataxia telangiectasia, Batten disease (also referred to as Spielmeyer-Vogt-Sjogren-Batten disease), Canavan disease, Cockayne syndrome, corticobasal degeneration, Creutzfeldt-Jakob disease, ischemia stroke, Krabbe disease, Lewy body dementia, multiple sclerosis, multiple system atrophy, Parkinson's disease, Pelizaeus-Merzbacher disease, Pick's disease, primary lateral sclerosis, Refsum's disease, Sandhoff disease, Schilder's disease, spinal cord injury, spinal muscular atrophy, Steele-Richardson-Olszewski disease, and Tabes dorsalis. Other conditions include injury (e.g., spinal chord injury), concussion, ischemic stroke, and hemorrhagic stroke.

Additional Indications

The conjugates of the invention can also be used to treat diseases found in other organs or tissues. For example, Angiopep-7 (SEQ ID NO:112) is efficiently transported into liver, lung, kidney, spleen, and muscle cells, allowing for the preferential treatment of diseases associated with these tissues (e.g., hepatocellular carcinoma and lung cancer). The compositions and methods of the present invention may also be used to treat genetic disorders, such as Down syndrome (i.e., trisomy 21), where down-regulation of particular gene transcripts may be useful.

### Administration and Dosage

The present invention also relates pharmaceutical compositions that contain a therapeutically effective amount of a conjugate of the invention that is bound to or contains a therapeutic agent. The composition can be formulated for use in a variety of drug delivery systems. One or more physiologically acceptable excipients or carriers can also be included in the composition for proper formulation. Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990).

The pharmaceutical compositions are intended for parenteral, intranasal, topical, oral, or local administration, such as by a transdermal means, for prophylactic and/or therapeutic treatment. The pharmaceutical compositions can be administered parenterally (e.g., by intravenous, intramuscular, or subcutaneous injection), or by oral ingestion, or by topical application or intraarticular injection at areas affected by the vascular or cancer condition. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration. Sustained release administration is also specifically included in the invention, by such means as depot injections or erodible implants or components. Thus, the invention provides compositions for parenteral administration that comprise the above mention agents dissolved or suspended in an acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like. The invention also provides compositions for oral delivery, which may contain inert ingredients such as binders or fillers for the formulation of a tablet, a capsule, and the like. Furthermore, this invention provides compositions for local administration, which may contain inert ingredients such as solvents or emulsifiers for the formulation of a cream, an ointment, and the like.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The compositions containing an effective amount can be administered for prophylactic or therapeutic treatments. In prophylactic applications, compositions can be administered to a subject with a clinically determined predisposition or increased susceptibility to development of a tumor or cancer, neurodegenerative disease, or lysosomal disorder. Compositions of the invention can be administered to the patient (e.g., a human) in an amount sufficient to delay, reduce, or preferably prevent the onset of clinical disease or tumorigenesis. In therapeutic applications, compositions are administered to a subject (e.g., a human) already suffering from disease (e.g., a cancer, neurodegenerative disease, or lysosomal storage disorder) in an amount sufficient to cure or at least partially arrest the symptoms of the condition and its complications. An amount adequate to accomplish this purpose is defined as a “therapeutically effective dose,” an amount of a compound sufficient to substantially improve some symptom associated with a disease or a medical condition. For example, in the treatment of cancer, neurodegenerative disease, or lysosomal storage disease, an agent or compound which decreases, prevents, delays, suppresses, or arrests any symptom of the disease or condition would be therapeutically effective. A therapeutically effective amount of an agent or compound is not required to cure a disease or condition but will provide a treatment for a disease or condition such that the onset of the disease or condition is delayed, hindered, or prevented, or the disease or condition symptoms are ameliorated, or the term of the disease or condition is changed or, for example, is less severe or recovery is accelerated in an individual. Amounts effective for this use may depend on the severity of the disease or condition and the weight and general state of the patient, but generally range from about 0.5 mg to about 3000 mg of the agent or agents per dose per patient. Suitable regimes for initial administration and booster administrations are typified by an initial administration followed by repeated doses at one or more hourly, daily, weekly, or monthly intervals by a subsequent administration. The total effective amount of an agent present in the compositions of the invention can be administered to a mammal as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month). Alternatively, continuous intravenous infusion sufficient to maintain therapeutically effective concentrations in the blood are contemplated.

The therapeutically effective amount of one or more agents present within the compositions of the invention and used in the methods of this invention applied to mammals (e.g., humans) can be determined by the ordinarily-skilled artisan with consideration of individual differences in age, weight, and the condition of the mammal. The agents of the invention are administered to a subject (e.g. a mammal, such as a human) in an effective amount, which is an amount that produces a desirable result in a treated subject (e.g. the slowing or remission of a cancer or neurodegenerative disorder). Therapeutically effective amounts can be determined empirically by those of skill in the art.

The patient may also receive an agent in the range of about 0.1 to 3,000 mg per dose one or more times per week (e.g., 2, 3, 4, 5, 6, or 7 or more times per week), 0.1 to 2,500 (e.g., 2,000, 1,500, 1,000, 500, 100, 10, 1, 0.5, or 0.1) mg dose per week. A patient may also receive an agent of the composition in the range of 0.1 to 3,000 mg per dose once every two or three weeks.

Single or multiple administrations of the compositions of the invention comprising an effective amount can be carried out with dose levels and pattern being selected by the treating physician. The dose and administration schedule can be determined and adjusted based on the severity of the disease or condition in the patient, which may be monitored throughout the course of treatment according to the methods commonly practiced by clinicians or those described herein.

The carrier and conjugates of the present invention may be used in combination with either conventional methods of treatment or therapy or may be used separately from conventional methods of treatment or therapy.

When the conjugates of this invention are administered in combination therapies with other agents, they may be administered sequentially or concurrently to an individual. Alternatively, pharmaceutical compositions according to the present invention may be comprised of a combination of a carrier-agent conjugate of the present invention in association with a pharmaceutically acceptable excipient, as described herein, and another therapeutic or prophylactic agent known in the art.

### Further Conjugation

In the compositions and methods of the invention, the polypeptide-transport vector conjugate may be further linked to another agent, such as a therapeutic agent, a detectable label, or any other agent described herein. The conjugate may be labeled with a detectable label such as a radioimaging agent, such as those emitting radiation, for detection of a disease or condition. In other embodiments, the carrier or functional derivative thereof of the present invention or mixtures thereof may be linked to a therapeutic agent, to treat a disease or condition, or may be linked to or labeled with mixtures thereof. Treatment may be effected by administering a conjugate of the present invention that has been further conjugated to a therapeutic compound to an individual under conditions which allow transport of the agent across the BBB or to other cells or tissues where such treatment is beneficial.

A therapeutic agent as used herein may be a drug, a medicine, an agent emitting radiation, a cellular toxin (for example, a chemotherapeutic agent) and/or biologically active fragment thereof, and/or mixtures thereof to allow cell killing or it may be an agent to treat, cure, alleviate, improve, diminish or inhibit a disease or condition in an individual treated. A therapeutic agent may be a synthetic product or a product of fungal, bacterial or other microorganism, such as mycoplasma, viral etc., animal, such as reptile, or plant origin. A therapeutic agent and/or biologically active fragment thereof may be an enzymatically active agent and/or fragment thereof, or may act by inhibiting or blocking an important and/or essential cellular pathway or by competing with an important and/or essential naturally occurring cellular component.

Examples of radioimaging agents emitting radiation (detectable radio-labels) that may be suitable are exemplified by indium-111, technetium-99, or low dose iodine-131. Detectable labels, or markers, for use in the present invention may be a radiolabel, a fluorescent label, a nuclear magnetic resonance active label, a luminescent label, a chromophore label, a positron emitting isotope for PET scanner, chemiluminescence label, or an enzymatic label. Fluorescent labels include but are not limited to, green fluorescent protein (GFP), fluorescein, and rhodamine. Chemiluminescence labels include but are not limited to, luciferase and β-galactosidase. Enzymatic labels include but are not limited to peroxidase and phosphatase. A histamine tag may also be a detectable label. For example, conjugates may comprise a carrier moiety and an antibody moiety (antibody or antibody fragment) and may further comprise a label. The label may be for example a medical isotope, such as for example and without limitation, technetium-99, iodine-123 and -131, thallium-201, gallium-67, fluorine-18, indium-111, etc.

An agent may be releasable from the compound, conjugate, or composition after transport across the BBB, for example, by enzymatic cleavage or breakage of a chemical bond between the vector and the agent. The released agent may then function in its intended capacity in the absence of the vector.

Covalent modifications of the compounds, conjugates, and compositions of the invention are included within the scope of this invention. A chemical derivative may be conveniently prepared by direct chemical synthesis, using methods well known in the art. Such modifications may be, for example, introduced into a polypeptide, agent, or polypeptide-agent conjugate by reacting targeted amino acid residues with an organic derivatizing agent that is capable of reacting with selected side chains or terminal residues. A vector chemical derivative may be able, e.g., to cross the BBB and be attached to or conjugated with another agent, thereby transporting the agent across the BBB. The conjugate of the invention may be joined (i.e., conjugated) without limitation, through sulfhydryl groups, amino groups (amines) and/or carbohydrates to suitable detectable labels or therapeutic agents. Homobifunctional and heterobifunctional cross-linkers (conjugation agents) are available from many commercial sources. Regions available for cross-linking may be found on the carriers of the present invention. The cross-linker may comprise a flexible arm, such as for example, a short arm (<2 carbon chain), a medium-size arm (from 2-5 carbon chain), or a long arm (3-6 carbon chain). Exemplary cross-linkers include BS3 ([Bis(sulfosuccinimidyl)suberate]; BS3 is a homobifunctional N-hydroxysuccinimide ester that targets accessible primary amines), NHS/EDC(N-hydroxysuccinimide and N-ethyl-‘(dimethylaminopropyl)carbodimide; NHS/EDC allows for the conjugation of primary amine groups with carboxyl groups), sulfo-EMCS ([N-e-Maleimidocaproic acid]hydrazide; sulfo-EMCS are heterobifunctional reactive groups (maleimide and NHS-ester) that are reactive toward sulfhydryl and amino groups), hydrazide (most proteins contain exposed carbohydrates and hydrazide is a useful reagent for linking carboxyl groups to primary amines), and SATA (N-succinimidyl-5-acetylthioacetate; SATA is reactive towards amines and adds protected sulthydryls groups).

### Other Embodiments

All publications, patent applications, including U.S. Provisional Patent Application No. 61/249,152, filed Oct. 6, 2009, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, pharmacology, or related fields are intended to be within the scope of the invention.

What is claimed is:

1. A compound comprising:
(A) a polypeptide and a transport vector, wherein said polypeptide:
(a) comprises an amino acid sequence having at least 70% sequence identity to the sequence of SEQ ID NO:97 or to any of the sequences set forth in SEQ ID NOS:1-93, 98-105, and 107-116; and
(b) is conjugated to said transport vector, or
(B) the formula: A-X-B, wherein:
(a) A is a polypeptide comprising an amino acid sequence having at least 70% sequence identity to the sequence of Angiopep-2 (SEQ ID NO:97) or of SEQ ID NOS:1-93, 98-105, and 107-116;
(b) X is a linker; and
(c) B is a transport vector.

2. (canceled)

3. The compound of claim 1, wherein said amino acid sequence identity is at least 90%.

4. (canceled)

5. The compound of claim 1, wherein said polypeptide comprises the amino acid sequence set forth in one of SEQ ID NOS:67, 97, 107, 108, 109, 111, or 112.

6. The compound of claim 1, wherein said polypeptide or said compound is able to cross the blood-brain barrier in a mammal.

7. The compound of claim 1, wherein said polypeptide is 10 to 50 amino acid residues in length.

8. The compound of claim 1, wherein said transport vector is a lipid vector, a nanoparticle, a polyplex, or a dendrimer.

9. (canceled)

10. The compound of claim 1, wherein said polypeptide is conjugated to said transport vector through a tether molecule or wherein X is a tether molecule.

11. (canceled)

12. The compound of claim 10, wherein said tether molecule is a hydrophilic polymer.

13-14. (canceled)

15. The compound of claim 1, wherein said polypeptide is conjugated to said transport vector by a hydrophobic bond or a covalent bond.

16. The compound of claim 1, wherein said transport vector is bound to or contains a therapeutic agent.

17. The compound of claim 16, wherein said therapeutic agent is a polynucleotide, a small molecule, an anticancer agent, a polypeptide, or a hydrophobic agent.

18. The compound of claim 17, wherein said anticancer agent is paclitaxel, etoposide, doxorubicin, vinblastine, vincristine, cyclophosphamide, taxotere, melphalan, chlorambucil, or an analog thereof.

19. The compound of claim 17, wherein said polynucleotide is an RNAi agent or encodes an RNAi agent.

20. The compound of claim 19, wherein said RNAi agent is a short interfering RNA molecule (siRNA), an short hairpin RNA molecule (shRNA), a double stranded RNA molecule (dsRNA), or a microRNA molecule (miRNA).

21. The compound of claim 20, wherein said RNAi agent is capable of inhibiting expression of protein involved in cancer or a neurodegenerative disease.

22. The compound of claim 21, wherein said neurodegenerative disease is Parkinson's disease, Alzheimer's disease, dementia with Lewy bodies, or multiple system atrophy.

23. The compound of claim 20, wherein said RNAi agent inhibits or silences expression of α-synuclein, α-secretase, BACE-1, γ-secretase, amyloid precursor protein (APP), epidermal growth factor receptor (EGFR), vascular endothelial growth factor (VEGF), VEGF receptor (VEGFR), sorting nexin-6 (SNX6), LINGO-1, Nogo-A, Nogo receptor 1 (NgR-1), superoxide dismutase 1 (SOD-1), Huntingtin (Htt), or platelet-derived growth factor receptor (PDGFR).

24-27. (canceled)

28. The compound of claim 20, wherein said siRNA molecule comprises a nucleotide sequence having at least 90% sequence identity to any of the sequences set forth in SEQ ID NOS: 117-129.

29. (canceled)

30. The compound of claim 17, wherein said polynucleotide encodes a protein that is deficient in a lysosomal storage disease.

31. The compound of claim 30, wherein said polynucleotide encodes a protein selected from the group consisting of α-L-iduronidase, iduronate sulfatase, heparan N-sulfatase, α-N-acetylglucosaminidase, acetyl-CoA:α-glucosaminide acetyltransferase, N-acetylglucosamine 6-sulfatase, N-acetylgalactosamine 4-sulfatase, β-glucuronidase, sphingomyelinase, glucocerebrosidase, α-galactosidase-A, ceramidase, galactosylceramidase, arylsulfatase A, glial fibrillary acidic protein, aspartoacylase, phytanoyl-CoA hydroxylase, peroxin-7, β-galactosidase, β-hexosaminidase A, aspartylglucosaminidase (AGA), fucosidase, α-mannosidase, and sialidase.

32. The compound of claim 17, wherein said polypeptide is selected from the group consisting of a GLP-1 agonist, leptin, neurotensin, glial-derived neurotrophic factor (GDNF), and brain-derived neurotrophic factor (BDNF), or an analog thereof.

33. (canceled)

34. (canceled)

35. A composition comprising the compound of claim 1 and a pharmaceutically acceptable carrier.

36. A method of treating a subject having a neurodegenerative disease comprising administering to said subject the composition of claim 35 in a therapeutically effective amount.

37. The method of claim 36, wherein said neurodegenerative disease is multiple sclerosis, schizophrenia, epilepsy, Alzheimer's disease, Parkinson's disease, Huntington's disease, amyotrophic lateral sclerosis (ALS), or a stroke.

38. A method of treating a subject having a lysosomal storage disease comprising administering to said subject the composition of claim 35 in a therapeutically effective amount.

39. The method of claim 38, wherein said lysosomal storage disease is mucopolysaccharidosis (MPS-I; i.e., Hurler syndrome or Scheie syndrome), MPS-II (Hunter syndrome), MPS-IIIA (Sanfilippo syndrome A), MPS-IIIB (Sanfilippo syndrome B), MPS-IIIC (Sanfilippo syndrome C), MPS-IIID (Sanfilippo syndrome D), MPS-VII (Sly syndrome), Gaucher's disease, Niemann-Pick disease, Fabry disease, Farber's disease, Wolman's disease, Tay-Sachs disease, Sandhoff disease, metachromatic leukodystrophy, or Krabbé disease.

40. A method of treating a subject having a cancer comprising administering to said subject the composition of claim 35 in a therapeutically effective amount.

41. The method of claim 40, wherein said cancer is in the brain or central nervous system (CNS) and wherein said cancer is a brain tumor, a brain tumor metastasis, or a cancer that has metastasized to the brain.

42. (canceled)

43. The method of claim 40, wherein said cancer is a glioma, a glioblastoma, a hepatocellular carcinoma, or a lung cancer.

44. (canceled)

45. (canceled)

46. A method of synthesizing the compound of claim 1, comprising conjugating a polypeptide comprising an amino acid sequence having at least 70% sequence identity to SEQ ID NO:97 or to any of SEQ ID NOS:1-93, 98-105, and 107-116 to a transport vector, wherein said polypeptide is exposed on the outer surface of said transport vector, or to a component of a transport vector or to a tether molecule conjugated to said component, thereby forming a conjugate, and forming a transport vector including said conjugate.

47-52. (canceled)


SEQUENCE LISTING (148 sequences)

SEQ ID NO: 1 - protein, 19 aa, Artificial Sequence
  Synthetic Construct
TFVYGGCRAKRNNFKSAED

SEQ ID NO: 2 - protein, 19 aa, Artificial Sequence
  Synthetic Construct
TFQYGGCMGNGNNFVTEKE

SEQ ID NO: 3 - protein, 19 aa, Artificial Sequence
  Synthetic Construct
PFFYGGCGGNRNNFDTEEY

SEQ ID NO: 4 - protein, 19 aa, Artificial Sequence
  Synthetic Construct
SFYYGGCLGNKNNYLREEE

SEQ ID NO: 5 - protein, 19 aa, Artificial Sequence
  Synthetic Construct
TFFYGGCRAKRNNFKRAKY

SEQ ID NO: 6 - protein, 19 aa, Artificial Sequence
  Synthetic Construct
TFFYGGCRGKRNNFKRAKY

SEQ ID NO: 7 - protein, 19 aa, Artificial Sequence
  Synthetic Construct
TFFYGGCRAKKNNYKRAKY

SEQ ID NO: 8 - protein, 19 aa, Artificial Sequence
  Synthetic Construct
TFFYGGCRGKKNNFKRAKY

SEQ ID NO: 9 - protein, 19 aa, Artificial Sequence
  Synthetic Construct
TFQYGGCRAKRNNFKRAKY

SEQ ID NO: 10 - protein, 19 aa, Artificial Sequence
  Synthetic Construct
TFQYGGCRGKKNNFKRAKY

SEQ ID NO: 11 - protein, 19 aa, Artificial Sequence
  Synthetic Construct
TFFYGGCLGKRNNFKRAKY

SEQ ID NO: 12 - protein, 19 aa, Artificial Sequence
  Synthetic Construct
TFFYGGSLGKRNNFKRAKY

SEQ ID NO: 13 - protein, 19 aa, Artificial Sequence
  Synthetic Construct
PFFYGGCGGKKNNFKRAKY

SEQ ID NO: 14 - protein, 19 aa, Artificial Sequence
  Synthetic Construct
TFFYGGCRGKGNNYKRAKY

SEQ ID NO: 15 - protein, 19 aa, Artificial Sequence
  Synthetic Construct
PFFYGGCRGKRNNFLRAKY

SEQ ID NO: 16 - protein, 19 aa, Artificial Sequence
  Synthetic Construct
TFFYGGCRGKRNNFKREKY

SEQ ID NO: 17 - protein, 19 aa, Artificial Sequence
  Synthetic Construct
PFFYGGCRAKKNNFKRAKE

SEQ ID NO: 18 - protein, 19 aa, Artificial Sequence
  Synthetic Construct
TFFYGGCRGKRNNFKRAKD

SEQ ID NO: 19 - protein, 19 aa, Artificial Sequence
  Synthetic Construct
TFFYGGCRAKRNNFDRAKY

SEQ ID NO: 20 - protein, 19 aa, Artificial Sequence
  Synthetic Construct
TFFYGGCRGKKNNFKRAEY

SEQ ID NO: 21 - protein, 19 aa, Artificial Sequence
  Synthetic Construct
PFFYGGCGANRNNFKRAKY

SEQ ID NO: 22 - protein, 19 aa, Artificial Sequence
  Synthetic Construct
TFFYGGCGGKKNNFKTAKY

SEQ ID NO: 23 - protein, 19 aa, Artificial Sequence
  Synthetic Construct
TFFYGGCRGNRNNFLRAKY

SEQ ID NO: 24 - protein, 19 aa, Artificial Sequence
  Synthetic Construct
TFFYGGCRGNRNNFKTAKY

SEQ ID NO: 25 - protein, 19 aa, Artificial Sequence
  Synthetic Construct
TFFYGGSRGNRNNFKTAKY

SEQ ID NO: 26 - protein, 19 aa, Artificial Sequence
  Synthetic Construct
TFFYGGCLGNGNNFKRAKY

SEQ ID NO: 27 - protein, 19 aa, Artificial Sequence
  Synthetic Construct
TFFYGGCLGNRNNFLRAKY

SEQ ID NO: 28 - protein, 19 aa, Artificial Sequence
  Synthetic Construct
TFFYGGCLGNRNNFKTAKY

SEQ ID NO: 29 - protein, 19 aa, Artificial Sequence
  Synthetic Construct
TFFYGGCRGNGNNFKSAKY

SEQ ID NO: 30 - protein, 19 aa, Artificial Sequence
  Synthetic Construct
TFFYGGCRGKKNNFDREKY

SEQ ID NO: 31 - protein, 19 aa, Artificial Sequence
  Synthetic Construct
TFFYGGCRGKRNNFLREKE

SEQ ID NO: 32 - protein, 19 aa, Artificial Sequence
  Synthetic Construct
TFFYGGCRGKGNNFDRAKY

SEQ ID NO: 33 - protein, 19 aa, Artificial Sequence
  Synthetic Construct
TFFYGGSRGKGNNFDRAKY

SEQ ID NO: 34 - protein, 19 aa, Artificial Sequence
  Synthetic Construct
TFFYGGCRGNGNNFVTAKY

SEQ ID NO: 35 - protein, 19 aa, Artificial Sequence
  Synthetic Construct
PFFYGGCGGKGNNYVTAKY

SEQ ID NO: 36 - protein, 19 aa, Artificial Sequence
  Synthetic Construct
TFFYGGCLGKGNNFLTAKY

SEQ ID NO: 37 - protein, 19 aa, Artificial Sequence
  Synthetic Construct
SFFYGGCLGNKNNFLTAKY

SEQ ID NO: 38 - protein, 19 aa, Artificial Sequence
  Synthetic Construct
TFFYGGCGGNKNNFVREKY

SEQ ID NO: 39 - protein, 19 aa, Artificial Sequence
  Synthetic Construct
TFFYGGCMGNKNNFVREKY

SEQ ID NO: 40 - protein, 19 aa, Artificial Sequence
  Synthetic Construct
TFFYGGSMGNKNNFVREKY

SEQ ID NO: 41 - protein, 19 aa, Artificial Sequence
  Synthetic Construct
PFFYGGCLGNRNNYVREKY

SEQ ID NO: 42 - protein, 19 aa, Artificial Sequence
  Synthetic Construct
TFFYGGCLGNRNNFVREKY

SEQ ID NO: 43 - protein, 19 aa, Artificial Sequence
  Synthetic Construct
TFFYGGCLGNKNNYVREKY

SEQ ID NO: 44 - protein, 19 aa, Artificial Sequence
  Synthetic Construct
TFFYGGCGGNGNNFLTAKY

SEQ ID NO: 45 - protein, 19 aa, Artificial Sequence
  Synthetic Construct
TFFYGGCRGNRNNFLTAEY

SEQ ID NO: 46 - protein, 19 aa, Artificial Sequence
  Synthetic Construct
TFFYGGCRGNGNNFKSAEY

SEQ ID NO: 47 - protein, 19 aa, Artificial Sequence
  Synthetic Construct
PFFYGGCLGNKNNFKTAEY

SEQ ID NO: 48 - protein, 19 aa, Artificial Sequence
  Synthetic Construct
TFFYGGCRGNRNNFKTEEY

SEQ ID NO: 49 - protein, 19 aa, Artificial Sequence
  Synthetic Construct
TFFYGGCRGKRNNFKTEED

SEQ ID NO: 50 - protein, 19 aa, Artificial Sequence
  Synthetic Construct
PFFYGGCGGNGNNFVREKY

SEQ ID NO: 51 - protein, 19 aa, Artificial Sequence
  Synthetic Construct
SFFYGGCMGNGNNFVREKY

SEQ ID NO: 52 - protein, 19 aa, Artificial Sequence
  Synthetic Construct
PFFYGGCGGNGNNFLREKY

SEQ ID NO: 53 - protein, 19 aa, Artificial Sequence
  Synthetic Construct
TFFYGGCLGNGNNFVREKY

SEQ ID NO: 54 - protein, 19 aa, Artificial Sequence
  Synthetic Construct
SFFYGGCLGNGNNYLREKY

SEQ ID NO: 55 - protein, 19 aa, Artificial Sequence
  Synthetic Construct
TFFYGGSLGNGNNFVREKY

SEQ ID NO: 56 - protein, 19 aa, Artificial Sequence
  Synthetic Construct
TFFYGGCRGNGNNFVTAEY

SEQ ID NO: 57 - protein, 19 aa, Artificial Sequence
  Synthetic Construct
TFFYGGCLGKGNNFVSAEY

SEQ ID NO: 58 - protein, 19 aa, Artificial Sequence
  Synthetic Construct
TFFYGGCLGNRNNFDRAEY

SEQ ID NO: 59 - protein, 19 aa, Artificial Sequence
  Synthetic Construct
TFFYGGCLGNRNNFLREEY

SEQ ID NO: 60 - protein, 19 aa, Artificial Sequence
  Synthetic Construct
TFFYGGCLGNKNNYLREEY

SEQ ID NO: 61 - protein, 19 aa, Artificial Sequence
  Synthetic Construct
PFFYGGCGGNRNNYLREEY

SEQ ID NO: 62 - protein, 19 aa, Artificial Sequence
  Synthetic Construct
PFFYGGSGGNRNNYLREEY

SEQ ID NO: 63 - protein, 19 aa, Artificial Sequence
  Synthetic Construct
MRPDFCLEPPYTGPCVARI

SEQ ID NO: 64 - protein, 21 aa, Artificial Sequence
  Synthetic Construct
ARIIRYFYNAKAGLCQTFVYG

SEQ ID NO: 65 - protein, 22 aa, Artificial Sequence
  Synthetic Construct
YGGCRAKRNNYKSAEDCMRTCG

SEQ ID NO: 66 - protein, 22 aa, Artificial Sequence
  Synthetic Construct
PDFCLEPPYTGPCVARIIRYFY

SEQ ID NO: 67 - protein, 19 aa, Artificial Sequence
  Synthetic Construct
TFFYGGCRGKRNNFKTEEY

SEQ ID NO: 68 - protein, 19 aa, Artificial Sequence
  Synthetic Construct
KFFYGGCRGKRNNFKTEEY

SEQ ID NO: 69 - protein, 19 aa, Artificial Sequence
  Synthetic Construct
TFYYGGCRGKRNNYKTEEY

SEQ ID NO: 70 - protein, 19 aa, Artificial Sequence
  Synthetic Construct
TFFYGGSRGKRNNFKTEEY

SEQ ID NO: 71 - protein, 20 aa, Artificial Sequence
  Synthetic Construct
CTFFYGCCRGKRNNFKTEEY

SEQ ID NO: 72 - protein, 20 aa, Artificial Sequence
  Synthetic Construct
TFFYGGCRGKRNNFKTEEYC

SEQ ID NO: 73 - protein, 20 aa, Artificial Sequence
  Synthetic Construct
CTFFYGSCRGKRNNFKTEEY

SEQ ID NO: 74 - protein, 20 aa, Artificial Sequence
  Synthetic Construct
TFFYGGSRGKRNNFKTEEYC

SEQ ID NO: 75 - protein, 19 aa, Artificial Sequence
  Synthetic Construct
PFFYGGCRGKRNNFKTEEY

SEQ ID NO: 76 - protein, 19 aa, Artificial Sequence
  Synthetic Construct
TFFYGGCRGKRNNFKTKEY

SEQ ID NO: 77 - protein, 19 aa, Artificial Sequence
  Synthetic Construct
TFFYGGKRGKRNNFKTEEY

SEQ ID NO: 78 - protein, 19 aa, Artificial Sequence
  Synthetic Construct
TFFYGGCRGKRNNFKTKRY

SEQ ID NO: 79 - protein, 19 aa, Artificial Sequence
  Synthetic Construct
TFFYGGKRGKRNNFKTAEY

SEQ ID NO: 80 - protein, 19 aa, Artificial Sequence
  Synthetic Construct
TFFYGGKRGKRNNFKTAGY

SEQ ID NO: 81 - protein, 19 aa, Artificial Sequence
  Synthetic Construct
TFFYGGKRGKRNNFKREKY

SEQ ID NO: 82 - protein, 19 aa, Artificial Sequence
  Synthetic Construct
TFFYGGKRGKRNNFKRAKY

SEQ ID NO: 83 - protein, 19 aa, Artificial Sequence
  Synthetic Construct
TFFYGGCLGNRNNFKTEEY

SEQ ID NO: 84 - protein, 19 aa, Artificial Sequence
  Synthetic Construct
TFFYGCGRGKRNNFKTEEY

SEQ ID NO: 85 - protein, 19 aa, Artificial Sequence
  Synthetic Construct
TFFYGGRCGKRNNFKTEEY

SEQ ID NO: 86 - protein, 19 aa, Artificial Sequence
  Synthetic Construct
TFFYGGCLGNGNNFDTEEE

SEQ ID NO: 87 - protein, 19 aa, Artificial Sequence
  Synthetic Construct
TFQYGGCRGKRNNFKTEEY

SEQ ID NO: 88 - protein, 19 aa, Artificial Sequence
  Synthetic Construct
YNKEFGTFNTKGCERGYRF

SEQ ID NO: 89 - protein, 19 aa, Artificial Sequence
  Synthetic Construct
RFKYGGCLGNMNNFETLEE

SEQ ID NO: 90 - protein, 19 aa, Artificial Sequence
  Synthetic Construct
RFKYGGCLGNKNNFLRLKY

SEQ ID NO: 91 - protein, 19 aa, Artificial Sequence
  Synthetic Construct
RFKYGGCLGNKNNYLRLKY

SEQ ID NO: 92 - protein, 22 aa, Artificial Sequence
  Synthetic Construct
KTKRKRKKQRVKIAYEEIFKNY

SEQ ID NO: 93 - protein, 15 aa, Artificial Sequence
  Synthetic Construct
KTKRKRKKQRVKIAY

SEQ ID NO: 94-96 - skipped (3 entries)

SEQ ID NO: 97 - protein, 19 aa, Artificial Sequence
  Synthetic Construct
TFFYGGSRGKRNNFKTEEY

SEQ ID NO: 98 - protein, 59 aa, Artificial Sequence
  Synthetic Construct
MRPDFCLEPPYTGPCVARIIRYFYNAKAGLCQTFVYGGCRAKRNNFKSAEDCMRTCGGA

SEQ ID NO: 99 - protein, 19 aa, Artificial Sequence
  Synthetic Construct
TFFYGGCRGKRNNFKTKEY

SEQ ID NO: 100 - protein, 19 aa, Artificial Sequence
  Synthetic Construct
RFKYGGCLGNKNNYLRLKY

SEQ ID NO: 101 - protein, 19 aa, Artificial Sequence
  Synthetic Construct
TFFYGGCRAKRNNFKRAKY

SEQ ID NO: 102 - protein, 35 aa, Artificial Sequence
  Synthetic Construct
NAKAGLCQTFVYGGCLAKRNNFESAEDCMRTCGGA

SEQ ID NO: 103 - protein, 24 aa, Artificial Sequence
  Synthetic Construct
YGGCRAKRNNFKSAEDCMRTCGGA

SEQ ID NO: 104 - protein, 22 aa, Artificial Sequence
  Synthetic Construct
GLCQTFVYGGCRAKRNNFKSAE

SEQ ID NO: 105 - protein, 20 aa, Artificial Sequence
  Synthetic Construct
LCQTFVYGGCEAKRNNFKSA

SEQ ID NO: 106 - skipped

SEQ ID NO: 107 - protein, 19 aa, Artificial Sequence
  Synthetic Construct
TFFYGGSRGKRNNFKTEEY

SEQ ID NO: 108 - protein, 19 aa, Artificial Sequence
  Synthetic Construct
RFFYGGSRGKRNNFKTEEY

SEQ ID NO: 109 - protein, 19 aa, Artificial Sequence
  Synthetic Construct
RFFYGGSRGKRNNFKTEEY

SEQ ID NO: 110 - protein, 19 aa, Artificial Sequence
  Synthetic Construct
RFFYGGSRGKRNNFRTEEY

SEQ ID NO: 111 - protein, 19 aa, Artificial Sequence
  Synthetic Construct
TFFYGGSRGKRNNFRTEEY

SEQ ID NO: 112 - protein, 19 aa, Artificial Sequence
  Synthetic Construct
TFFYGGSRGRRNNFRTEEY

SEQ ID NO: 113 - protein, 20 aa, Artificial Sequence
  Synthetic Construct
CTFFYGGSRGKRNNFKTEEY

SEQ ID NO: 114 - protein, 20 aa, Artificial Sequence
  Synthetic Construct
TFFYGGSRGKRNNFKTEEYC

SEQ ID NO: 115 - protein, 20 aa, Artificial Sequence
  Synthetic Construct
CTFFYGGSRGRRNNFRTEEY

SEQ ID NO: 116 - protein, 20 aa, Artificial Sequence
  Synthetic Construct
TFFYGGSRGRRNNFRTEEYC

SEQ ID NO: 117 - RNA, 19 nt, Artificial Sequence
  Synthetic Construct
ggagcugcccaugagaaau

SEQ ID NO: 118 - RNA, 19 nt, Artificial Sequence
  Synthetic Construct
auuucucaugggcagcucc

SEQ ID NO: 119 - DNA, 19 bp, Artificial Sequence
  Synthetic Construct
ggagtaccctgatgagatc

SEQ ID NO: 120 - DNA, 21 bp, Artificial Sequence
  Synthetic Construct
aaggaccagttgggcaagaat

SEQ ID NO: 121 - DNA, 21 bp, Artificial Sequence
  Synthetic Construct
aacagtggctgagaagaccaa

SEQ ID NO: 122 - DNA, 21 bp, Artificial Sequence
  Synthetic Construct
aaaaaggaccagttgggcaag

SEQ ID NO: 123 - DNA, 21 bp, Artificial Sequence
  Synthetic Construct
aaaaggaccagttgggcaaga

SEQ ID NO: 124 - DNA, 21 bp, Artificial Sequence
  Synthetic Construct
aaaggaccagttgggcaagaa

SEQ ID NO: 125 - DNA, 21 bp, Artificial Sequence
  Synthetic Construct
aagatatgcctgtggatcctg

SEQ ID NO: 126 - DNA, 21 bp, Artificial Sequence
  Synthetic Construct
aaatgccttctgaggaagggt

SEQ ID NO: 127 - DNA, 21 bp, Artificial Sequence
  Synthetic Construct
aatgccttctgaggaagggta

SEQ ID NO: 128 - DNA, 21 bp, Artificial Sequence
  Synthetic Construct
aagactacgaacctgaagcct

SEQ ID NO: 129 - DNA, 21 bp, Artificial Sequence
  Synthetic Construct
aagactgtggctacaacattc

SEQ ID NO: 130 - protein, 39 aa, Artificial Sequence
  Synthetic Construct
  MOD_RES (32)..(32): Xaa is Cys or Ser
  MOD_RES (39)..(39): Xaa is Lys or Ser
  MISC_FEATURE (39)..(39): C terminal is amidated
HGEGTFTSDLSKQMEEEAVRLFIEWLKNGGPXSGAPPPX

SEQ ID NO: 131 - protein, 39 aa, Artificial Sequence
  Synthetic Construct
  MISC_FEATURE (1)..(39): C-terminal may or may not be amidated
  MOD_RES (1)..(1): Xaa is His, Arg, or Tyr
  MOD_RES (2)..(2): Xaa is Ser, Gly, Ala, or Thr
  MOD_RES (3)..(3): Xaa is Asp or Glu
  MOD_RES (6)..(6): Xaa is Phe, Tyr or Nal
  MOD_RES (7)..(7): Xaa is Thr or Ser
  MOD_RES (8)..(8): Xaa is Ser or Thr
  MOD_RES (9)..(9): Xaa is Asp or Glu
  MOD_RES (10)..(10): Xaa is Leu, Ile, Val, pGly or Met
  MOD_RES (14)..(14): Xaa is Leu, Ile, pGly, Val or Met
  MOD_RES (22)..(22): Xaa is Phe, Tyr or Nal
  MOD_RES (23)..(23): Xaa is Ile, Val, Leu, pGly, t-BuGly or Met
  MOD_RES (24)..(24): Xaa is Glu or Asp
  MOD_RES (25)..(25): Xaa is Trp, Phe, Tyr or Nal
  MOD_RES (31)..(31): Xaa is Pro, HPro, 3Hyp, 4Hyp, TPro, N-alkylglycine, N-alkyl-pGly or N-alkylalanine
  MOD_RES (36)..(38): Xaa is Pro, HPro, 3Hyp, 4Hyp, TPro, N-alkylglycine, N-alkyl-pGly or N-alkylalanine
  MOD_RES (39)..(39): Xaa is Ser, Thr or Tyr
XXXGTXXXXXSKQXEEEAVRLXXXXLKNGGXSSGAXXXX

SEQ ID NO: 132 - protein, 38 aa, Artificial Sequence
  Synthetic Construct
  MISC_FEATURE (1)..(38): C-terminal may or may not be amidated
  MOD_RES (1)..(1): Xaa is His, Arg, or Tyr
  MOD_RES (2)..(2): Xaa is Ser, Gly, Ala, or Thr
  MOD_RES (3)..(3): Xaa is Asp or Glu
  MOD_RES (5)..(5): Xaa is Ala or Thr
  MOD_RES (6)..(6): Xaa is Ala, Phe, Tyr or Nal
  MOD_RES (7)..(7): Xaa is Thr or Ser
  MOD_RES (8)..(8): Xaa is Ala, Ser, or Thr
  MOD_RES (9)..(9): Xaa is Asp or Glu
  MOD_RES (10)..(10): Xaa is Ala, Leu, Ile, Val, pGly, or Met
  MOD_RES (11)..(11): Xaa is Ala or Ser
  MOD_RES (12)..(12): Xaa is Ala or Lys
  MOD_RES (13)..(13): Xaa is Ala or Gln
  MOD_RES (14)..(14): Xaa is Ala, Leu, Ile, pGly, Val, or Met
  MOD_RES (15)..(17): Xaa is Ala or Glu
  MOD_RES (19)..(19): Xaa is Ala or Val
  MOD_RES (20)..(20): Xaa is Ala or Arg
  MOD_RES (21)..(21): Xaa is Ala or Leu
  MOD_RES (22)..(22): Xaa is Phe, Tyr, or Nal
  MOD_RES (23)..(23): Xaa is Ile, Val, Leu, pGly, t-BuGly, or Met
  MOD_RES (24)..(24): Xaa is Ala, Glu, or Asp
  MOD_RES (25)..(25): Xaa is Ala, Trp, Phe, Tyr, or Nal
  MOD_RES (26)..(26): Xaa is Ala or Leu
  MOD_RES (27)..(27): Xaa is Ala or Lys
  MISC_FEATURE (28)..(38): C-terminal may or may not be amidated
  MOD_RES (28)..(28): Xaa is Ala or Asn
  MOD_RES (29)..(29): Xaa is Gly or is absent
  MOD_RES (30)..(30): Xaa is Gly or is absent
  MOD_RES (31)..(31): Xaa is Pro, HPro, 3Hyp, 4Hyp, TPro, N-alkylglycine, N-alkyl-pGly, or N-alkylalanine or is absent
  MOD_RES (32)..(32): Xaa is Ser or is absent
  MOD_RES (33)..(33): Xaa is Ser or is absent
  MOD_RES (34)..(34): Xaa is Gly or is absent
  MOD_RES (35)..(35): Xaa is Ala or is absent
  MOD_RES (36)..(36): Xaa is Pro, HPro, 3Hyp, 4Hyp, TPro, N-alkylglycine, N-alkyl-pGly, or N-alkylalanine or is absent
  MOD_RES (37)..(37): Xaa is Pro, HPro, 3Hyp, 4Hyp, TPro, N-alkylglycine, N-alkyl-pGly, or N-alkylalanine or is absent
  MOD_RES (38)..(38): Xaa is Pro, HPro, 3Hyp, 4Hyp, TPro, N-alkylglycine, N-alkyl-pGly, or N-alkylalanine or is absent
XXXGXXXXXXXXXXXXXAXXXXXXXXXXXXXXXXXXXX

SEQ ID NO: 133 - protein, 39 aa, Artificial Sequence
  Synthetic Construct
  MISC_FEATURE (1)..(39): C-terminal may or may not be amidated
  MOD_RES (1)..(1): Xaa is His, Arg, Tyr, Ala, Norval, Val, or Norleu
  MOD_RES (2)..(2): Xaa is Ser, Gly, Ala, or Thr
  MOD_RES (3)..(3): Xaa is Ala, Asp, or Glu
  MOD_RES (4)..(4): Xaa is is Ala, Norval, Val, Norleu, or Gly
  MOD_RES (5)..(5): Xaa is Ala or Thr
  MOD_RES (6)..(6): Xaa is Phe, Tyr, or Nal
  MOD_RES (7)..(7): Xaa is Thr or Ser
  MOD_RES (8)..(8): Xaa is Ala, Ser or Thr
  MOD_RES (9)..(9): Xaa is Ala, Norval, Val, Norleu, Asp, or Glu
  MOD_RES (10)..(10): Xaa is Ala, Leu, Ile, Val, pGly, or Met
  MOD_RES (11)..(11): Xaa is Ala or Ser
  MOD_RES (12)..(12): Xaa is Ala or Lys
  MOD_RES (13)..(13): Xaa is Ala or Gln
  MOD_RES (14)..(14): Xaa is Ala, Leu, Ile, pGly, Val, or Met
  MOD_RES (15)..(15): Xaa is Ala or Glu
  MOD_RES (16)..(16): Xaa is Ala or Glu
  MOD_RES (17)..(17): Xaa is Ala or Glu
  MOD_RES (19)..(19): Xaa is Ala or Val
  MOD_RES (20)..(20): Xaa is Ala or Arg
  MOD_RES (21)..(21): Xaa is Ala or Leu
  MOD_RES (22)..(22): Xaa is Phe, Tyr, or Nal
  MOD_RES (23)..(23): Xaa is Ile, Val, Leu, pGly, t-BuG, or Met
  MOD_RES (24)..(24): Xaa is Ala, Glu, or Asp
  MOD_RES (25)..(25): Xaa is Ala, Trp, Phe, Tyr, or Nal
  MOD_RES (26)..(26): Xaa is Ala or Leu
  MOD_RES (27)..(27): Xaa is Ala or Lys
  MISC_FEATURE (28)..(39): C-terminal may or may not be amidated
  MOD_RES (28)..(28): Xaa is Ala or Asn
  MOD_RES (29)..(29): Xaa is Gly or is absent
  MOD_RES (30)..(30): Xaa is Gly or is absent
  MOD_RES (31)..(31): Xaa is Pro, HPro, 3Hyp, 4Hyp, TPro, N-alkylglycine, N-alkyl-pGly, or N-alkylalanine or is absent
  MOD_RES (32)..(32): Xaa is Ser or is absent
  MOD_RES (33)..(33): Xaa is Ser or is absent
  MOD_RES (34)..(34): Xaa is Gly or is absent
  MOD_RES (35)..(35): Xaa is Ala or is absent
  MOD_RES (36)..(36): Xaa is Pro, HPro, 3Hyp, 4Hyp, TPro, N-alkylglycine, N-alkyl-pGly, or N-alkylalanine or is absent
  MOD_RES (37)..(37): Xaa is Pro, HPro, 3Hyp, 4Hyp, TPro, N-alkylglycine, N-alkyl-pGly, or N-alkylalanine or is absent
  MOD_RES (38)..(38): Xaa is Pro, HPro, 3Hyp, 4Hyp, TPro, N-alkylglycine, N-alkyl-pGly, or N-alkylalanine or is absent
  MOD_RES (39)..(39): Xaa is Pro, HPro, 3Hyp, 4Hyp, TPro, N-alkylglycine, N-alkyl-pGly, or N-alkylalanine or is absent
XXXXXXXXXXXXXXXXXAXXXXXXXXXXXXXXXXXXXXX

SEQ ID NO: 134 - protein, 40 aa, Artificial Sequence
  Synthetic Construct
  MISC_FEATURE (1)..(40): C-terminal may or may not be amidated
  MOD_RES (14)..(14): Xaa is Arg, Leu, Ile, or Met
  MOD_RES (20)..(20): Xaa is His, Arg, or Lys
  MOD_RES (40)..(40): Xaa is Arg or Lys
HGEGTFTSDLSKQXEEEAVXLFIEWLKNGGPSSGAPPPSX

SEQ ID NO: 135 - protein, 32 aa, Artificial Sequence
  Synthetic Construct
  MOD_RES (6)..(6): Xaa is Phe or Tyr
  MOD_RES (14)..(14): Xaa is Met, Ile, or Leu
  MOD_RES (20)..(20): Xaa is Lys
  MOD_RES (30)..(30): Xaa is Gly or is absent
  MOD_RES (32)..(32): Xaa is Arg or is absent
HGEGTXTSDLSKQXEEEAVXLFIEWLKNGXPX

SEQ ID NO: 136 - protein, 31 aa, Artificial Sequence
  Synthetic Construct
  MOD_RES (29)..(29): Xaa is Gly or is absent
  MOD_RES (30)..(30): Xaa is Arg or is absent
  MOD_RES (31)..(31): Xaa is Gly or is absent
HAEGTFTSDVSSYLEGQAAKEFIAWLVKXXX

SEQ ID NO: 137 - protein, 30 aa, Artificial Sequence
  Synthetic Construct
  MISC_FEATURE (1)..(30): C-terminal may or may not be amidated
  MOD_RES (1)..(1): Xaa is Ala or is absent
  MOD_RES (2)..(2): Xaa is Glu or is absent
  MOD_RES (3)..(3): Xaa is Gly or is absent
  MOD_RES (4)..(4): Xaa is Thr or is absent
  MOD_RES (5)..(5): Xaa is Phe or is absent
  MOD_RES (6)..(6): Xaa is Thr or is absent
  MOD_RES (7)..(7): Xaa is Ser or absent
  MOD_RES (8)..(8): Xaa is Asp or is absent
  MOD_RES (9)..(9): Xaa is Val or is absent
  MOD_RES (10)..(10): Xaa is Ser or is absent
  MOD_RES (27)..(27): Xaa is Lys or Arg
  MOD_RES (30)..(30): Xaa is Gly or is absent
XXXXXXXXXXSYLEGQAAKEFIAWLVXGRX

SEQ ID NO: 138 - protein, 30 aa, Artificial Sequence
  Synthetic Construct
  MOD_RES (28)..(28): Xaa is Lys
  MOD_RES (29)..(29): Xaa is Gly or is absent
  MOD_RES (30)..(30): Xaa is Arg or is absent
HAEGTFTSDVSSYLEGQAAKEFIAWLVXXX

SEQ ID NO: 139 - protein, 30 aa, Artificial Sequence
  Synthetic Construct
  MOD_RES (30)..(30): -CO-R1, where R1 is OH, OM, or -NR2R3; M is a pharmaceutically acceptable cation or a lower branched or unbranched alkyl group; R2 and R3 are independently selected from the group consisting of hydrogen and a lower branched or unbranched alkyl group
HAEGTFTSDVSSYLEGQAAKEFIAWLVKGR

SEQ ID NO: 140 - protein, 31 aa, Artificial Sequence
  Synthetic Construct
  MISC_FEATURE (1)..(31): C-terminal may or may not be amidated
  MOD_RES (1)..(1): Xaa is His, D-His, desamino-His, 2-amino-His, beta-hydroxy-His, homohistidine, alpha-fluoromethyl-His, or alpha-methyl-His
  MOD_RES (2)..(2): Xaa is Met, Asp, Lys, Thr, Leu, Asn, Gln, Phe, Val, or Tyr
  MOD_RES (15)..(15): Xaa is Glu, Gln, Ala, Thr, Ser, or Gly
  MOD_RES (21)..(21): Xaa is Glu, Gln, Ala, Thr, Ser, or Gly
  MOD_RES (31)..(31): Xaa is Gly or is absent
XXEGTFTSDVSSYLXGQAAKXFIAWLVKGRX

SEQ ID NO: 141 - protein, 31 aa, Artificial Sequence
  Synthetic Construct
  MISC_FEATURE (1)..(31): C-terminal may or may not be amidated
  MOD_RES (1)..(1): Xaa is 4-imidazopropionyl (des-amino-histidyl), 4-imidazoacetyl or 4-imidazo-alpha,alpha-dimethyl-acetyl
  MOD_RES (20)..(20): Xaa is Lys or Arg
  MOD_RES (28)..(28): Xaa is Lys or Lys having a C6-10 unbranched acyl bound to its side chain
  MOD_RES (31)..(31): Xaa is Gly or is absent
XAEGTFTSDVSSYLEGQAAXEFIAWLVXGRX

SEQ ID NO: 142 - protein, 32 aa, Artificial Sequence
  Synthetic Construct
  MISC_FEATURE (1)..(31): C-terminal may or may not be amidated
  MOD_RES (2)..(2): Xaa is Gly, Ala, Val, Leu, Ile, Ser, or Thr
  MOD_RES (5)..(5): Xaa is Asp, Glu, Arg, Thr, Ala, Lys, or His
  MOD_RES (6)..(6): Xaa is His, Trp, Phe, or Tyr
  MOD_RES (10)..(10): Xaa is Leu, Ser, Thr, Trp, His, Phe, Asp, Val, Tyr, Glu, or Ala
  MOD_RES (16)..(16): Xaa is Gly, Asp, Glu, Gln, Asn, Lys, Arg, Cys, or Cya
  MOD_RES (17)..(17): Xaa is His, Asp, Lys, Glu, or Gln
  MOD_RES (18)..(18): Xaa is Glu, His, Ala, or Lys
  MOD_RES (20)..(20): Xaa is Asp, Lys, Glu, or His
  MOD_RES (21)..(21): Xaa is Ala, Glu, His, Phe, Tyr, Trp, Arg, or Lys
  MOD_RES (25)..(25): Xaa is Ala, Glu, Asp, Ser, or His
  MOD_RES (27)..(27): Xaa is Asp, Arg, Val, Lys, Ala, Gly, or Glu
  MOD_RES (28)..(28): Xaa is Glu, Lys, or Asp
  MOD_RES (29)..(29): Xaa is Thr, Ser, Lys, Arg, Trp, Tyr, Phe, Asp, Gly, Pro, His, or Glu
  MOD_RES (30)..(30): Xaa is Arg, Glu, or His
  MOD_RES (31)..(31): Xaa is Lys, Arg, Thr, Ser, Glu, Asp, Trp, Tyr, Phe, His, Gly, or is absent
  MOD_RES (32)..(32): Xaa is Pro or is absent
HXEGXXTSDXSSYLEXXXAXXFIAXLXXXXXX

SEQ ID NO: 143 - protein, 32 aa, Artificial Sequence
  Synthetic Construct
  MISC_FEATURE (1)..(31): C-terminal may or may not be amidated
  MOD_RES (2)..(2): Xaa is Gly, Ala, Val, Leu, Ile, Ser, or Thr
  MOD_RES (6)..(6): Xaa is His, Trp, Phe, or Tyr
  MOD_RES (10)..(10): Xaa is Leu, Ser, Thr, Trp, His, Phe, Asp, Val, Glu, or Ala
  MOD_RES (16)..(16): Xaa is Gly, Asp, Glu, Gln, Asn, Lys, Arg, Cys, or Cya
  MOD_RES (17)..(17): Xaa is His, Asp, Lys, Glu, or Gln
  MOD_RES (20)..(20): Xaa is Asp, Lys, Glu, or His
  MOD_RES (24)..(24): Xaa is Ala, Glu, Asp, Ser, or His
  MOD_RES (29)..(29): Xaa is Thr, Ser, Lys, Arg, Trp, Tyr, Phe, Asp, Gly, Pro, His, or Glu
  MOD_RES (31)..(31): Xaa is Lys, Arg, Thr, Ser, Glu, Asp, Trp, Tyr, Phe, His, Gly, or is absent
  MOD_RES (32)..(32): Xaa is Pro or is absent
HXEGTXTSDXSSYLEXXAAXEFIXWLVKXRXX

SEQ ID NO: 144 - protein, 32 aa, Artificial Sequence
  Synthetic Construct
  MISC_FEATURE (1)..(31): C-terminal may or may not be amidated
  MOD_RES (2)..(2): Xaa is Gly, Ala, Val, Leu, Ile, Ser, or Thr
  MOD_RES (16)..(16): Xaa is Gly, Asp, Glu, Gln, Asn, Lys, Arg, Cys, or Cya
  MOD_RES (17)..(17): Xaa is His, Asp, Lys, Glu, or Gln
  MOD_RES (21)..(21): Xaa is Ala, Glu, His, Phe, Tyr, Trp, Arg, or Lys
  MOD_RES (24)..(24): Xaa is Ala, Glu, Asp, Ser, or His
  MOD_RES (31)..(31): Xaa is Lys, Arg, Thr, Ser, Glu, Asp, Trp, Tyr, Phe, His, -NH2, Gly, or is absent
  MOD_RES (32)..(32): Xaa is Pro or is absent
HXEGTFTSDVSSYLEXXAAKXFIXWLVKGRXX

SEQ ID NO: 145 - protein, 31 aa, Artificial Sequence
  Synthetic Construct
  MISC_FEATURE (1)..(31): C-terminal may or may not be amidated
  MOD_RES (1)..(1): Xaa is L-His, D-His, desamino-His, 2amino-His, beta-hydroxy-His, homo-His, alpha-fluoromethyl-His or alpha- methyl-His
  MOD_RES (2)..(2): Xaa is Gly, Ala, Val, Leu, Ile, Ser or Thr
  MOD_RES (16)..(16): Xaa is Asp, Glu, Gln, Asn, Lys, Arg, Cys, or Cya
  MOD_RES (31)..(31): Xaa is Gly or is absent
XXEGTFTSDVSSYLEXQAAKEFIAWLVKGRX

SEQ ID NO: 146 - protein, 31 aa, Artificial Sequence
  Synthetic Construct
  MISC_FEATURE (1)..(31): C-terminal may or may not be amidated
  MOD_RES (1)..(1): Xaa is L-His, D-His, desamino-His, 2-amino-His, beta-hydroxy-His, homohistidine, alpha-fluoromethyl-His, or alpha-methyl-His
  MOD_RES (2)..(2): Xaa is Ala, Gly, Val, Leu, Ile, Ser, or Thr
  MOD_RES (6)..(6): Xaa is Phe, Trp, or Tyr
  MOD_RES (10)..(10): Xaa is Val, Trp, Ile, Leu, Phe, or Tyr
  MOD_RES (12)..(12): Xaa is Ser, Trp, Tyr, Phe, Lys, Ile, Leu, or Val
  MOD_RES (13)..(13): Xaa is Tyr, Trp, or Phe
  MOD_RES (14)..(14): Xaa is Leu, Phe, Tyr, or Trp
  MOD_RES (16)..(16): Xaa is Gly, Glu, Asp, or Lys
  MOD_RES (19)..(19): Xaa is Ala, Val, Ile, or Leu
  MOD_RES (21)..(21): Xaa is Glu, Ile, or Ala
  MOD_RES (24)..(24): Xaa is Ala or Glu
  MOD_RES (27)..(27): Xaa is Val or Ile
  MOD_RES (31)..(31): Xaa is Gly, His, or is absent
XXEGTXTSDXSXXXEXQAXKXFIXWLXKGRX

SEQ ID NO: 147 - protein, 31 aa, Artificial Sequence
  Synthetic Construct
  MISC_FEATURE (1)..(31): C-terminal may or may not be amidated
  MOD_RES (1)..(1): Xaa is L-His, D-His, desamino-His, 2-amino-His, beta-hydroxy-His, homohistidine, alpha-fluoromethyl-His, or alpha-methyl-His
  MOD_RES (2)..(2): Xaa is Gly, Ala, Val, Leu, Ile, Ser, or Thr
  MOD_RES (10)..(10): Xaa is Val, Phe, Tyr, or Trp
  MOD_RES (12)..(12): Xaa is Ser, Tyr, Trp, Phe, Lys, Ile, Leu, or Val
  MOD_RES (16)..(16): Xaa is Gly, Glu, Asp, or Lys
  MOD_RES (19)..(19): Xaa is Ala, Val, Ile, or Leu
  MOD_RES (27)..(27): Xaa is Val or Ile
  MOD_RES (31)..(31): Xaa is Gly or is absent
XXEGTFTSDXSXYLEXQAXKEFIAWLXKGRX

SEQ ID NO: 148 - protein, 13 aa, Artificial Sequence
  Synthetic Construct
  MOD_RES (1)..(1): Xaa is optionally present. When present Xaa is an amino acid or polypeptide containing from about 1-15 amino acid residues, an R group, an R-C(O) (amide) group, a carbamate group RO-C(O), a urea R4R5N-C(O), a sulfonamido R-SO2, or R4R5N-SO2. See specification.
  MOD_RES (2)..(10): Xaa is a naturally or nonnaturally occurring amino acid residue
  MOD_RES (11)..(11): Xaa is an amino acid residue.  See specification as filed for detailed description of substitutions and preferred embodiments.
  MOD_RES (12)..(12): Xaa is an amino acid residue.  See specification as filed for detailed description of substitutions and preferred embodiments.
  MOD_RES (13)..(13): Xaa is optionally present. When present Xaa is an amino acid or polypeptide containing from about 1-15 amino acid residues, an R group, an R-C(O) (amide) group, a carbamate group RO-C(O), a urea R4R5N-C(O), a sulfonamido R-SO2, or R4R5N-SO2. See specification.
XXXXXXXXXXXXX